(19) United States
(12) Patent Application Publication
(10) Pub. No.: US 2023/0192841 A1
(43) Pub. Date: Jun. 22, 2023

(54) CLAUDIN18.2 BINDING MOIETIES AND USES THEREOF

(71) Applicant: NANJING LEGEND BIOTECH CO., LTD., Nanjing, Jiangsu (CN)

(72) Inventors: Xiaohu FAN, Edmonton (CA); Qiuchuan ZHUANG, Nanjing, Jiangsu (CN); Xu FANG, Nanjing, Jiangsu (CN); Qingshan ZHANG, Nanjing, Jiangsu (CN); Manman XU, Nanjing, Jiangsu (CN); Meng FENG, Nanjing, Jiangsu (CN)

(73) Assignee: NANJING LEGEND BIOTECH CO., LTD., Nanjing, Jiangsu (CN)

(21) Appl. No.: 17/786,819

(22) PCT Filed: Dec. 24, 2020
(86) PCT No.: PCT/CN2020/139143

§ 371 Date: Jun. 17, 2022

(30) Foreign Application Priority Data
Dec. 27, 2019 (WO) PCT/CN2019/129095

(51) Int. Cl. C07K 16/28; C07K 14/725; C07K 14/705; A61K 35/17; A61P 35/00; A61P 1/00

(52) U.S. Cl. CPC C07K 16/28; A61K 35/17; A61P 1/00; A61P 35/00; C07K 14/7051; C07K 14/70517; C07K 14/70578; C07K 2317/22; C07K 2317/24; C07K 2317/73; C07K 2317/92; C07K 2317/569; C07K 2319/02; C07K 2319/03

(57) ABSTRACT

Single domain antibodies that bind to Claudin18.2, and chimeric antigen receptors comprising same are provided. Further provided are engineered immune effector cells (such as T cells) comprising the chimeric antigen receptors. Pharmaceutical compositions, kits and methods of treating a disease or disorder are also provided.

### CROSS REFERENCE

This application claims benefit of priority of International Patent Application No. PCT/CN2019/129095 filed on Dec. 27, 2019, the content of which is incorporated herein by reference in its entirety.

### SEQUENCE LISTING

This application incorporates by reference a Sequence Listing submitted with this application as a text format, entitled “14651-011-228_SEQ_LISTING.txt,” created on Dec. 24, 2020 having a size of 124,111 bytes.

### 1. FIELD

The present disclosure relates to anti-Claudin18.2 single domain antibodies, chimeric antigen receptors, engineered immune effector cells, and methods of use thereof. The present disclosure further relates to activation and expansion of cells for therapeutic uses, especially to chimeric antigen receptor-based T cell immunotherapies.

### 2. BACKGROUND

Gastric cancer (GC) is one of the leading causes of cancer-related deaths worldwide, with a 5-year survival rate of 5%-20% (Ferlay J. et al., International journal of cancer 136(5):E359-386 (2015)). Since the early disease symptoms are mostly nonspecific, in countries where routine screening is inaccessible, most patients with GC or cancers of the gastro-esophageal junction GEJ) cancers are diagnosed at an advanced stage (Maconi G. et al., (2008) World J Gastroenterol 14(8):1149-1155). Chemotherapy of platinum and fluoropyrimidine derivatives is currently recommended as the first-line therapy for unresectable or metastatic GC/GEJ cancers. However, disease progression was eventually observed in patients receiving such treatment, with a median progression-free survival of 5-7 months and a median overall survival of 9-11 months (Roberto I. et al., PLoS One 9(9):e108940 (2014); Kim H. S. et al., Annals of Oncology 24(11):2850-2854 (2013)).

Claudin18.2 is a splice variant of Claudin 18, a member of the claudin family of tetrameric membrane proteins that are expressed at the junction of epithelial cells and establish a paracellular barrier and control the flow of molecules between cells, playing critical roles in cell signaling and epithelial cell polarity maintenance (Singh et al., J Oncol. 2010: 541957 (2010)). The expression of Claudin18.2 is strictly restricted to the tight junction of the gastric mucosa in normal tissues and is buried in the supramolecular complex, and therefore Claudin18.2 in normal tissues is largely inaccessible to intravenous (IV) antibodies. However, Claudin18.2 becomes exposed on cancer cell surfaces, and its expression is found in up to 80% of GC tumors, and it is also abnormally activated in many other types of tumors, such as pancreatic cancer, esophageal cancer, ovarian cancer, and lung cancers such as non-small cell lung cancer (NSCLC), colon cancer, hepatic cancer, head-neck cancer, gallbladder cancers and metastases thereof (Sahin U. et al., Clinical Cancer Research 14(23):7624-7634 (2018)). Although anti-Claudin18.2 antibodies and chimeric antigen receptors (CARs) have been studied for years, there is still a need for improved Claudin18.2-binding therapeutic molecules and engineered Claudin18.2-targeting cells. For example, there is a need to develop stable and small-sized Claudin18.2 binding molecules for use in more effective or efficient CAR-T cell therapies.

### 3. SUMMARY

The present application provides a binding moiety that specifically binds to Claudin18.2 comprising one or more anti-Claudin18.2 single domain antibodies (sdAbs) or antigen binding fragments thereof, chimeric antigen receptors (CARs) comprising one or more anti-Claudin18.2 sdAbs or antigen binding fragments thereof (such as VHH fragments), engineered immune effector cells, and methods of use thereof, e.g., in cancer immunotherapy.

In one aspect, the present disclosure provides a binding moiety that specifically binds to Claudin18.2, comprising a single domain antibody or an antigen binding fragment thereof comprising (i) a CDR1 region comprising an amino acid sequence selected from the group consisting of SEQ ID NOs:1-11 and 113-125; (ii) a CDR2 region comprising an amino acid sequence selected from the group consisting of SEQ ID NOs: 12-23; and (iii) a CDR3 region comprising an amino acid sequence selected from the group consisting of SEQ ID NOs:24-37 and 126-139, or a variant thereof comprising up to 5 amino acid substitutions, deletions and/or insertions (e.g., one, two, three, four or five amino acid substitutions, deletions and/or insertions) in each of CDR1, CDR2, and CDR3.

In some embodiments, the Claudin18.2 binding moiety comprises a single domain antibody or an antigen binding fragment thereof comprising any one of the following: (1) a CDR1 comprising the amino acid sequence of SEQ ID NO:1 or SEQ ID NO:113; a CDR2 comprising the amino acid sequence of SEQ ID NO:12; and a CDR3 comprising the amino acid sequence of SEQ ID NO:24 or SEQ ID NO:126; (2) a CDR1 comprising the amino acid sequence of SEQ ID NO:2 or SEQ ID NO:114; a CDR2 comprising the amino acid sequence of SEQ ID NO:13; and a CDR3 comprising the amino acid sequence of SEQ ID NO:25 or SEQ ID NO:127; (3) a CDR1 comprising the amino acid sequence of SEQ ID NO:3 or SEQ ID NO:115; a CDR2 comprising the amino acid sequence of SEQ ID NO:14; and a CDR3 comprising the amino acid sequence of SEQ ID NO:26 or SEQ ID NO:128; (4) a CDR1 comprising the amino acid sequence of SEQ ID NO:4 or SEQ ID NO:116; a CDR2 comprising the amino acid sequence of SEQ ID NO:15; and a CDR3 comprising the amino acid sequence of SEQ ID NO:27 or SEQ ID NO:129; (5) a CDR1 comprising the amino acid sequence of SEQ ID NO:5 or SEQ ID NO:117; a CDR2 comprising the amino acid sequence of SEQ ID NO:16; and a CDR3 comprising the amino acid sequence of SEQ ID NO:28 or SEQ ID NO:130; (6) a CDR1 comprising the amino acid sequence of SEQ ID NO:6 or SEQ ID NO:118; a CDR2 comprising the amino acid sequence of SEQ ID NO:17; and a CDR3 comprising the amino acid sequence of SEQ ID NO:29 or SEQ ID NO:131; (7) a CDR1 comprising the amino acid sequence of SEQ ID NO:7 or SEQ ID NO:119; a CDR2 comprising the amino acid sequence of SEQ ID NO:18; and a CDR3 comprising the amino acid sequence of SEQ ID NO:30 or SEQ ID NO:132; (8) a CDR1 comprising the amino acid sequence of SEQ ID NO:8 or SEQ ID NO:120; a CDR2 comprising the amino acid sequence of SEQ ID NO:19; and a CDR3 comprising the amino acid sequence of SEQ ID NO:31 or SEQ ID NO:133; (9) a CDR1 comprising the amino acid sequence of SEQ ID NO:1 or SEQ ID NO:121; a CDR2 comprising the amino acid sequence of SEQ ID NO:20; and a CDR3 comprising the amino acid sequence of SEQ ID NO:32 or SEQ ID NO:134; (10) a CDR1 comprising the amino acid sequence of SEQ ID NO:5 or SEQ ID NO:122; a CDR2 comprising the amino acid sequence of SEQ ID NO:21; and a CDR3 comprising the amino acid sequence of SEQ ID NO:33 or SEQ ID NO:135; (11) a CDR1 comprising the amino acid sequence of SEQ ID NO:9 or SEQ ID NO:123; a CDR2 comprising the amino acid sequence of SEQ ID NO:22; and a CDR3 comprising the amino acid sequence of SEQ ID NO:34 or SEQ ID NO:136; (12) a CDR1 comprising the amino acid sequence of SEQ ID NO:10 or SEQ ID NO:124; a CDR2 comprising the amino acid sequence of SEQ ID NO:23; and a CDR3 comprising the amino acid sequence of SEQ ID NO:35 or SEQ ID NO:137; (13) a CDR1 comprising the amino acid sequence of SEQ ID NO:5 or SEQ ID NO:122; a CDR2 comprising the amino acid sequence of SEQ ID NO:21; and a CDR3 comprising the amino acid sequence of SEQ ID NO:36 or SEQ ID NO:138; (14) a CDR1 comprising the amino acid sequence of SEQ ID NO:11 or SEQ ID NO:125; a CDR2 comprising the amino acid sequence of SEQ ID NO:13; and a CDR3 comprising the amino acid sequence of SEQ ID NO:37 or SEQ ID NO:139. In some embodiments, provided herein are variants of these Claudin18.2 binding moieties comprising up to about 5 amino acid substitutions, deletions and/or insertions (e.g., one, two, three, four or five amino acid substitutions, deletions and/or insertions) in the CDRs.

In some embodiments, the Claudin18.2 binding moiety provided herein comprises a CDR1, a CDR2, and a CDR3 from a binding moiety comprising a single domain antibody or an antigen binding fragment thereof having an amino acid sequence selected from the group consisting of SEQ ID NOs: 38-51 and 77-85. In some embodiments, provided herein is a binding moiety that specifically binds to Claudin18.2, comprising a single domain antibody or an antigen binding fragment thereof comprising (i) a CDR1, a CDR2, and a CDR3 having the amino acid sequences of the CDR1, CDR2, and CDR3, respectively, as set forth in SEQ ID NO:38; (ii) a CDR1, a CDR2, and a CDR3 having the amino acid sequences of the CDR1, CDR2, and CDR3, respectively, as set forth in SEQ ID NO:39; (iii) a CDR1, a CDR2, and a CDR3 having the amino acid sequences of the CDR1, CDR2, and CDR3, respectively, as set forth in SEQ ID NO:40; (iv) a CDR1, a CDR2, and a CDR3 having the amino acid sequences of the CDR1, CDR2, and CDR3, respectively, as set forth in SEQ ID NO:41; (v) a CDR1, a CDR2, and a CDR3 having the amino acid sequences of the CDR1, CDR2, and CDR3, respectively, as set forth in SEQ ID NO:42; (vi) a CDR1, a CDR2, and a CDR3 having the amino acid sequences of the CDR1, CDR2, and CDR3, respectively, as set forth in SEQ ID NO:43; (vii) a CDR1, a CDR2, and a CDR3 having the amino acid sequences of the CDR1, CDR2, and CDR3, respectively, as set forth in SEQ ID NO:44; (viii) a CDR1, a CDR2, and a CDR3 having the amino acid sequences of the CDR1, CDR2, and CDR3, respectively, as set forth in SEQ ID NO:45; (ix) a CDR1, a CDR2, and a CDR3 having the amino acid sequences of the CDR1, CDR2, and CDR3, respectively, as set forth in SEQ ID NO:46; (x) a CDR1, a CDR2, and a CDR3 having the amino acid sequences of the CDR1, CDR2, and CDR3, respectively, as set forth in SEQ ID NO:47; (xi) a CDR1, a CDR2, and a CDR3 having the amino acid sequences of the CDR1, CDR2, and CDR3, respectively, as set forth in SEQ ID NO:48; (xii) a CDR1, a CDR2, and a CDR3 having the amino acid sequences of the CDR1, CDR2, and CDR3, respectively, as set forth in SEQ ID NO:49; (xiii) a CDR1, a CDR2, and a CDR3 having the amino acid sequences of the CDR1, CDR2, and CDR3, respectively, as set forth in SEQ ID NO:50; (xiv) a CDR1, a CDR2, and a CDR3 having the amino acid sequences of the CDR1, CDR2, and CDR3, respectively, as set forth in SEQ ID NO:51; (xv) a CDR1, a CDR2, and a CDR3 having the amino acid sequences of the CDR1, CDR2, and CDR3, respectively, as set forth in SEQ ID NO:77; (xvi) a CDR1, a CDR2, and a CDR3 having the amino acid sequences of the CDR1, CDR2, and CDR3, respectively, as set forth in SEQ ID NO:78; (xvii) a CDR1, a CDR2, and a CDR3 having the amino acid sequences of the CDR1, CDR2, and CDR3, respectively, as set forth in SEQ ID NO:79; (xviii) a CDR1, a CDR2, and a CDR3 having the amino acid sequences of the CDR1, CDR2, and CDR3, respectively, as set forth in SEQ ID NO:80; (xix) a CDR1, a CDR2, and a CDR3 having the amino acid sequences of the CDR1, CDR2, and CDR3, respectively, as set forth in SEQ ID NO:81; (xx) a CDR1, a CDR2, and a CDR3 having the amino acid sequences of the CDR1, CDR2, and CDR3, respectively, as set forth in SEQ ID NO:82; (xxi) a CDR1, a CDR2, and a CDR3 having the amino acid sequences of the CDR1, CDR2, and CDR3, respectively, as set forth in SEQ ID NO:83; or (xxii) a CDR1, a CDR2, and a CDR3 having the amino acid sequences of the CDR1, CDR2, and CDR3, respectively, as set forth in SEQ ID NO:84; or (xxiii) a CDR1, a CDR2, and a CDR3 having the amino acid sequences of the CDR1, CDR2, and CDR3, respectively, as set forth in SEQ ID NO:85. In some embodiments, the CDR1, CDR2 or CDR3 are determined according to the Kabat numbering scheme, the IMGT numbering scheme, the AbM numbering scheme, the Chothia numbering scheme, the Contact numbering scheme, or a combination thereof.

In some embodiments, the binding moiety further comprises one or more FR regions as set forth in SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:77, SEQ ID NO:78, SEQ ID NO:79, SEQ ID NO:80, SEQ ID NO:81, SEQ ID NO:82, SEQ ID NO:83, SEQ ID NO:84, and/or SEQ ID NO:85.

In some embodiments, the Claudin18.2 binding moiety comprises a single domain antibody or an antigen binding fragment comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to any one of SEQ ID NOs:38-51 and 77-85. In some embodiments, the Claudin18.2 binding moiety is a single domain antibody. In some embodiments, the Claudin18.2 binding moiety is a VHH domain. In some embodiment, the Claudin18.2 binding moiety is a heavy chain only antibody (HCAb), and the HCAb comprises a VHH domain having an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to any one of SEQ ID NOs:38-51 and 77-85.

Provided herein is also a binding moiety that competes with the binding moiety described above for Claudin18.2 binding. The exemplary Claudin18.2 binding moiety disclosed herein binds Claudin18.2 with an affinity that is at least 50 fold greater than its affinity to Claudin18.1.

In some embodiments, the binding moiety disclosed herein comprises at least two anti-Claudin18.2 single domain antibodies or antigen binding fragments (such as VHH fragments) connected by linker(s), wherein the anti-Claudin18.2 single domain antibodies or antigen binding fragments bind to a same antigen epitope. In some embodiments, the binding moiety comprises at least two anti-Claudin18.2 single domain antibodies or antigen-binding fragments (such as VHH fragments) connected by linker(s), wherein these single domain antibodies or antigen binding fragments bind to different antigen epitopes.

In some embodiments, the binding moiety described herein comprises a constant region, linked to the C-terminus of the variable region, e.g., the VHH domain/fragment. In some embodiments, the constant region is an immunoglobin heavy chain constant region or a portion of an immunoglobin heavy chain constant region, such as a hinge-CH2-CH3 domain of an immunoglobin heavy chain constant region. In some embodiments, the constant region of the disclosure is an IgG, IgM, or IgA heavy chain constant region or a portion thereof. In some embodiments, the constant region is an IgG1, IgG2 or IgG4 heavy chain constant region, or a portion thereof, such as a hinge-CH2-CH3 domain of an IgG1, IgG2 or IgG4 heavy chain constant region. In some embodiments, the constant region of the disclosure is a hinge-CH2-CH3 domain of a human or camelid IgG1, IgG2 or IgG4 heavy chain constant region. In one embodiment, the constant region is a human IgG1 constant region having an amino acid sequence set forth in SEQ ID NO:76.

In some embodiments, the binding moiety provided herein is a camelid, chimeric, human or humanized single domain antibody, or an antigen binding fragment thereof.

The present disclosure also provides a binding moiety comprising (i) an anti-Claudin18.2 single domain antibody or an antigen binding fragment, and (ii) an antibody light chain or a portion thereof, which two are linked by disulfide bonds to bind Claudin18.2. In some embodiment, the binding moiety comprises a Fab, a Fab′, a F(ab′)₂, a Fv, a scFv, a (scFv)₂, an IgG1 antibody, an IgG2 antibody, an IgG3 antibody, or an IgG4 antibody.

In some embodiments, the single domain antibody or antigen binding fragment thereof provided herein is genetically fused or chemically conjugated to an agent. The present disclosure also provides an immunoconjugate comprising a Claudin18.2 binding moiety described herein linked to a therapeutic agent, such as a cytotoxin. The present disclosure further provides a bispecific molecule comprising a Claudin18.2 binding moiety described herein, linked to a second functional moiety (e.g., a second binding moiety) having a different binding specificity with said Claudin18.2 binding moiety, including a second binding moiety binding to a different Claudin18.2 epitope, or a second binding moiety binding to a different antigen. Also provided is a multispecific molecule comprising a Claudin18.2 binding moiety described herein, linked to two or more functional moieties (e.g., two or more binding moieties) having different binding specificities than said Claudin18.2 binding moiety, such as a second binding moiety binding to a different Claudin18.2 epitope or a different antigen, and a third binding moiety binding to a different Claudin18.2 epitope or a different antigen. In some embodiments, the Claudin18.2 binding moiety described herein is expressed by or used in conjunction with an oncolytic virus.

The present disclosure also provides a polynucleotide encoding the Claudin18.2 binding moiety described herein, a vector comprising the polynucleotide, and a host cell containing the vector. In some embodiments, the vector is an expression vector. In some embodiments, the vector is a viral vector. In some embodiments, the vector is a lentiviral vector. In some embodiments, the vector is a non-viral vector.

In another aspect, the present application provides a chimeric antigen receptor (CAR) comprising a Claudin18.2 binding moiety described herein. In some embodiments, the Claudin18.2 CAR comprises (a) an extracellular antigen binding domain comprising a Claudin18.2 binding moiety described herein; (b) a transmembrane domain; and (c) an intracellular signaling domain.

In some embodiments, the Claudin18.2 CAR comprises (a) an extracellular antigen binding domain comprising an anti-Claudin18.2 single domain antibody or an antigen binding fragment thereof described herein such as a VHH domain; (b) a transmembrane domain; and (c) an intracellular signaling domain.

In some embodiments, the Claudin18.2 CAR comprises a Claudin18.2 binding moiety comprising one or more anti-Claudin18.2 single domain antibodies or antigen binding fragments such as VHH domains in the extracellular antigen binding domain. In some embodiments, the Claudin18.2 CAR comprises one or more anti-Claudin18.2 single domain antibodies or antigen binding fragments connected by linker(s), wherein these single domain antibodies or antigen binding fragments bind to a same antigen epitope. In some embodiments, the Claudin18.2 CAR comprises one or more anti-Claudin18.2 single domain antibodies or antigen binding fragments connected by linker(s), wherein these single domain antibodies or antigen binding domains bind to different antigen epitopes. In some embodiments, the Claudin18.2 CAR comprises at least two VHH domains connected by linker(s), wherein the VHH domains bind to a same antigen epitope. In some embodiments, the Claudin18.2 CAR comprises at least two VHH domains connected by linker(s), wherein these VHH domains bind to different antigen epitopes.

In some embodiments, the extracellular antigen binding domain further comprises one or more additional antigen binding domain(s). In some embodiments, the extracellular antigen binding domain further comprises one additional antigen binding domain. In other embodiments, the extracellular antigen binding domain further comprises two additional antigen binding domains.

In some embodiments, the Claudin18.2 CAR further comprises a signal peptide located at the N-terminus. In some embodiments, the signal peptide is derived from a molecule selected from the group consisting of CD8α, GM-CSF receptor α, and IgG1 heavy chain. In some embodiments, the signal peptide is derived from CD8α. In some embodiments, the signal peptide comprises an amino acid sequence of SEQ ID NO:67.

In some embodiments, the Claudin18.2 CAR further comprise a hinge domain located between the C-terminus of the extracellular antigen binding domain and the N-terminus of the transmembrane domain. In some embodiments, the hinge domain is derived from CD8α In some embodiments, the hinge domain comprises an amino acid sequence of SEQ ID NO:68.

In some embodiments according to any one of the CARs described above, the transmembrane domain is derived from a molecule selected from the group consisting of CD8α, CD4, CD28, CD137, CD80, CD86, CD152 and PD1. In some embodiments, the transmembrane domain is derived from CD8α or CD28. In some embodiments, the transmembrane domain comprises an amino acid sequence of SEQ ID NO:69.

The intracellular signaling domain comprises a primary intracellular signaling domain and/or a co-stimulatory signaling domain. In some embodiments, the intracellular signaling domain comprises a primary intracellular signaling domain of an immune effector cell (such as a T cell). In some embodiments, the primary intracellular signaling domain is an immunoreceptor tyrosine-based activation motif (ITAM)-containing domain. In some embodiments, the ITAM-containing domain is CD3-zeta’s cytoplasmic domain comprising an amino acid sequence of SEQ ID NO:72. In some embodiments, the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 (such as CD83 and MD-2) and combinations thereof. In some embodiments, the co-stimulatory signaling domain comprises a cytoplasmic domain of CD28 and/or a cytoplasmic domain of CD137. In some embodiments, the cytoplasmic domain of CD28 and the cytoplasmic domain of CD137 comprise amino acid sequences of SEQ ID NO:71 and SEQ ID NO:70, respectively.

In some embodiments, the Claudin18.2 CAR comprises, from N-terminus to C-terminus, a signal peptide, a Claudin18.2 binding moiety comprising an anti-Claudin18.2 single domain antibody or an antigen binding fragment thereof described herein such a VHH domain, a hinge domain, a transmembrane domain, a primary intracellular signaling domain and/or a co-stimulatory signaling domain. In some embodiments, the CAR comprises, from N-terminus to C-terminus, a signal peptide derived from CD8α, an anti-Claudin18.2 VHH domain, a hinge domain derived from CD8α, a transmembrane domain derived from CD8α or CD28, a CD137 cytoplasmic domain, and a CD3-zeta’s cytoplasmic domain In some embodiments, the CAR comprises, from N-terminus to C-terminus, a signal peptide of SEQ ID NO:67, a VHH domain described above having an amino acid sequence selected from the group of SEQ ID NOs:38-51 and 77-85, a hinge domain of SEQ ID NO:68, a transmembrane domain of SEQ ID NO:69, a CD137 cytoplasmic domain of SEQ ID NO:70, and a CD3-zeta’s cytoplasmic domain of SEQ ID NO:72.

In some embodiments, the CAR comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence of any one of SEQ ID NOs: 53-66 and 86-93. In some embodiments, the CAR comprises an amino acid sequence of any one of SEQ ID NOs: 53-66 and 86-93.

The present application provides a nucleic acid encoding the CAR described herein. The present application also provides a vector comprising the nucleic acid described above. In some embodiments, the vector is an expression vector. In some embodiments, the vector is a viral vector, a lentiviral vector or a non-viral vector.

The present application provides an engineered immune cell, comprising the CAR described above, or the nucleic acid described above, or the vector described above. In some embodiments, the immune cell is an immune effector cell, such as a T cell, an NK cell, a peripheral blood mononuclear cell (PBMC), a hematopoietic stem cell, a pluripotent stem cell, or an embryonic stem cell. In some embodiments, the immune cell is a T cell, such as a cytotoxic T cell, a helper T cell, a natural killer T cell, or a γδT cell.

The present application is further directed to a pharmaceutical composition comprising a therapeutically effective amount of the binding moiety, the immunoconjugate, the bispecific molecule, the multispecific or multivalent molecule, the oncolytic virus, the CAR and/or the engineered immune cell described herein, and a pharmaceutically acceptable excipient.

Also provided herein is a method of treating a Claudin18.2-expressing tumor or cancer in a subject in need thereof, by administering to the subject a therapeutically effective amount of the pharmaceutical composition described herein.

The Claudin18.2-expressing tumor or cancer is a solid or non-solid tumor or cancer, including, but not limited to, gastric, esophageal, gastroesophageal, pancreatic, ovarian, colon, hepatic, head-neck, gallbladder and lung tumor or cancer. In some embodiments, the Claudin18.2-expressing tumor or cancer is a gastric tumor or cancer. In some embodiments, the Claudin18.2-expressing tumor or cancer is a gastroesophageal tumor or cancer. In some embodiments, the subject is human.

In some embodiments, the engineered immune cell for treating the tumor or cancer is autologous. In some embodiments, the engineered immune cell is allogenic.

Also provided are methods of use, kits, and articles of manufacture comprising any one of the Claudin18.2 binding moieties, CARs, engineered immune effector cells, isolated nucleic acids, or vectors described above.

### 4. BRIEF DESCRIPTION OF THE FIGURES

FIG. 1 shows binding potencies of chimeric anti-Claudin18.2 antibodies of the disclosure on PANC1.huCLDN18.1.Luc and PANC1.huCLDN18.2.Luc cells.

FIG. 2 shows in vitro cytotoxicity of T cells carrying LIC182501-LIC182510 CARs against Claudin18.2 positive or negative cell lines. “UnT” indicates untransduced T cells that serve as control.

FIG. 3 shows in vitro cytotoxicity of T cells carrying LIC182511-LIC182514 CARs against Claudin18.2 positive or negative cell lines. “UnT” indicates untransduced T cells that serve as control.

FIG. 4 shows in vivo anti-tumor efficacy of Claudin18.2 CAR-T cells in a NUGC4 cell engrafted xenograft model. Mice were assessed to monitor tumor growth by the changes in tumor volume (part a) and the endpoint tumor weight (part b). **** indicates p<0.0001; ** indicates 0.001<p<0.01; * indicates 0.01<p<0.05.

FIG. 5 shows binding characteristic of humanized anti-Claudin18.2 chimeric antibodies. The chimeric antibodies showed potent binding to PANC1.huCLDN18.2.Luc in a dose dependent manner, but not to PANC1.huCLDN18.1.Luc cells. “CLDN18.2” indicates PANC1.huCLDN18.2.Luc cells; “CLDN18.1” indicates PANC1.huCLDN18.1.Luc cells.

FIG. 6 shows results of an in vitro cytotoxicity assay of humanized Claudin18.2 CAR-T cells as well as their parental CAR-T cells against PANC1.huCLDN18.2.Luc cell line, PANC1.huCLDN18.1.Luc cell line, and NUGC4.Luc cell line, respectively. 175DX CAR-T serves as benchmark control. CD19 CAR-T serves as negative control. “UnT” indicates untransduced T cells that serve as control.

FIG. 7 shows IFNy release of humanized Claudin18.2 CAR-T cells as well as their parental CAR-T cells co-culture with PANC1.huCLDN18.2.Luc cell line, PANC1.huCLDN18.1.Luc cell line, and NUGC4.Luc cell line, respectively. Part d shows spontaneous IFN_{γ} release of CAR-T cells. 175DX CAR-T serves as benchmark control. CD19 CAR-T serves as negative control. “UnT” indicates untransduced T cells that serve as control.

### 5. DETAILED DESCRIPTION

The present disclosure is based in part on the novel single domain antibodies (e.g., VHH domains) that bind to Claudin18.2, chimeric antigen receptors or engineered cells comprising same, and improved properties thereof.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “binding moiety” as used herein refers to a molecule or a portion of a molecule which specifically binds an antigen such as Claudin18.2. The binding moiety may be a protein, a peptide, a nucleic acid, a carbohydrate, a lipid, or a small molecular weight compound. In some embodiments, the binding moiety comprises an antibody or an antigen binding fragment thereof. In some embodiments, the binding moiety is an antibody or an antigen binding fragment thereof. The Claudin18.2 binding moiety also includes receptors, ligands, aptamers, and other molecules having a known binding partner. A binding moiety may be monovalent, which means that it contains one binding site that specifically interacts with an antigen such as Claudin18.2. A binding moiety may also be bivalent, meaning that it contains two binding sites that specifically interact with an antigen such as Claudin18.2. A binding moiety may be multivalent, meaning that is contains multiple binding sites that specifically interact with an antigen such as Claudin18.2. A bivalent binding moiety or a multivalent binding moiety may interact with one or more epitopes on a single antigen such as Claudin18.2 molecule. In some embodiments, a bivalent binding moiety or multivalent binding moiety may interact with two or more Claudin18.2 molecules.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The term “heavy chain-only antibody” or “HCAb″refers to a functional antibody, which comprises heavy chains, but lacks the light chains usually found in 4-chain antibodies. Camelid animals (such as camels, llamas, or alpacas) are known to produce HCAbs.

“Single domain antibody” or “sdAb” as used herein refers to a single monomeric variable antibody domain and which is capable of antigen binding (e.g., single domain antibodies that bind to Claudin18.2). Single domain antibodies include VHH domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, dromedary, alpaca and guanaco, as described herein. Other species besides Camelidae may produce heavy chain antibodies naturally devoid of light chain; VHHs derived from such other species are within the scope of the disclosure. In some embodiments, the single domain antibody (e.g., VHH) provided herein has a structure of FR1-CDR1-FR2-CDR2-FR3-CDR3-FR4. Single domain antibodies may be genetically fused or chemically conjugated to another molecule (e.g., an agent) as described herein. Single domain antibodies may be part of a bigger binding molecule (e.g., a multispecific antibody or a chimeric antigen receptor).

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis or RIA. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (K_{D}) of less than or equal to 1 µM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species

The term “EC₅₀”, also known as half maximal effective concentration, refers to the concentration of an antibody which induces a response halfway between the baseline and maximum after a specified exposure time.

The term “IC₅₀”, also known as half maximal inhibitory concentration, refers to the concentration of an antibody which inhibits a specific biological or biochemical function by 50% relative to the absence of the antibody.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos: 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise a single domain antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1: 755-68 (2006)). Also available for the preparation of human monoclonal antibodies are methods described in Cole et al., Monoclonal Antibodies and Cancer Therapy 77 (1985); Boerner et al., J. Immunol. 147(1):86-95 (1991); and van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6(5):561-66 (1995); Bruggemann and Taussing, Curr. Opin. Biotechnol. 8(4):455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid iomerizatio or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and _{γ} chains and four CH domains for µ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The amino acid residues of a single domain antibody (e.g., VHH) are numbered according to the general numbering for VH domains given by Kabat et al. (“Sequence of proteins of immunological interest”, US Public Health Services, NIH Bethesda, Md., Publication No. 91), as applied to VHH domains from Camelids in the article of Riechmann and Muyldermans, J. Immunol. Methods 2000 Jun. 23; 240 (1-2): 185-195. According to this numbering, FR1 of a VHH comprises the amino acid residues at positions 1-30, CDR1 of a VHH comprises the amino acid residues at positions 31-35, FR2 of a VHH comprises the amino acids at positions 36-49, CDR2 of a VHH comprises the amino acid residues at positions 50-65, FR3 of a VHH comprises the amino acid residues at positions 66-94, CDR3 of a VHH comprises the amino acid residues at positions 95-102, and FR4 of a VHH comprises the amino acid residues at positions 103-113. In this respect, it should be noted that—as is well known in the art for VH domains and for VHH domains—the total number of amino acid residues in each of the CDR’s may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). See, e.g., Deschacht et al., 2010. J Immunol 184: 5696-704 for an exemplary numbering for VHH domains according to Kabat.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (µ), based on the amino acid sequence of the heavy chain constant region. The distinct heavy chains differ in size: α, δ, and _{γ} contain approximately 450 amino acids, while µ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular’s AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27(1):55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in Table 1 below.

TABLE 1

Exemplary CDRs According to Various Numbering Systems

| | | | | | |
|---|---|---|---|---|---|
| Loop | Kabat | AbM | Chothia | Contact | IMGT |
| CDR L1 | L24--L34 | L24--L34 | L26--L32 or L24--L34 | L30--L36 | L27--L38 |
| CDR L2 | L50--L56 | L50--L56 | L50--L52 or L50--L56 | L46--L55 | L56--L65 |
| CDR L3 | L89--L97 | L89--L97 | L91--L96 or L89--L97 | L89--L96 | L105-L117 |
| CDR H1 | H31--H35B (Kabat Numbering) | H26--H35B | H26--H32.0.34 | H30--H35B | H27-H38 |
| CDR H1 | H31--H35 (Chothia Numbering) | H26--H35 | H26--H32 | H30--H35 | |
| CDR H2 | H50--H65 | H50--H58 | H53--H55 or H52--H56 | H47--H58 | H56--H65 |
| CDR H3 | H95--HI02 | H95--H102 | H96-H101 or H95--H102 | H93-H101 | H105-H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR1 as set forth in a specific VH or VHH” includes any CDR1 as defined by the exemplary CDR numbering systems described above, but is not limited thereby. Once a variable region (e.g., a VHH, VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies (e.g., single domain antibodies), diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody comprising a single domain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

A “blocking” antibody or an “antagonist” antibody is one that inhibits or reduces a biological activity of the antigen it binds. In some embodiments, blocking antibodies or antagonist antibodies substantially or completely inhibit the biological activity of the antigen.

An “agonist” or activating antibody is one that enhances or initiates signaling by the antigen to which it binds. In some embodiments, agonist antibodies cause or activate signaling without the presence of the natural ligand.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “specificity” refers to selective recognition of an antigen binding protein (such as a CAR or an sdAb) for a particular epitope of an antigen. Natural antibodies, for example, are monospecific. The term “multispecific” as used herein denotes that an antigen binding protein (such as a CAR or an sdAb) has two or more antigen-binding sites of which at least two bind different antigens. “Bispecific” as used herein denotes that an antigen binding protein (such as a CAR or an sdAb) has two different antigen-binding specificities. The term “monospecific” CAR as used herein denotes an antigen binding protein (such as a CAR or an sdAb) that has one or more binding sites each of which bind the same antigen.

The term “valent” as used herein denotes the presence of a specified number of binding sites in an antigen binding protein (such as a CAR or an sdAb). A natural antibody for example or a full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein (such as a CAR or an sdAb).

“Chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” In some embodiments, the CAR comprises an extracellular antigen binding domain specific for one or more antigens (such as tumor antigens), a transmembrane domain, and an intracellular signaling domain of a T cell and/or other receptors. “CAR-T cell” refers to a T cell that expresses a CAR.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding a single domain antibody or an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding a CAR or an sdAb described herein is a nucleic acid molecule that is identified and separated from at least one contaminant nucleic acid molecule with which it is ordinarily associated in the environment in which it was produced.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell’s chromosome. Additionally, the vectors can include one or more selectable marker genes and appropriate expression control sequences Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “autologous” is meant to refer to any material derived from the same individual to whom it is later to be re-introduced into the individual.

“Allogeneic” refers to a graft derived from a different individual of the same species.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds’ adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington’s Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a binding molecule (e.g., an antibody), for example, in isolated or purified form, together with a suitable amount of excipients.

The term “effective amount” or “therapeutically effective amount” as used herein refers to the amount of a single domain antibody or a therapeutic molecule comprising an agent and the single domain antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., diabetes or a cancer).

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of′ are also provided. It is also understood that wherever embodiments are described herein with the phrase “consisting essentially of” otherwise analogous embodiments described in terms of “consisting of” are also provided

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2. Claudin18.2 Binding Moieties

Claudin18.2 is isoform 2 of Claudin18, a member of the Claudin family of cell surface proteins. Claudins are important components of the tight cell junctions, forming a paracellular barrier which controls the flow of molecules between the cells. Different claudins are expressed on different tissues, and their altered function has been linked to the formation of cancers of these tissues. In normal tissues, the expression of Claudin18.2 is limited to the epithelial cells of the gastric mucosa. Claudin18.2 expression is retained upon malignant transformation in gastric cancer and its metastases. Ectopic activation of Claudin18.2 has also been found in pancreatic, esophageal, ovarian, and lung tumors.

The human Claudin18.2 protein has 261 amino acids (NCBI, NP_001002026.1). Claudin18.2 is a tetraspan transmembrane protein, with an N-terminus and a C-terminus in the cytoplasm. Claudin18.2 has two extracellular loops, which have been linked to functions such as tightening of the paracellular cleft for solutes, and the formation paracellular ion pores.

The Claudin18.2 binding moiety provided herein specifically binds Claudin18.2, a fragment thereof, or a variant thereof. In some embodiments, a Claudin18.2 binding moiety specifically binds human Claudin18.2. In some embodiments, a Claudin18.2 binding moiety specifically binds an extracellular domain of Claudin18.2. In some embodiments, a Claudin18.2 binding moiety specifically binds the first extracellular loop of Claudin18.2. In some embodiments, a Claudin18.2 binding moiety specifically binds the second extracellular loop of Claudin18.2. In some embodiments, a Claudin18.2 binding moiety specifically binds both the first and the second extracellular loops of Claudin18.2. In some embodiments, the Claudin18.2 binding moiety binds Claudin18.2 with an affinity that is at least 20-fold greater than the antibody’s affinity to Claudin18.1. In some embodiments, the Claudin18.2 binding moiety binds Claudin18.2 with an affinity that is at least 50-fold greater than the antibody’s affinity to Claudin18.1. In some embodiments, the Claudin18.2 binding moiety binds Claudin18.2 with an affinity that is at least 100-fold greater than the antibody’s affinity to Claudin18.1. In some embodiments, the Claudin18.2 binding moiety does not detectably bind Claudin18.1.

In some embodiments, a Claudin18.2 binding moiety (e.g., an antibody) provided herein binds Claudin18.2 (e.g., human Claudin18.2) with a dissociation constant (K_{D}) of about 1.0 µM or less, about 100.0 nM or less, about 40.0 nM or less, about 20.0 nM or less, about 10.0 nM or less, about 1.0 nM or less, about 0.1 nM or less, 50.0 pM or less, 10.0 pM or less, or 1.0 pM or less. In some embodiments, a Claudin18.2 binding moiety (e.g., an antibody) binds Claudin18.2 (e.g., human Claudin18.2) with a half maximal effective concentration (EC₅₀) of about 1.0 µM or less, about 100.0 nM or less, about 40.0 nM or less, about 20.0 nM or less, about 10.0 nM or less, about 1.0 nM or less, or about 0.1 nM or less.

In some embodiments, the Claudin18.2 binding moiety provided herein modulates one or more Claudin18.2 activities. In some embodiments, the Claudin18.2 binding moiety provided herein is an antagonist antibody.

### 5.2.1. Single Domain Antibodies That Bind Claudin18.2

In some embodiments, the Claudin18.2 binding moiety comprises a single domain antibody or an antigen binding fragment thereof. In some embodiments, the single domain antibody is a heavy chain only antibody (HCAb), and the antigen binding fragment is a variable region of heavy chain-only antibody (HCAb), i.e., a VHH fragment, or a VHH fragment with a complete or partial heavy chain constant region. In some embodiments, the Claudin18.2 binding moiety comprises at least two VHH fragments connected by linker(s), wherein the VHH fragments bind to a same antigen epitope. In some embodiments, the Claudin18.2 binding moiety comprises at least two VHH fragments connected by linker(s), wherein these VHH fragments bind to different antigen epitopes. In some embodiments, the Claudin18.2 binding moiety comprises one or more VHH domains linked to a complete or partial heavy chain constant region. In some embodiments, the heavy chain constant region is an immunoglobin heavy chain constant region or a portion of an immunoglobin heavy chain constant region, such as a hinge-CH2-CH3 domain of an immunoglobin heavy chain constant region. In some embodiments, the heavy chain constant region of the disclosure is an IgG1, IgG2 or IgG4 heavy chain constant region, or a portion thereof, such as a hinge-CH2-CH3 domain of an IgG1, IgG2 or IgG4 heavy chain constant region. In some embodiments, heavy chain constant region of the disclosure is a hinge-CH2-CH3 domain of a human or camelid IgG1, IgG2 or IgG4 heavy chain constant region.

In some embodiments, the Claudin18.2 binding moiety provides a binding moiety comprising (i) the anti-Claudin18.2 single domain antibody or an antigen binding fragment and (ii) an antibody light chain or a portion thereof, which two are linked by disulfite bonds to bind Claudin18.2. In some embodiment, the binding moiety is a Fab, a Fab′, a F(ab′)₂, a Fv, a scFv, a (scFv)₂, an IgG1 antibody, an IgG2 antibody, an IgG3 antibody, or an IgG4 antibody.

In some embodiments, the binding moiety provided herein is a camelid, chimeric, human or humanized single domain antibody, or an antigen binding fragment thereof.

In some embodiments, the antibody is isolated. In some embodiments, the antibody is substantially pure.

In some embodiments, a Claudin18.2 binding moiety is a monospecific binding moiety. In some embodiments, a Claudin18.2 binding moiety is a bispecific binding moiety. In some embodiments, a Claudin18.2 binding moiety is a multispecific binding moiety.

In some embodiments, a Claudin18.2 binding moiety comprises a monovalent binding moiety. In some embodiments, a Claudin18.2 binding moiety comprises a bivalent binding moiety. In some embodiments, a Claudin18.2 binding moiety comprises a multivalent binding moiety. In some embodiments, the bivalent binding moiety comprises two single domain antibodies or antigen binding fragments thereof. In some embodiments, the bivalent binding moiety comprises a first single domain antibody or an antigen binding fragment thereof and a second single domain antibody or an antigen binding fragment thereof. In some embodiments, the first single domain antibody or antigen binding fragment is linked to the second single domain antibody or antigen binding fragment by a linker. In some embodiments, a Claudin18.2 binding moiety comprises a first single domain antibody or an antigen binding fragment thereof, a linker and a second single domain antibody or an antigen binding fragment thereof, from N-terminus to C-terminus. In some embodiments, the second single domain antibody or antigen binding fragment thereof is a tandem repeat of the first single domain antibody or antigen binding fragment thereof. In some embodiments, the first single domain antibody or antigen binding fragment thereof and the second single domain antibody or antigen binding fragment thereof recognize different epitopes on Claudin18.2. In some embodiments, the first single domain antibody or antigen binding fragment and the second single domain antibody or antigen binding fragment recognize the same epitope on Claudin18.2.

The antibodies (such as sdAbs) described herein may be prepared using any methods known in the art or as described in more detail below (e.g., in Section 5.2.6).

In some embodiments, a Claudin18.2 binding moiety is a humanized antibody. Various methods for generating humanized antibodies are known in the art and as described in more detail below (e.g., in Section 5.2.2). In some embodiments, a humanized antibody comprises one or more amino acid residues that have been introduced into its sequence from a source that is non-human.

The CDRs of an antibody are defined by those skilled in the art using a variety of methods/systems. These systems and/or definitions have been developed and refined over a number of years and include Kabat, Chothia, IMGT, AbM, and Contact The Kabat definition is based on sequence variability and is commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. In some embodiments, the exemplary CDRs according various systems are defined as in Table 1 above. However, it will be understood that reference to a variable domain CDR or CDRs of a specific antibody will encompass all CDR definitions as known to those of skill in the art.

The Claudin18.2 binding moieties provided herein include anti-Claudin18.2 single domain antibodies provided herein, and humanized versions thereof, and antigen binding fragments thereof, with CDR1, CDR2, CDR3 defined according to Kabat numbering referring to Deschacht et al., 2010. J Immunol 184: 5696-704 and variable region sequences or sequence ID numbers summarized in Table 2 below.

In some embodiments, a Claudin18.2 binding moiety is an anti-Claudin18.2 single domain antibody that comprises one, two, and/or three CDRs of any one of the antibodies described herein. In some embodiments, an anti-Claudin18.2 single domain antibody comprises one, two, and/or three CDRs, or the variable region from Table 2. In some embodiments, the anti-Claudin18.2 single domain antibody contains a constant region linked to the C-terminus of the VHH domain.

Exemplary anti-Claudin18.2 antibodies of the disclosure show higher binding capacity to Claudin18.2 as compared to a benchmark containing a single chain variable fragment (scFv) with IMAB362′s heavy chain variable region (V_{H}) and light chain variable region (V_{L}).

TABLE 2

Amino acid sequences and/or sequence ID number of CDRs and VHH domains

| | | | | |
|---|---|---|---|---|
| Clone ID | VHH domain (SEQ ID NO.) | CDR1 | CDR2 | CDR3 |
| VHH182501 | SEQ ID NO:38 | RYGMT (SEQ ID NO:1) | SISISGSTEYADSVK G (SEQ ID NO:12) | YNAYTQY (SEQ ID NO:24) |
| VHH182502 | SEQ ID NO:39 | AKCMA (SEQ ID NO:2) | TIQSGYYRPYYTDS VKG (SEQ ID NO: 13) | DRFTYPPGVICGGI SENYRY (SEQ ID NO:25) |
| VHH182503 | SEQ ID NO:40 | AYYMG (SEQ ID NO:3) | VIPGGTQTYYADSV KG(SEQ ID NO:14) | TYLVGSAPLSPSRF RY (SEQ ID NO:26) |
| VHH 182504 | SEQ ID NO:41 | LYCMG (SEQ ID NO:4) | AIDYYGSTTYADSV KG (SEQ ID NO:15) | RRTSWCSVSFNGY DD (SEQ ID NO:27) |
| VHH182505 | SEQ ID NO:42 | SVCMG (SEQ ID NO:5) | LIGRTGSTTYIDSV KG(SEQ ID NO:16) | GLGYWDCDYNY( SEQ ID NO:28) |
| VHH182506 | SEQ ID NO:43 | SGDMN (SEQ ID NO:6) | GINRAGNTNYAAS VKG (SEQ ID NO: 17) | TYCRMALFGACN DY (SEQ ID NO:29) |
| VHH182507 | SEQ ID NO:44 | CNYMS (SEQ ID NO:7) | RIFPSGKTTYADSM KA (SEQ ID NO:18) | SCRGPGSIWPSDY (SEQ ID NO:30) |
| VHH182508 | SEQ ID NO:45 | SYCMS (SEQ ID NO:8) | AISTRGGSTYYVDS VKG (SEQ ID NO:19) | AEGLCGWGGWES AVRFGS (SEQ ID NO:31) |
| VHH182509 | SEQ ID NO:46 | RYGMT (SEQ ID NO:1) | SISLSGSTEYADSV KG (SEQ ID NO:20) | YNAYGQY (SEQ ID NO:32) |
| VHH182510 | SEQ ID NO:47 | SVCMG (SEQ ID NO:5) | VIGRDGSTTYIDSV KG (SEQ ID NO:21) | GLGYWACEYDY(S EQ ID NO:33) |
| VHH182511 | SEQ ID NO:48 | TYCMG (SEQ ID NO:9) | NVYMRNGDTWLA DSVKG (SEQ ID NO:22) | GRSSCLEASISY (SEQ ID NO:34) |
| VHH182512 | SEQ ID NO:49 | TANMA (SEQ ID NO:10) | AAHSDGSTKYADS VKG (SEQ ID NO:23) | RGSPWWNSVVAL ESYAYSY (SEQ ID NO:35) |
| VHH182513 | SEQ ID NO:50 | SVCMG (SEQ ID NO:5) | VIGRDGSTTYIDSV KG(SEQ ID NO:21) | GLGYWACEYNY(S EQ ID NO:36) |
| VHH182514 | SEQ ID NO 51 | SKCMA (SEQ ID NO: 11) | TIQSGYYRPYYTDS VKG (SEQ ID NO: 13) | DRFTYPYGVICGGI SANYRY (SEQ ID NO:37) |
| VHH182513 H1 | SEQ ID NO:77 | SVCMG (SEQ ID NO:5) | VIGRDGSTTYIDSV KG(SEQ ID NO:21) | GLGYWACEYNY(S EQ ID NO:36) |
| VHH182513 H4 | SEQ ID NO:78 | SVCMG (SEQ ID NO:5) | VIGRDGSTTYIDSV KG(SEQ ID NO:21) | GLGYWACEYNY(S EQ ID NO:36) |
| VHH182513 H7 | SEQ ID NO:79 | SVCMG (SEQ ID NO:5) | VIGRDGSTTYIDSV KG(SEQ ID NO:21) | GLGYWACEYNY(S EQ ID NO:36) |
| VHH182513 H8 | SEQ ID NO:80 | SVCMG (SEQ ID NO:5) | VIGRDGSTTYIDSV KG(SEQ ID NO:21) | GLGYWACEYNY(S EQ ID NO:36) |
| VHH182513 H9 | SEQ ID NO:81 | SVCMG (SEQ ID NO:5) | VIGRDGSTTYIDSV KG(SEQ ID NO:21) | GLGYWACEYNY(S EQ ID NO:36) |
| VHH182513 H10 | SEQ ID NO:82 | SVCMG (SEQ ID NO:5) | VIGRDGSTTYIDSV KG(SEQ ID NO:21) | GLGYWACEYNY(S EQ ID NO:36) |
| VHH182511 H6 | SEQ ID NO:83 | TYCMG (SEQ ID NO:9) | NVYMRNGDTWLA DSVKG (SEQ ID NO:22) | GRSSCLEASISY (SEQ ID NO:34) |
| VHH182511 H8 | SEQ ID NO:84 | TYCMG (SEQ ID NO:9) | NVYMRNGDTWLA DSVKG (SEQ ID NO:22) | GRSSCLEASISY (SEQ ID NO:34) |
| VHH182511 H9 | SEQ ID NO: 85 | TYCMG (SEQ ID NO:9) | NVYMRNGDTWLA DSVKG (SEQ ID NO:22) | GRSSCLEASISY (SEQ ID NO:34) |

One aspect of the present application provides an anti-Claudin18.2 sdAb comprising the CDR regions of any one of SEQ ID NOs: 38-51 and 77-85. Thus, in some embodiments, provided herein is a single domain antibody that binds to Claudin18.2 comprising the following structure: FR1-CDR1-FR2-CDR2-FR3-CDR3-FR4, wherein the CDR sequences are selected from those in the single domain antibodies in Table 2.

In some embodiments, there is provided an anti-Claudin18.2 single domain antibody comprising one, two, or all three CDRs of the amino acid sequence of any one of SEQ ID NOs: 38-51 and 77-85. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 38. In some embodiments, the single domain antibody has a CDR2 having an amino acid sequence of the CDR2 as set forth in SEQ ID NO:38. In other embodiments, the single domain antibody has a CDR3 having an amino acid sequence of the CDR3 as set forth in SEQ ID NO:38. In some embodiments, the single domain antibody has a CDR1 and a CDR2 having amino acid sequences of the CDR1 and the CDR2 as set forth in SEQ ID NO:38. In some embodiments, the single domain antibody has a CDR1 and a CDR3 having amino acid sequences of the CDR1 and the CDR3 as set forth in SEQ ID NO:38. In some embodiments, the single domain antibody has a CDR2 and a CDR3 having amino acid sequences of the CDR2 and the CDR3 as set forth in SEQ ID NO:38. In some embodiments, the single domain antibody has a CDR1, a CDR2, and a CDR3 having amino acid sequences of the CDR1, the CDR2, and the CDR3 as set forth in SEQ ID NO:38. CDR sequences can be determined according to well-known numbering systems. In some embodiments, the CDRs are according to IMGT numbering. In some embodiments, the CDRs are according to Kabat numbering. In some embodiments, the CDRs are according to AbM numbering In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:39. In some embodiments, the single domain antibody has a CDR2 having an amino acid sequence of the CDR2 as set forth in SEQ ID NO:39. In other embodiments, the single domain antibody has a CDR3 having an amino acid sequence of the CDR3 as set forth in SEQ ID NO:39. In some embodiments, the single domain antibody has a CDR1 and a CDR2 having amino acid sequences of the CDR1 and the CDR2 as set forth in SEQ ID NO:39. In some embodiments, the single domain antibody has a CDR1 and a CDR3 having amino acid sequences of the CDR1 and the CDR3 as set forth in SEQ ID NO:39. In some embodiments, the single domain antibody has a CDR2 and a CDR3 having amino acid sequences of the CDR2 and the CDR3 as set forth in SEQ ID NO:39. In some embodiments, the single domain antibody has a CDR1, a CDR2, and a CDR3 having amino acid sequences of the CDR1, the CDR2, and the CDR3 as set forth in SEQ ID NO:39. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:40. In some embodiments, the single domain antibody has a CDR2 having an amino acid sequence of the CDR2 as set forth in SEQ ID NO:40. In other embodiments, the single domain antibody has a CDR3 having an amino acid sequence of the CDR3 as set forth in SEQ ID NO:40. In some embodiments, the single domain antibody has a CDR1 and a CDR2 having amino acid sequences of the CDR1 and the CDR2 as set forth in SEQ ID NO:40. In some embodiments, the single domain antibody has a CDR1 and a CDR3 having amino acid sequences of the CDR1 and the CDR3 as set forth in SEQ ID NO:40. In some embodiments, the single domain antibody has a CDR2 and a CDR3 having amino acid sequences of the CDR2 and the CDR3 as set forth in SEQ ID NO:40. In some embodiments, the single domain antibody has a CDR1, a CDR2, and a CDR3 having amino acid sequences of the CDR1, the CDR2, and the CDR3 as set forth in SEQ ID NO:40. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:41. In some embodiments, the single domain antibody has a CDR2 having an amino acid sequence of the CDR2 as set forth in SEQ ID NO:41. In other embodiments, the single domain antibody has a CDR3 having an amino acid sequence of the CDR3 as set forth in SEQ ID NO:41. In some embodiments, the single domain antibody has a CDR1 and a CDR2 having amino acid sequences of the CDR1 and the CDR2 as set forth in SEQ ID NO:41. In some embodiments, the single domain antibody has a CDR1 and a CDR3 having amino acid sequences of the CDR1 and the CDR3 as set forth in SEQ ID NO:41. In some embodiments, the single domain antibody has a CDR2 and a CDR3 having amino acid sequences of the CDR2 and the CDR3 as set forth in SEQ ID NO:41. In some embodiments, the single domain antibody has a CDR1, a CDR2, and a CDR3 having amino acid sequences of the CDR1, the CDR2, and the CDR3 as set forth in SEQ ID NO:41. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:42. In some embodiments, the single domain antibody has a CDR2 having an amino acid sequence of the CDR2 as set forth in SEQ ID NO:42. In other embodiments, the single domain antibody has a CDR3 having an amino acid sequence of the CDR3 as set forth in SEQ ID NO:42. In some embodiments, the single domain antibody has a CDR1 and a CDR2 having amino acid sequences of the CDR1 and the CDR2 as set forth in SEQ ID NO:42. In some embodiments, the single domain antibody has a CDR1 and a CDR3 having amino acid sequences of the CDR1 and the CDR3 as set forth in SEQ ID NO:42. In some embodiments, the single domain antibody has a CDR2 and a CDR3 having amino acid sequences of the CDR2 and the CDR3 as set forth in SEQ ID NO:42. In some embodiments, the single domain antibody has a CDR1, a CDR2, and a CDR3 having amino acid sequences of the CDR1, the CDR2, and the CDR3 as set forth in SEQ ID NO:42. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:43. In some embodiments, the single domain antibody has a CDR2 having an amino acid sequence of the CDR2 as set forth in SEQ ID NO:43. In other embodiments, the single domain antibody has a CDR3 having an amino acid sequence of the CDR3 as set forth in SEQ ID NO:43. In some embodiments, the single domain antibody has a CDR1 and a CDR2 having amino acid sequences of the CDR1 and the CDR2 as set forth in SEQ ID NO:43. In some embodiments, the single domain antibody has a CDR1 and a CDR3 having amino acid sequences of the CDR1 and the CDR3 as set forth in SEQ ID NO:43. In some embodiments, the single domain antibody has a CDR2 and a CDR3 having amino acid sequences of the CDR2 and the CDR3 as set forth in SEQ ID NO:43. In some embodiments, the single domain antibody has a CDR1, a CDR2, and a CDR3 having amino acid sequences of the CDR1, the CDR2, and the CDR3 as set forth in SEQ ID NO:43. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:44. In some embodiments, the single domain antibody has a CDR2 having an amino acid sequence of the CDR2 as set forth in SEQ ID NO:44. In other embodiments, the single domain antibody has a CDR3 having an amino acid sequence of the CDR3 as set forth in SEQ ID NO:44. In some embodiments, the single domain antibody has a CDR1 and a CDR2 having amino acid sequences of the CDR1 and the CDR2 as set forth in SEQ ID NO:44. In some embodiments, the single domain antibody has a CDR1 and a CDR3 having amino acid sequences of the CDR1 and the CDR3 as set forth in SEQ ID NO:44. In some embodiments, the single domain antibody has a CDR2 and a CDR3 having amino acid sequences of the CDR2 and the CDR3 as set forth in SEQ ID NO:44. In some embodiments, the single domain antibody has a CDR1, a CDR2, and a CDR3 having amino acid sequences of the CDR1, the CDR2, and the CDR3 as set forth in SEQ ID NO:44. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:45. In some embodiments, the single domain antibody has a CDR2 having an amino acid sequence of the CDR2 as set forth in SEQ ID NO:45. In other embodiments, the single domain antibody has a CDR3 having an amino acid sequence of the CDR3 as set forth in SEQ ID NO:45. In some embodiments, the single domain antibody has a CDR1 and a CDR2 having amino acid sequences of the CDR1 and the CDR2 as set forth in SEQ ID NO:45. In some embodiments, the single domain antibody has a CDR1 and a CDR3 having amino acid sequences of the CDR1 and the CDR3 as set forth in SEQ ID NO:45. In some embodiments, the single domain antibody has a CDR2 and a CDR3 having amino acid sequences of the CDR2 and the CDR3 as set forth in SEQ ID NO:45. In some embodiments, the single domain antibody has a CDR1, a CDR2, and a CDR3 having amino acid sequences of the CDR1, the CDR2, and the CDR3 as set forth in SEQ ID NO:45. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:46. In some embodiments, the single domain antibody has a CDR2 having an amino acid sequence of the CDR2 as set forth in SEQ ID NO:46. In other embodiments, the single domain antibody has a CDR3 having an amino acid sequence of the CDR3 as set forth in SEQ ID NO:46. In some embodiments, the single domain antibody has a CDR1 and a CDR2 having amino acid sequences of the CDR1 and the CDR2 as set forth in SEQ ID NO:46. In some embodiments, the single domain antibody has a CDR1 and a CDR3 having amino acid sequences of the CDR1 and the CDR3 as set forth in SEQ ID NO:46. In some embodiments, the single domain antibody has a CDR2 and a CDR3 having amino acid sequences of the CDR2 and the CDR3 as set forth in SEQ ID NO:46. In some embodiments, the single domain antibody has a CDR1, a CDR2, and a CDR3 having amino acid sequences of the CDR1, the CDR2, and the CDR3 as set forth in SEQ ID NO:46. CDR sequences can be determined according to well-known numbering systems. In some embodiments, the CDRs are according to IMGT numbering. In some embodiments, the CDRs are according to Kabat numbering. In some embodiments, the CDRs are according to AbM numbering In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:47. In some embodiments, the single domain antibody has a CDR2 having an amino acid sequence of the CDR2 as set forth in SEQ ID NO:47. In other embodiments, the single domain antibody has a CDR3 having an amino acid sequence of the CDR3 as set forth in SEQ ID NO:47. In some embodiments, the single domain antibody has a CDR1 and a CDR2 having amino acid sequences of the CDR1 and the CDR2 as set forth in SEQ ID NO:47. In some embodiments, the single domain antibody has a CDR1 and a CDR3 having amino acid sequences of the CDR1 and the CDR3 as set forth in SEQ ID NO:47. In some embodiments, the single domain antibody has a CDR2 and a CDR3 having amino acid sequences of the CDR2 and the CDR3 as set forth in SEQ ID NO:47. In some embodiments, the single domain antibody has a CDR1, a CDR2, and a CDR3 having amino acid sequences of the CDR1, the CDR2, and the CDR3 as set forth in SEQ ID NO:47. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:48. In some embodiments, the single domain antibody has a CDR2 having an amino acid sequence of the CDR2 as set forth in SEQ ID NO:48. In other embodiments, the single domain antibody has a CDR3 having an amino acid sequence of the CDR3 as set forth in SEQ ID NO:48. In some embodiments, the single domain antibody has a CDR1 and a CDR2 having amino acid sequences of the CDR1 and the CDR2 as set forth in SEQ ID NO:48. In some embodiments, the single domain antibody has a CDR1 and a CDR3 having amino acid sequences of the CDR1 and the CDR3 as set forth in SEQ ID NO:48. In some embodiments, the single domain antibody has a CDR2 and a CDR3 having amino acid sequences of the CDR2 and the CDR3 as set forth in SEQ ID NO:48. In some embodiments, the single domain antibody has a CDR1, a CDR2, and a CDR3 having amino acid sequences of the CDR1, the CDR2, and the CDR3 as set forth in SEQ ID NO:48. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:49. In some embodiments, the single domain antibody has a CDR2 having an amino acid sequence of the CDR2 as set forth in SEQ ID NO:49. In other embodiments, the single domain antibody has a CDR3 having an amino acid sequence of the CDR3 as set forth in SEQ ID NO:49. In some embodiments, the single domain antibody has a CDR1 and a CDR2 having amino acid sequences of the CDR1 and the CDR2 as set forth in SEQ ID NO:49. In some embodiments, the single domain antibody has a CDR1 and a CDR3 having amino acid sequences of the CDR1 and the CDR3 as set forth in SEQ ID NO:49. In some embodiments, the single domain antibody has a CDR2 and a CDR3 having amino acid sequences of the CDR2 and the CDR3 as set forth in SEQ ID NO:49. In some embodiments, the single domain antibody has a CDR1, a CDR2, and a CDR3 having amino acid sequences of the CDR1, the CDR2, and the CDR3 as set forth in SEQ ID NO:49. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:50. In some embodiments, the single domain antibody has a CDR2 having an amino acid sequence of the CDR2 as set forth in SEQ ID NO:50. In other embodiments, the single domain antibody has a CDR3 having an amino acid sequence of the CDR3 as set forth in SEQ ID NO:50. In some embodiments, the single domain antibody has a CDR1 and a CDR2 having amino acid sequences of the CDR1 and the CDR2 as set forth in SEQ ID NO:50. In some embodiments, the single domain antibody has a CDR1 and a CDR3 having amino acid sequences of the CDR1 and the CDR3 as set forth in SEQ ID NO:50. In some embodiments, the single domain antibody has a CDR2 and a CDR3 having amino acid sequences of the CDR2 and the CDR3 as set forth in SEQ ID NO:50. In some embodiments, the single domain antibody has a CDR1, a CDR2, and a CDR3 having amino acid sequences of the CDR1, the CDR2, and the CDR3 as set forth in SEQ ID NO:50. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:51. In some embodiments, the single domain antibody has a CDR2 having an amino acid sequence of the CDR2 as set forth in SEQ ID NO:51. In other embodiments, the single domain antibody has a CDR3 having an amino acid sequence of the CDR3 as set forth in SEQ ID NO:51. In some embodiments, the single domain antibody has a CDR1 and a CDR2 having amino acid sequences of the CDR1 and the CDR2 as set forth in SEQ ID NO:51. In some embodiments, the single domain antibody has a CDR1 and a CDR3 having amino acid sequences of the CDR1 and the CDR3 as set forth in SEQ ID NO:51. In some embodiments, the single domain antibody has a CDR2 and a CDR3 having amino acid sequences of the CDR2 and the CDR3 as set forth in SEQ ID NO:51. In some embodiments, the single domain antibody has a CDR1, a CDR2, and a CDR3 having amino acid sequences of the CDR1, the CDR2, and the CDR3 as set forth in SEQ ID NO:51. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:77. In some embodiments, the single domain antibody has a CDR2 having an amino acid sequence of the CDR2 as set forth in SEQ ID NO:77. In other embodiments, the single domain antibody has a CDR3 having an amino acid sequence of the CDR3 as set forth in SEQ ID NO:77. In some embodiments, the single domain antibody has a CDR1 and a CDR2 having amino acid sequences of the CDR1 and the CDR2 as set forth in SEQ ID NO:77. In some embodiments, the single domain antibody has a CDR1 and a CDR3 having amino acid sequences of the CDR1 and the CDR3 as set forth in SEQ ID NO:77. In some embodiments, the single domain antibody has a CDR2 and a CDR3 having amino acid sequences of the CDR2 and the CDR3 as set forth in SEQ ID NO:77. In some embodiments, the single domain antibody has a CDR1, a CDR2, and a CDR3 having amino acid sequences of the CDR1, the CDR2, and the CDR3 as set forth in SEQ ID NO:77. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:78. In some embodiments, the single domain antibody has a CDR2 having an amino acid sequence of the CDR2 as set forth in SEQ ID NO:78. In other embodiments, the single domain antibody has a CDR3 having an amino acid sequence of the CDR3 as set forth in SEQ ID NO:78. In some embodiments, the single domain antibody has a CDR1 and a CDR2 having amino acid sequences of the CDR1 and the CDR2 as set forth in SEQ ID NO:78. In some embodiments, the single domain antibody has a CDR1 and a CDR3 having amino acid sequences of the CDR1 and the CDR3 as set forth in SEQ ID NO:78. In some embodiments, the single domain antibody has a CDR2 and a CDR3 having amino acid sequences of the CDR2 and the CDR3 as set forth in SEQ ID NO:78. In some embodiments, the single domain antibody has a CDR1, a CDR2, and a CDR3 having amino acid sequences of the CDR1, the CDR2, and the CDR3 as set forth in SEQ ID NO:78. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:79. In some embodiments, the single domain antibody has a CDR2 having an amino acid sequence of the CDR2 as set forth in SEQ ID NO:79. In other embodiments, the single domain antibody has a CDR3 having an amino acid sequence of the CDR3 as set forth in SEQ ID NO:79. In some embodiments, the single domain antibody has a CDR1 and a CDR2 having amino acid sequences of the CDR1 and the CDR2 as set forth in SEQ ID NO:79. In some embodiments, the single domain antibody has a CDR1 and a CDR3 having amino acid sequences of the CDR1 and the CDR3 as set forth in SEQ ID NO:79. In some embodiments, the single domain antibody has a CDR2 and a CDR3 having amino acid sequences of the CDR2 and the CDR3 as set forth in SEQ ID NO:79. In some embodiments, the single domain antibody has a CDR1, a CDR2, and a CDR3 having amino acid sequences of the CDR1, the CDR2, and the CDR3 as set forth in SEQ ID NO:79. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:80. In some embodiments, the single domain antibody has a CDR2 having an amino acid sequence of the CDR2 as set forth in SEQ ID NO:80. In other embodiments, the single domain antibody has a CDR3 having an amino acid sequence of the CDR3 as set forth in SEQ ID NO:80. In some embodiments, the single domain antibody has a CDR1 and a CDR2 having amino acid sequences of the CDR1 and the CDR2 as set forth in SEQ ID NO:80. In some embodiments, the single domain antibody has a CDR1 and a CDR3 having amino acid sequences of the CDR1 and the CDR3 as set forth in SEQ ID NO:80. In some embodiments, the single domain antibody has a CDR2 and a CDR3 having amino acid sequences of the CDR2 and the CDR3 as set forth in SEQ ID NO:80. In some embodiments, the single domain antibody has a CDR1, a CDR2, and a CDR3 having amino acid sequences of the CDR1, the CDR2, and the CDR3 as set forth in SEQ ID NO:80. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:81. In some embodiments, the single domain antibody has a CDR2 having an amino acid sequence of the CDR2 as set forth in SEQ ID NO:81. In other embodiments, the single domain antibody has a CDR3 having an amino acid sequence of the CDR3 as set forth in SEQ ID NO:81. In some embodiments, the single domain antibody has a CDR1 and a CDR2 having amino acid sequences of the CDR1 and the CDR2 as set forth in SEQ ID NO:81. In some embodiments, the single domain antibody has a CDR1 and a CDR3 having amino acid sequences of the CDR1 and the CDR3 as set forth in SEQ ID NO:81. In some embodiments, the single domain antibody has a CDR2 and a CDR3 having amino acid sequences of the CDR2 and the CDR3 as set forth in SEQ ID NO:81. In some embodiments, the single domain antibody has a CDR1, a CDR2, and a CDR3 having amino acid sequences of the CDR1, the CDR2, and the CDR3 as set forth in SEQ ID NO:81. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:82. In some embodiments, the single domain antibody has a CDR2 having an amino acid sequence of the CDR2 as set forth in SEQ ID NO:82. In other embodiments, the single domain antibody has a CDR3 having an amino acid sequence of the CDR3 as set forth in SEQ ID NO:82. In some embodiments, the single domain antibody has a CDR1 and a CDR2 having amino acid sequences of the CDR1 and the CDR2 as set forth in SEQ ID NO:82. In some embodiments, the single domain antibody has a CDR1 and a CDR3 having amino acid sequences of the CDR1 and the CDR3 as set forth in SEQ ID NO:82. In some embodiments, the single domain antibody has a CDR2 and a CDR3 having amino acid sequences of the CDR2 and the CDR3 as set forth in SEQ ID NO:82. In some embodiments, the single domain antibody has a CDR1, a CDR2, and a CDR3 having amino acid sequences of the CDR1, the CDR2, and the CDR3 as set forth in SEQ ID NO:82. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:83. In some embodiments, the single domain antibody has a CDR2 having an amino acid sequence of the CDR2 as set forth in SEQ ID NO: 83. In other embodiments, the single domain antibody has a CDR3 having an amino acid sequence of the CDR3 as set forth in SEQ ID NO: 83. In some embodiments, the single domain antibody has a CDR1 and a CDR2 having amino acid sequences of the CDR1 and the CDR2 as set forth in SEQ ID NO: 83. In some embodiments, the single domain antibody has a CDR1 and a CDR3 having amino acid sequences of the CDR1 and the CDR3 as set forth in SEQ ID NO:83. In some embodiments, the single domain antibody has a CDR2 and a CDR3 having amino acid sequences of the CDR2 and the CDR3 as set forth in SEQ ID NO: 83. In some embodiments, the single domain antibody has a CDR1, a CDR2, and a CDR3 having amino acid sequences of the CDR1, the CDR2, and the CDR3 as set forth in SEQ ID NO:83. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:84. In some embodiments, the single domain antibody has a CDR2 having an amino acid sequence of the CDR2 as set forth in SEQ ID NO:84. In other embodiments, the single domain antibody has a CDR3 having an amino acid sequence of the CDR3 as set forth in SEQ ID NO:84. In some embodiments, the single domain antibody has a CDR1 and a CDR2 having amino acid sequences of the CDR1 and the CDR2 as set forth in SEQ ID NO:84. In some embodiments, the single domain antibody has a CDR1 and a CDR3 having amino acid sequences of the CDR1 and the CDR3 as set forth in SEQ ID NO:84. In some embodiments, the single domain antibody has a CDR2 and a CDR3 having amino acid sequences of the CDR2 and the CDR3 as set forth in SEQ ID NO:84. In some embodiments, the single domain antibody has a CDR1, a CDR2, and a CDR3 having amino acid sequences of the CDR1, the CDR2, and the CDR3 as set forth in SEQ ID NO:84. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO:85. In some embodiments, the single domain antibody has a CDR2 having an amino acid sequence of the CDR2 as set forth in SEQ ID NO:85. In other embodiments, the single domain antibody has a CDR3 having an amino acid sequence of the CDR3 as set forth in SEQ ID NO:S5. In some embodiments, the single domain antibody has a CDR1 and a CDR2 having amino acid sequences of the CDR1 and the CDR2 as set forth in SEQ ID NO:S5. In some embodiments, the single domain antibody has a CDR1 and a CDR3 having amino acid sequences of the CDR1 and the CDR3 as set forth in SEQ ID NO:S5. In some embodiments, the single domain antibody has a CDR2 and a CDR3 having amino acid sequences of the CDR2 and the CDR3 as set forth in SEQ ID NO:85. In some embodiments, the single domain antibody has a CDR1, a CDR2, and a CDR3 having amino acid sequences of the CDR1, the CDR2, and the CDR3 as set forth in SEQ ID NO:85. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In others embodiments, the CDRs are according to a combination of various numbering systems, e.g., a combination of Kabat and Chothia numbering systems, or a combination of Kabat and IMGT numbering systems. In some embodiments, the anti-Claudin18.2 single domain antibody is camelid. In some embodiments, the anti-Claudin18.2 single domain antibody is humanized. In some embodiments, the anti-Claudin18.2 single domain antibody comprises an acceptor human framework, e.g., a human immunoglobulin framework or a human consensus framework.

In some embodiments, the anti-Claudin18.2 sdAb comprises a CDR1 comprising the amino acid sequence of SEQ ID NO:1 or SEQ ID NO:113; a CDR2 comprising the amino acid sequence of SEQ ID NO:12; and a CDR3 comprising the amino acid sequence of SEQ ID NO:24 or SEQ ID NO:126. In some embodiments, the anti-Claudin18.2 sdAb comprises a CDR1 comprising the amino acid sequence of SEQ ID NO:1; a CDR2 comprising the amino acid sequence of SEQ ID NO:12; and a CDR3 comprising the amino acid sequence of SEQ ID NO:24. In some embodiments, the anti-Claudin18.2 sdAb comprises a CDR1 comprising the amino acid sequence of SEQ ID NO:113; a CDR2 comprising the amino acid sequence of SEQ ID NO:12; and a CDR3 comprising the amino acid sequence of SEQ ID NO:126.

In some embodiments, the anti-Claudin18.2 sdAb comprises a CDR1 comprising the amino acid sequence of SEQ ID NO:2 or SEQ ID NO:114; a CDR2 comprising the amino acid sequence of SEQ ID NO:13; and a CDR3 comprising the amino acid sequence of SEQ ID NO:25 or SEQ ID NO:127. In some embodiments, the anti-Claudin18.2 sdAb comprises a CDR1 comprising the amino acid sequence of SEQ ID NO:2; a CDR2 comprising the amino acid sequence of SEQ ID NO:13; and a CDR3 comprising the amino acid sequence of SEQ ID NO:25. In some embodiments, the anti-Claudin18.2 sdAb comprises a CDR1 comprising the amino acid sequence of SEQ ID NO:114; a CDR2 comprising the amino acid sequence of SEQ ID NO:13; and a CDR3 comprising the amino acid sequence of SEQ ID NO:127.

In some embodiments, the anti-Claudin18.2 sdAb comprises a CDR1 comprising the amino acid sequence of SEQ ID NO:3 or SEQ ID NO:115; a CDR2 comprising the amino acid sequence of SEQ ID NO:14; and a CDR3 comprising the amino acid sequence of SEQ ID NO:26 or SEQ ID NO:128. In some embodiments, the anti-Claudin18.2 sdAb comprises a CDR1 comprising the amino acid sequence of SEQ ID NO:3; a CDR2 comprising the amino acid sequence of SEQ ID NO:14; and a CDR3 comprising the amino acid sequence of SEQ ID NO:26. In some embodiments, the anti-Claudin18.2 sdAb comprises a CDR1 comprising the amino acid sequence of SEQ ID NO:115; a CDR2 comprising the amino acid sequence of SEQ ID NO: 14; and a CDR3 comprising the amino acid sequence of SEQ ID NO:128.

In some embodiments, the anti-Claudin18.2 sdAb comprises a CDR1 comprising the amino acid sequence of SEQ ID NO:4 or SEQ ID NO: 116; a CDR2 comprising the amino acid sequence of SEQ ID NO: 15; and a CDR3 comprising the amino acid sequence of SEQ ID NO:27 or SEQ ID NO:129. In some embodiments, the anti-Claudin18.2 sdAb comprises a CDR1 comprising the amino acid sequence of SEQ ID NO:4; a CDR2 comprising the amino acid sequence of SEQ ID NO: 15; and a CDR3 comprising the amino acid sequence of SEQ ID NO:27. In some embodiments, the anti-Claudin18.2

sdAb comprises a CDR1 comprising the amino acid sequence of SEQ ID NO:116; a CDR2 comprising the amino acid sequence of SEQ ID NO: 15; and a CDR3 comprising the amino acid sequence of SEQ ID NO:129.

In some embodiments, the anti-Claudin18.2 sdAb comprises a CDR1 comprising the amino acid sequence of SEQ ID NO:5 or SEQ ID NO:117; a CDR2 comprising the amino acid sequence of SEQ ID NO: 16; and a CDR3 comprising the amino acid sequence of SEQ ID NO:28 or SEQ ID NO:130. In some embodiments, the anti-Claudin18.2 sdAb comprises a CDR1 comprising the amino acid sequence of SEQ ID NO:5; a CDR2 comprising the amino acid sequence of SEQ ID NO: 16; and a CDR3 comprising the amino acid sequence of SEQ ID NO:28. In some embodiments, the anti-Claudin18.2 sdAb comprises a CDR1 comprising the amino acid sequence of SEQ ID NO:117; a CDR2 comprising the amino acid sequence of SEQ ID NO: 16; and a CDR3 comprising the amino acid sequence of SEQ ID NO:130.

In some embodiments, the anti-Claudin18.2 sdAb comprises a CDR1 comprising the amino acid sequence of SEQ ID NO:6 or SEQ ID NO: 118; a CDR2 comprising the amino acid sequence of SEQ ID NO: 17; and a CDR3 comprising the amino acid sequence of SEQ ID NO:29 or SEQ ID NO:131. In some embodiments, the anti-Claudin18.2 sdAb comprises a CDR1 comprising the amino acid sequence of SEQ ID NO:6; a CDR2 comprising the amino acid sequence of SEQ ID NO: 17; and a CDR3 comprising the amino acid sequence of SEQ ID NO 29. In some embodiments, the anti-Claudin18.2 sdAb comprises a CDR1 comprising the amino acid sequence of SEQ ID NO:118; a CDR2 comprising the amino acid sequence of SEQ ID NO: 17; and a CDR3 comprising the amino acid sequence of SEQ ID NO:131.

In some embodiments, the anti-Claudin18.2 sdAb comprises a CDR1 comprising the amino acid sequence of SEQ ID NO:7 or SEQ ID NO: 119; a CDR2 comprising the amino acid sequence of SEQ ID NO: 18; and a CDR3 comprising the amino acid sequence of SEQ ID NO:30 or SEQ ID NO:132. In some embodiments, the anti-Claudin18.2 sdAb comprises a CDR1 comprising the amino acid sequence of SEQ ID NO:7; a CDR2 comprising the amino acid sequence of SEQ ID NO: 18; and a CDR3 comprising the amino acid sequence of SEQ ID NO:30. In some embodiments, the anti-Claudin18.2 sdAb comprises a CDR1 comprising the amino acid sequence of SEQ ID NO:119; a CDR2 comprising the amino acid sequence of SEQ ID NO: 18; and a CDR3 comprising the amino acid sequence of SEQ ID NO:132.

In some embodiments, the anti-Claudin18.2 sdAb comprises a CDR1 comprising the amino acid sequence of SEQ ID NO:8 or SEQ ID NO: 120; a CDR2 comprising the amino acid sequence of SEQ ID NO: 19; and a CDR3 comprising the amino acid sequence of SEQ ID NO:31 or SEQ ID NO:133. In some embodiments, the anti-Claudin18.2 sdAb comprises a CDR1 comprising the amino acid sequence of SEQ ID NO:8; a CDR2 comprising the amino acid sequence of SEQ ID NO: 19; and a CDR3 comprising the amino acid sequence of SEQ ID NO:31. In some embodiments, the anti-Claudin18.2 sdAb comprises a CDR1 comprising the amino acid sequence of SEQ ID NO:120; a CDR2 comprising the amino acid sequence of SEQ ID NO:19; and a CDR3 comprising the amino acid sequence of SEQ ID NO:133.

In some embodiments, the anti-Claudin18.2 sdAb comprises a CDR1 comprising the amino acid sequence of SEQ ID NO:1 or SEQ ID NO:121; a CDR2 comprising the amino acid sequence of SEQ ID NO:20; and a CDR3 comprising the amino acid sequence of SEQ ID NO:32 or SEQ ID NO:134. In some embodiments, the anti-Claudin18.2 sdAb comprises a CDR1 comprising the amino acid sequence of SEQ ID NO:1; a CDR2 comprising the amino acid sequence of SEQ ID NO:20; and a CDR3 comprising the amino acid sequence of SEQ ID NO:32. In some embodiments, the anti-Claudin18.2 sdAb comprises a CDR1 comprising the amino acid sequence of SEQ ID NO:121; a CDR2 comprising the amino acid sequence of SEQ ID NO:20; and a CDR3 comprising the amino acid sequence of SEQ ID NO:134.

In some embodiments, the anti-Claudin18.2 sdAb comprises a CDR1 comprising the amino acid sequence of SEQ ID NO:5 or SEQ ID NO: 122; a CDR2 comprising the amino acid sequence of SEQ ID NO:21; and a CDR3 comprising the amino acid sequence of SEQ ID NO:33 or SEQ ID NO: 135. In some embodiments, the anti-Claudin18.2 sdAb comprises a CDR1 comprising the amino acid sequence of SEQ ID NO:5; a CDR2 comprising the amino acid sequence of SEQ ID NO:21; and a CDR3 comprising the amino acid sequence of SEQ ID NO:33. In some embodiments, the anti-Claudin18.2 sdAb comprises a CDR1 comprising the amino acid sequence of SEQ ID NO:122; a CDR2 comprising the amino acid sequence of SEQ ID NO:21; and a CDR3 comprising the amino acid sequence of SEQ ID NO:135.

In some embodiments, the anti-Claudin18.2 sdAb comprises a CDR1 comprising the amino acid sequence of SEQ ID NO:9 or SEQ ID NO: 123; a CDR2 comprising the amino acid sequence of SEQ ID NO:22; and a CDR3 comprising the amino acid sequence of SEQ ID NO:34 or SEQ ID NO:136. In some embodiments, the anti-Claudin18.2 sdAb comprises a CDR1 comprising the amino acid sequence of SEQ ID NO:9; a CDR2 comprising the amino acid sequence of SEQ ID NO:22; and a CDR3 comprising the amino acid sequence of SEQ ID NO:34. In some embodiments, the anti-Claudin18.2 sdAb comprises a CDR1 comprising the amino acid sequence of SEQ ID NO:123; a CDR2 comprising the amino acid sequence of SEQ ID NO:22; and a CDR3 comprising the amino acid sequence of SEQ ID NO:136.

In some embodiments, the anti-Claudin18.2 sdAb comprises a CDR1 comprising the amino acid sequence of SEQ ID NO:10 or SEQ ID NO:124; a CDR2 comprising the amino acid sequence of SEQ ID NO:23; and a CDR3 comprising the amino acid sequence of SEQ ID NO:35 or SEQ ID NO:137. In some embodiments, the anti-Claudin18.2 sdAb comprises a CDR1 comprising the amino acid sequence of SEQ ID NO:10; a CDR2 comprising the amino acid sequence of SEQ ID NO:23; and a CDR3 comprising the amino acid sequence of SEQ ID NO:35. In some embodiments, the anti-Claudin18.2 sdAb comprises a CDR1 comprising the amino acid sequence of SEQ ID NO:124; a CDR2 comprising the amino acid sequence of SEQ ID NO:23; and a CDR3 comprising the amino acid sequence of SEQ ID NO:137.

In some embodiments, the anti-Claudin18.2 sdAb comprises a CDR1 comprising the amino acid sequence of SEQ ID NO:5 or SEQ ID NO: 122; a CDR2 comprising the amino acid sequence of SEQ ID NO:21; and a CDR3 comprising the amino acid sequence of SEQ ID NO:36 or SEQ ID NO:138. In some embodiments, the anti-Claudin18.2 sdAb comprises a CDR1 comprising the amino acid sequence of SEQ ID NO:5; a CDR2 comprising the amino acid sequence of SEQ ID NO:21; and a CDR3 comprising the amino acid sequence of SEQ ID NO:36. In some embodiments, the anti-Claudin18.2 sdAb comprises a CDR1 comprising the amino acid sequence of SEQ ID NO:122; a CDR2 comprising the amino acid sequence of SEQ ID NO:21; and a CDR3 comprising the amino acid sequence of SEQ ID NO:138.

In some embodiments, the anti-Claudin18.2 sdAb comprises a CDR1 comprising the amino acid sequence of SEQ ID NO:11 or SEQ ID NO:125; a CDR2 comprising the amino acid sequence of SEQ ID NO: 13; and a CDR3 comprising the amino acid sequence of SEQ ID NO:37 or SEQ ID NO:139. In some embodiments, the anti-Claudin18.2 sdAb comprises a CDR1 comprising the amino acid sequence of SEQ ID NO:11; a CDR2 comprising the amino acid sequence of SEQ ID NO: 13; and a CDR3 comprising the amino acid sequence of SEQ ID NO:37. In some embodiments, the anti-Claudin18.2 sdAb comprises a CDR1 comprising the amino acid sequence of SEQ ID NO:125; a CDR2 comprising the amino acid sequence of SEQ ID NO: 13; and a CDR3 comprising the amino acid sequence of SEQ ID NO:139.

In some embodiments, the single domain antibody further comprises one or more framework region(s) of the single domain antibodies in Table 2. In some embodiments, the single domain antibody comprises one or more framework(s) derived from a VHH domain comprising the sequence of SEQ ID NO:38. In some embodiments, the single domain antibody comprises one or more framework(s) derived from a VHH domain comprising the sequence of SEQ ID NO:39. In some embodiments, the single domain antibody comprises one or more framework(s) derived from a VHH domain comprising the sequence of SEQ ID NO:40. In some embodiments, the single domain antibody comprises one or more framework(s) derived from a VHH domain comprising the sequence of SEQ ID NO:41. In some embodiments, the single domain antibody comprises one or more framework(s) derived from a VHH domain comprising the sequence of SEQ ID NO:42. In some embodiments, the single domain antibody comprises one or more framework(s) derived from a VHH domain comprising the sequence of SEQ ID NO:43. In some embodiments, the single domain antibody comprises one or more framework(s) derived from a VHH domain comprising the sequence of SEQ ID NO:44. In some embodiments, the single domain antibody comprises one or more framework(s) derived from a VHH domain comprising the sequence of SEQ ID NO:45. In some embodiments, the single domain antibody comprises one or more framework(s) derived from a VHH domain comprising the sequence of SEQ ID NO:46. In some embodiments, the single domain antibody comprises one or more framework(s) derived from a VHH domain comprising the sequence of SEQ ID NO:47. In some embodiments, the single domain antibody comprises one or more framework(s) derived from a VHH domain comprising the sequence of SEQ ID NO:48. In some embodiments, the single domain antibody comprises one or more framework(s) derived from a VHH domain comprising the sequence of SEQ ID NO:49. In some embodiments, the single domain antibody comprises one or more framework(s) derived from a VHH domain comprising the sequence of SEQ ID NO:50. In some embodiments, the single domain antibody comprises one or more framework(s) derived from a VHH domain comprising the sequence of SEQ ID NO:51. In some embodiments, the single domain antibody comprises one or more framework(s) derived from a VHH domain comprising the sequence of SEQ ID NO:77. In some embodiments, the single domain antibody comprises one or more framework(s) derived from a VHH domain comprising the sequence of SEQ ID NO:78. In some embodiments, the single domain antibody comprises one or more framework(s) derived from a VHH domain comprising the sequence of SEQ ID NO:79. In some embodiments, the single domain antibody comprises one or more framework(s) derived from a VHH domain comprising the sequence of SEQ ID NO:80. In some embodiments, the single domain antibody comprises one or more framework(s) derived from a VHH domain comprising the sequence of SEQ ID NO:81. In some embodiments, the single domain antibody comprises one or more framework(s) derived from a VHH domain comprising the sequence of SEQ ID NO:82. In some embodiments, the single domain antibody comprises one or more framework(s) derived from a VHH domain comprising the sequence of SEQ ID NO:83. In some embodiments, the single domain antibody comprises one or more framework(s) derived from a VHH domain comprising the sequence of SEQ ID NO:84. In some embodiments, the single domain antibody comprises one or more framework(s) derived from a VHH domain comprising the sequence of SEQ ID NO:85.

In some embodiments, the single domain antibody provided herein is a humanized single domain antibody. In some embodiments, humanized single domain antibodies can be generated using the method exemplified in the Section 6 below or the methods described in the section below.

Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, there is provided an isolated anti-Claudin18.2 single domain antibody comprising a VHH domain having the amino acid sequence of SEQ ID NO:38. In some embodiments, there is provided a polypeptide comprising the amino acid sequence of SEQ ID NO:38. In some embodiments, there is provided an isolated anti-Claudin18.2 single domain antibody comprising a VHH domain having the amino acid sequence of SEQ ID NO:39. In some embodiments, there is provided a polypeptide comprising the amino acid sequence of SEQ ID NO:39. In some embodiments, there is provided an isolated anti-Claudin18.2 single domain antibody comprising a VHH domain having the amino acid sequence of SEQ ID NO:40. In some embodiments, there is provided a polypeptide comprising the amino acid sequence of SEQ ID NO:40. In some embodiments, there is provided an isolated anti-Claudin18.2 single domain antibody comprising a VHH domain having the amino acid sequence of SEQ ID NO:41. In some embodiments, there is provided a polypeptide comprising the amino acid sequence of SEQ ID NO:41. In some embodiments, there is provided an isolated anti-Claudin18.2 single domain antibody comprising a VHH domain having the amino acid sequence of SEQ ID NO:42. In some embodiments, there is provided a polypeptide comprising the amino acid sequence of SEQ ID NO:42. In some embodiments, there is provided an isolated anti-Claudin18.2 single domain antibody comprising a VHH domain having the amino acid sequence of SEQ ID NO:43. In some embodiments, there is provided a polypeptide comprising the amino acid sequence of SEQ ID NO:43. In some embodiments, there is provided an isolated anti-Claudin18.2 single domain antibody comprising a VHH domain having the amino acid sequence of SEQ ID NO:44. In some embodiments, there is provided a polypeptide comprising the amino acid sequence of SEQ ID NO:44. In some embodiments, there is provided an isolated anti-Claudin18.2 single domain antibody comprising a VHH domain having the amino acid sequence of SEQ ID NO:45. In some embodiments, there is provided a polypeptide comprising the amino acid sequence of SEQ ID NO:45. In some embodiments, there is provided an isolated anti-Claudin18.2 single domain antibody comprising a VHH domain having the amino acid sequence of SEQ ID NO:46. In some embodiments, there is provided a polypeptide comprising the amino acid sequence of SEQ ID NO:46. In some embodiments, there is provided an isolated anti-Claudin18.2 single domain antibody comprising a VHH domain having the amino acid sequence of SEQ ID NO:47. In some embodiments, there is provided a polypeptide comprising the amino acid sequence of SEQ ID NO:47. In some embodiments, there is provided an isolated anti-Claudin18.2 single domain antibody comprising a VHH domain having the amino acid sequence of SEQ ID NO:48. In some embodiments, there is provided a polypeptide comprising the amino acid sequence of SEQ ID NO:48. In some embodiments, there is provided an isolated anti-Claudin18.2 single domain antibody comprising a VHH domain having the amino acid sequence of SEQ ID NO:49. In some embodiments, there is provided a polypeptide comprising the amino acid sequence of SEQ ID NO:49. In some embodiments, there is provided an isolated anti-Claudin18.2 single domain antibody comprising a VHH domain having the amino acid sequence of SEQ ID NO:50. In some embodiments, there is provided a polypeptide comprising the amino acid sequence of SEQ ID NO:50. In some embodiments, there is provided an isolated anti-Claudin18.2 single domain antibody comprising a VHH domain having the amino acid sequence of SEQ ID NO:51. In some embodiments, there is provided a polypeptide comprising the amino acid sequence of SEQ ID NO:51. In some embodiments, there is provided an isolated anti-Claudin18.2 single domain antibody comprising a VHH domain having the amino acid sequence of SEQ ID NO:77. In some embodiments, there is provided a polypeptide comprising the amino acid sequence of SEQ ID NO:77. In some embodiments, there is provided an isolated anti-Claudin18.2 single domain antibody comprising a VHH domain having the amino acid sequence of SEQ ID NO:78. In some embodiments, there is provided a polypeptide comprising the amino acid sequence of SEQ ID NO:78. In some embodiments, there is provided an isolated anti-Claudin18.2 single domain antibody comprising a VHH domain having the amino acid sequence of SEQ ID NO:79. In some embodiments, there is provided a polypeptide comprising the amino acid sequence of SEQ ID NO:79. In some embodiments, there is provided an isolated anti-Claudin18.2 single domain antibody comprising a VHH domain having the amino acid sequence of SEQ ID NO: 80. In some embodiments, there is provided a polypeptide comprising the amino acid sequence of SEQ ID NO: 80. In some embodiments, there is provided an isolated anti-Claudin18.2 single domain antibody comprising a VHH domain having the amino acid sequence of SEQ ID NO: 81. In some embodiments, there is provided a polypeptide comprising the amino acid sequence of SEQ ID NO: 81. In some embodiments, there is provided an isolated anti-Claudin18.2 single domain antibody comprising a VHH domain having the amino acid sequence of SEQ ID NO:82. In some embodiments, there is provided a polypeptide comprising the amino acid sequence of SEQ ID NO: 82. In some embodiments, there is provided an isolated anti-Claudin18.2 single domain antibody comprising a VHH domain having the amino acid sequence of SEQ ID NO: 83. In some embodiments, there is provided a polypeptide comprising the amino acid sequence of SEQ ID NO: 83. In some embodiments, there is provided an isolated anti-Claudin18.2 single domain antibody comprising a VHH domain having the amino acid sequence of SEQ ID NO: 84. In some embodiments, there is provided a polypeptide comprising the amino acid sequence of SEQ ID NO: 84. In some embodiments, there is provided an isolated anti-CIaudin18.2 single domain antibody comprising a VHH domain having the amino acid sequence of SEQ ID NO: 85. In some embodiments, there is provided a polypeptide comprising the amino acid sequence of SEQ ID NO:85.

In certain embodiments, an antibody described herein or an antigen-binding fragment thereof comprises amino acid sequences with certain percent identity relative to any one of antibodies described above including those in Table 2.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4: 11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, there is provided an anti-Claudin18.2 sdAb comprising three CDRs comprising: (a) a CDR1 having at least about 85%, 86%, 87%, 88%, 89%, 90%, 91%, 92%, 93%, 94%, 95%, 96%, 97%, 98%, 99%, or 100% sequence identity to an amino acid sequence selected from SEQ ID NOs:1-11 and 1 13-125; (b) a CDR2 having at least about 85%, 86%, 87%, 88%, 89%, 90%, 91%, 92%, 93%, 94%, 95%, 96%, 97%, 98%, 99%, or 100% sequence identity to an amino acid sequence selected from SEQ ID NOs: 12-23; and (c) a CDR3 having at least about 85%, 86%, 87%, 88%, 89%, 90%, 91%, 92%, 93%, 94%, 95%, 96%, 97%, 98%, 99%, or 100% sequence identity to an amino acid sequence selected from SEQ ID NOs:24-37 and 126-139. In some embodiments, a CDR having at least about any one of 85%, 86%, 87%, 88%, 89%, 90%, 91%, 92%, 93%, 94%, 95%, 96%, 97%, 98%, or 99% identity contains substitutions (e.g., conservative substitutions), insertions, or deletions relative to the reference sequence, but the anti-Claudin18.2 sdAb comprising that sequence retains the ability to bind to Claudin18.2. In some embodiments, there is provided an anti-Claudin18.2 sdAb comprising three CDRs comprising: (a) a CDR1 having about any one of 1, 2, 3, 4 or 5 amino acid substitutions (e.g., conservative substitutions), insertions, or deletions to an amino acid sequence selected from SEQ ID NOs: 1-11 and 113-125; (b) a CDR2 having about any one of 1, 2, 3, 4 or 5 amino acid substitutions (e.g., conservative substitutions), insertions, or deletions to an amino acid sequence selected from SEQ ID NOs: 12-23; and (c) a CDR3 having about any one of 1, 2, 3, 4 or 5 amino acid substitutions (e.g., conservative substitutions), insertions, or deletions to an amino acid sequence selected from SEQ ID NOs: 24-37 and 126-139. In some embodiments, the anti-Claudin18.2 sdAb is affinity matured. In some embodiments, the anti-Claudin18.2 sdAb is camelid. In some embodiments, the anti-Claudin18.2 sdAb is humanized. In some embodiments, the anti-Claudin18.2 sdAb comprises an acceptor human framework, e.g., a human immunoglobulin framework or a human consensus framework.

In some embodiments, a Claudin18.2 binding moiety comprises a sdAb comprising a variable domain (such as VHH domain) comprising (1) a CDR1 comprising the amino acid sequence selected from the group consisting of SEQ ID NOs: 1-11 and 113-125 or a variant thereof comprising 1, 2, 3, 4 or 5 amino acid substitutions, insertions, or deletions; (2) a CDR2 comprising the amino acid sequence selected from the group consisting of SEQ ID NOs: 12-23, or a variant thereof comprising 1, 2, 3, 4 or 5 amino acid substitutions, insertions, or deletions; and/or (3) a CDR3 comprising the amino acid sequence selected from the group consisting of SEQ ID NOs: 24-37 and 126-139, or a variant thereof comprising 1, 2, 3, 4 or 5 amino acid substitutions, insertions, or deletions. In some embodiments, the CDR (VHH CDR1, VHH CDR2, and/or VHH CDR3) comprises one amino acid substitution, insertions, or deletions. In some embodiments, the CDR (VHH CDR1, VHH CDR2, and/or VHH CDR3) comprises two amino acid substitutions, insertions, or deletions. In some embodiments, the CDR (VHH CDR1, VHH CDR2, and/or VHH CDR3) comprises three amino acid substitutions, insertions, or deletions. In some embodiments, the CDR (VHH CDR1, VHH CDR2, and/or VHH CDR3) comprises four amino acid substitutions, insertions, or deletions. In some embodiments, the CDR (VHH CDR1, VHH CDR2, and/or VHH CDR3) comprises five amino acid substitutions, insertions, or deletions. In some embodiments, the one or more amino acid substitutions are conservative substitutions. In some embodiments, the one or more substitutions are made for antibody humanization. In some embodiments, the one or more substitutions, insertions, or deletions are made for affinity maturation. In some embodiments, the one or more substitutions, insertions, or deletions are made for antibody optimization.

In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 38, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:39, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:40, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:41, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:42, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:43, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:44, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:45, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:46, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:47, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:48, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:49, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:50, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:51, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:77, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:78, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:79, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:80, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:81, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:82, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:83, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:84, wherein the single domain antibody binds to Claudin18.2. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:85, wherein the single domain antibody binds to Claudin18.2. In some embodiments, a VHH sequence having at least about any one of 75%, 80%, 85%, 86%, 87%, 88%, 89%, 90%, 91%, 92%, 93%, 94%, 95%, 96%, 97%, 98%, or 99% identity contains substitutions (e.g., conservative substitutions), insertions, or deletions relative to the reference sequence, but the anti-Claudin18.2 single domain antibody comprising that sequence retains the ability to bind to Claudin18.2. In some embodiments, a total of 1 to 10 amino acids have been substituted, inserted and/or deleted in an amino acid sequence selected from SEQ ID NOs:38-51 and 77-85. In some embodiments, substitutions, insertions, or deletions occur in regions outside the CDRs (i.e., in the FRs).

In some embodiments, functional epitopes can be mapped, e.g., by combinatorial alanine scanning, to identify amino acids in the Claudin18.2 protein that are necessary for interaction with anti-Claudin18.2 single domain antibodies provided herein. In some embodiments, conformational and crystal structure of anti-Claudin18.2 single domain antibody bound to Claudin18.2 may be employed to identify the epitopes. In some embodiments, the present disclosure provides an antibody that specifically binds to the same epitope as any of the anti-Claudin18.2 single domain antibodies provided herein. For example, in some embodiments, an antibody is provided that binds to the same epitope as an anti-Claudin18.2 single domain antibody comprising the amino acid sequence of SEQ ID NO:38. In some embodiments, an antibody is provided that binds to the same epitope as an anti-Claudin18.2 single domain antibody comprising the amino acid sequence of SEQ ID NO:39. In some embodiments, an antibody is provided that binds to the same epitope as an anti-Claudin18.2 single domain antibody comprising the amino acid sequence of SEQ ID NO:40. In some embodiments, an antibody is provided that binds to the same epitope as an anti-Claudin18.2 single domain antibody comprising the amino acid sequence of SEQ ID NO:41. In some embodiments, an antibody is provided that binds to the same epitope as an anti-Claudin18.2 single domain antibody comprising the amino acid sequence of SEQ ID NO:42. In some embodiments, an antibody is provided that binds to the same epitope as an anti-Claudin18.2 single domain antibody comprising the amino acid sequence of SEQ ID NO:43. In some embodiments, an antibody is provided that binds to the same epitope as an anti-Claudin18.2 single domain antibody comprising the amino acid sequence of SEQ ID NO:44. In some embodiments, an antibody is provided that binds to the same epitope as an anti-Claudin18.2 single domain antibody comprising the amino acid sequence of SEQ ID NO:45. In some embodiments, an antibody is provided that binds to the same epitope as an anti-Claudin18.2 single domain antibody comprising the amino acid sequence of SEQ ID NO:46. In some embodiments, an antibody is provided that binds to the same epitope as an anti-Claudin18.2 single domain antibody comprising the amino acid sequence of SEQ ID NO:47. In some embodiments, an antibody is provided that binds to the same epitope as an anti-Claudin18.2 single domain antibody comprising the amino acid sequence of SEQ ID NO:48. In some embodiments, an antibody is provided that binds to the same epitope as an anti-Claudin18.2 single domain antibody comprising the amino acid sequence of SEQ ID NO:49. In some embodiments, an antibody is provided that binds to the same epitope as an anti-Claudin18.2 single domain antibody comprising the amino acid sequence of SEQ ID NO:50. In some embodiments, an antibody is provided that binds to the same epitope as an anti-Claudin18.2 single domain antibody comprising the amino acid sequence of SEQ ID NO:51. In some embodiments, an antibody is provided that binds to the same epitope as an anti-Claudin18.2 single domain antibody comprising the amino acid sequence of SEQ ID NO:77. In some embodiments, an antibody is provided that binds to the same epitope as an anti-Claudin18.2 single domain antibody comprising the amino acid sequence of SEQ ID NO:78. In some embodiments, an antibody is provided that binds to the same epitope as an anti-Claudin18.2 single domain antibody comprising the amino acid sequence of SEQ ID NO:79. In some embodiments, an antibody is provided that binds to the same epitope as an anti-Claudin18.2 single domain antibody comprising the amino acid sequence of SEQ ID NO:80. In some embodiments, an antibody is provided that binds to the same epitope as an anti-Claudin18.2 single domain antibody comprising the amino acid sequence of SEQ ID NO: 81. In some embodiments, an antibody is provided that binds to the same epitope as an anti-Claudin18.2 single domain antibody comprising the amino acid sequence of SEQ ID NO:82. In some embodiments, an antibody is provided that binds to the same epitope as an anti-Claudin18.2 single domain antibody comprising the amino acid sequence of SEQ ID NO:83. In some embodiments, an antibody is provided that binds to the same epitope as an anti-Claudin18.2 single domain antibody comprising the amino acid sequence of SEQ ID NO: 84. In some embodiments, an antibody is provided that binds to the same epitope as an anti-Claudiny single domain antibody comprising the amino acid sequence of SEQ ID NO:85.

In some embodiments, provided herein is an anti-Claudin18.2 antibody, or antigen binding fragment thereof, that specifically binds to Claudin18.2 competitively with any one of the anti-Claudin18.2 single domain antibodies described herein. In some embodiments, competitive binding may be determined using an ELISA assay. For example, in some embodiments, an antibody is provided that specifically binds to Claudin18.2 competitively with an anti-Claudin18.2 single domain antibody comprising the amino acid sequence of SEQ ID NO:38. In some embodiments, an antibody is provided that specifically binds to Claudin18.2 competitively with an anti-Claudin18.2 single domain antibody comprising the amino acid sequence of SEQ ID NO:39. In some embodiments, an antibody is provided that specifically binds to Claudin18.2 competitively with an anti-Claudin18.2 single domain antibody comprising the amino acid sequence of SEQ ID NO:40. In some embodiments, an antibody is provided that specifically binds to Claudin18.2 competitively with an anti-Claudin18.2 single domain antibody comprising the amino acid sequence of SEQ ID NO:41. In some embodiments, an antibody is provided that specifically binds to Claudin18.2 competitively with an anti-Claudin18.2 single domain antibody comprising the amino acid sequence of SEQ ID NO:42. In some embodiments, an antibody is provided that specifically binds to Claudin18.2 competitively with an anti-Claudin18.2 single domain antibody comprising the amino acid sequence of SEQ ID NO:43. In some embodiments, an antibody is provided that specifically binds to Claudin18.2 competitively with an anti-Claudin18.2 single domain antibody comprising the amino acid sequence of SEQ ID NO:44. In some embodiments, an antibody is provided that specifically binds to Claudin18.2 competitively with an anti-Claudin18.2 single domain antibody comprising the amino acid sequence of SEQ ID NO:45. In some embodiments, an antibody is provided that specifically binds to Claudin18.2 competitively with an anti-Claudin18.2 single domain antibody comprising the amino acid sequence of SEQ ID NO:46. In some embodiments, an antibody is provided that specifically binds to Claudin18.2 competitively with an anti-Claudin18.2 single domain antibody comprising the amino acid sequence of SEQ ID NO:47. In some embodiments, an antibody is provided that specifically binds to Claudin18.2 competitively with an anti-Claudin18.2 single domain antibody comprising the amino acid sequence of SEQ ID NO:48. In some embodiments, an antibody is provided that specifically binds to Claudin18.2 competitively with an anti-Claudin18.2 single domain antibody comprising the amino acid sequence of SEQ ID NO:49. In some embodiments, an antibody is provided that specifically binds to Claudin18.2 competitively with an anti-Claudin18.2 single domain antibody comprising the amino acid sequence of SEQ ID NO:50. In some embodiments, an antibody is provided that specifically binds to Claudin18.2 competitively with an anti-Claudin18.2 single domain antibody comprising the amino acid sequence of SEQ ID NO:51. In some embodiments, an antibody is provided that specifically binds to Claudin18.2 competitively with an anti-Claudin18.2 single domain antibody comprising the amino acid sequence of SEQ ID NO:77. In some embodiments, an antibody is provided that specifically binds to Claudin18.2 competitively with an anti-Claudin18.2 single domain antibody comprising the amino acid sequence of SEQ ID NO:78. In some embodiments, an antibody is provided that specifically binds to Claudin18.2 competitively with an anti-Claudin18.2 single domain antibody comprising the amino acid sequence of SEQ ID NO:79. In some embodiments, an antibody is provided that specifically binds to Claudin18.2 competitively with an anti-Claudin18.2 single domain antibody comprising the amino acid sequence of SEQ ID NO:80. In some embodiments, an antibody is provided that specifically binds to Claudin18.2 competitively with an anti-Claudin18.2 single domain antibody comprising the amino acid sequence of SEQ ID NO:81. In some embodiments, an antibody is provided that specifically binds to Claudin18.2 competitively with an anti-Claudin18.2 single domain antibody comprising the amino acid sequence of SEQ ID NO:82. In some embodiments, an antibody is provided that specifically binds to Claudin18.2 competitively with an anti-Claudin18.2 single domain antibody comprising the amino acid sequence of SEQ ID NO:83. In some embodiments, an antibody is provided that specifically binds to Claudin18.2 competitively with an anti-Claudin18.2 single domain antibody comprising the amino acid sequence of SEQ ID NO: 84. In some embodiments, an antibody is provided that specifically binds to Claudin18.2 competitively with an anti-Claudin18.2 single domain antibody comprising the amino acid sequence of SEQ ID NO:85.

In some embodiments, the Claudin18.2 binding moiety comprises an antibody . In some embodiments, the Claudin18.2 binding moiety comprises a camelid, chimeric or humanized antibody. In some embodiments, the Claudin18.2 binding moiety is a single domain antibody. In some embodiments, the Claudin18.2 binding moiety is an antigen binding fragment or a variable region of an sdAb. In some embodiments, the Claudin18.2 binding moiety comprises an antibody having a CDR1, a CDR2, and/or a CDR3 from an sdAb described herein. In some embodiments, a Claudin18.2 binding moiety comprises a variant of an anti-Claudin18.2 antibody described herein. In some embodiments, a variant of the anti-Claudin18.2 antibody comprises one to thirty conservative amino acid substitutions. In some embodiments, a variant of the anti-Claudin18.2 antibody comprises one to twenty-five conservative amino acid substitutions. In some embodiments, a variant of the anti-Claudin18.2 antibody comprises one to twenty conservative amino acid substitutions. In some embodiments, a variant of the anti-Claudin18.2 antibody comprises one to fifteen conservative amino acid substitutions. In some embodiments, a variant of the anti-Claudin18.2 antibody comprises one to ten conservative amino acid substitutions. In some embodiments, a variant of the anti-Claudin18.2 antibody comprises one to five conservative amino acid substitutions. In some embodiments, a variant of the anti-Claudin18.2 antibody comprises one to three conservative amino acid substitutions. In some embodiments, the conservative amino acid substitution is in a VHH CDR of the antibody. In some embodiments, the conservative amino acid substitution is not in a VHH CDR of the antibody. In some embodiments, the conservative amino acid substitution is in a framework region of the antibody.

In some embodiments, there is provided an anti-Claudin18.2 antibody or antigen binding protein comprising any one of the anti-Claudin18.2 sdAbs described above. In some embodiments, the anti-Claudin18.2 antibody is a monoclonal antibody, including a camelid, chimeric, humanized or human antibody. In some embodiments, the anti-Claudin18.2 antibody is an antibody fragment, e.g., a VHH fragment. In some embodiments, the anti-Claudin18.2 antibody is a full-length heavy-chain only antibody comprising an Fc region of any antibody class or isotype, such as IgG1 or IgG4. In some embodiments, the Fc region has reduced or minimized effector function, if needed. Other exemplary Claudin18.2 binding molecules are described in more detail in the following sections. In some embodiments, provided herein is an isolated nucleic acid encoding any of the Claudin18.2 binding moieties provided herein. More detailed description regarding nucleic acid sequences and vectors are provided below.

In some embodiments, the anti-Claudin18.2 antibody (such as anti-Claudin18.2 single domain antibody) or antigen binding protein according to any of the above embodiments may incorporate any of the features, singly or in combination, as described in Sections 5.2.2 to 5.2.7 below. Various aspects mentioned above are also further described with more details in the following sections.

### 5.2.2. Humanized Single Domain Antibodies

The single domain antibodies described herein include humanized single domain antibodies. General strategies to humanize single domain antibodies from Camelidae species have been described (see, e.g., Vincke et al., J. Biol. Chem., 284(5):3273-3284 (2009)) and may be useful for producing humanized VHH domains as disclosed herein. The design of humanized single domain antibodies from Camelidae species may include the hallmark residues in the VHH, such as residues 11, 37, 44, 45 and 47 (residue numbering according to Kabat) (Muyldermans, Reviews Mol Biotech 74:277-302 (2001).

Humanized antibodies, such as the humanized single domain antibodies disclosed herein can also be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, Molecular Immunology 28(⅘):489-498 (1991); Studnicka et al., Protein Engineering 7(6):805-814 (1994); and Roguska et al., PNAS 91:969-973 (1994)), chain shuffling (U.S. Pat. No. 5,565,332), and techniques disclosed in, e.g., U.S. Pat. No. 6,407,213, U.S. Pat. No. 5,766,886, WO 93 17105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16): 10678-84 (1997), Roguska et al., Protein Eng. 9(10):895 904 (1996), Couto et al., Cancer Res. 55 (23 Supp):5973s- 5977 s (1995), Couto et al., Cancer Res. 55(8):1717-22 (1995), Sandhu JS, Gene 150(2):409-10 (1994), and Pedersen et al., J. Mol. Biol. 235(3):959-73 (1994). See also U.S. Pat. Pub. No. US 2005/0042664 A1 (Feb. 24, 2005), each of which is incorporated by reference herein in its entirety.

In some embodiments, single domain antibodies provided herein can be humanized single domain antibodies that bind to Claudin18.2, including human Claudin18.2. For example, humanized single domain antibodies of the present disclosure may comprise one or more CDRs set forth in SEQ ID NOs: 38-51 and 77-85. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., Nature 321 :522-25 (1986); Riechmann et al., Nature 332:323-27 (1988); and Verhoeyen et al., Science 239:1534-36 (1988)), by substituting hypervariable region sequences for the corresponding sequences of a human antibody. In a specific embodiment, humanization of the single domain antibody provided herein is performed as described in Section 6 below.

In some cases, the humanized antibodies are constructed by CDR grafting, in which the amino acid sequences of the CDRs of the parent non-human antibody are grafted onto a human antibody framework. For example, Padlan et al. determined that only about one third of the residues in the CDRs actually contact the antigen, and termed these the “specificity determining residues,” or SDRs (Padlan et al., FASEB J. 9:133-39 (1995)). In the technique of SDR grafting, only the SDR residues are grafted onto the human antibody framework (see, e.g., Kashmiri et al., Methods 36:25-34 (2005)).

The choice of human variable domains to be used in making the humanized antibodies can be important to reduce antigenicity. For example, according to the so-called “best-fit” method, the sequence of the variable domain of a non-human antibody is screened against the entire library of known human variable-domain sequences. The human sequence that is closest to that of the non-human antibody may be selected as the human framework for the humanized antibody (Sims et al., J. Immunol. 151:2296-308 (1993); and Chothia et al., J. Mol. Biol. 196:901-17 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA 89:4285-89 (1992); and Presta et al., J. Immunol. 151:2623-32 (1993)). In some cases, the framework is derived from the consensus sequences of the most abundant human subclasses, V_{L}6 subgroup I (V_{L}6I) and V_{H} subgroup III (V_{H}III). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., J. Immunol. 169:1119-25 (2002)).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13:819-24 (2002)), Modeller (Sali and Blundell, J. Mol. Biol. 234:779-815 (1993)), and Swiss PDB Viewer (Guex and Peitsch, Electrophoresis 18:2714-23 (1997)). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., Mol. Immunol. 44: 1986-98 (2007)).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, Nat. Biotechnol. 23:1105-16 (2005); Dufner et al., Trends Biotechnol. 24:523-29 (2006); Feldhaus et al., Nat. Biotechnol. 21:163-70 (2003); and Schlapschy et al., Protein Eng. Des. Sel. 17:847-60 (2004)).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, J. Mol. Biol. 224:487-99 (1992)), or from the more limited set of target residues identified by Baca et al. J. Biol. Chem. 272:10678-84 (1997).

In FR shuffling, whole FRs are combined with the non-human CDRs instead of creating combinatorial libraries of selected residue variants (see, e.g., Dall’ Acqua et al., Methods 36:43-60 (2005)). A one-step FR shuffling process may be used. Such a process has been shown to be efficient, as the resulting antibodies exhibited improved biochemical and physicochemical properties including enhanced expression, increased affinity, and thermal stability (see, e.g., Damschroder et al., Mol. Immunol. 44:3049-60 (2007)).

The “humaneering” method is based on experimental identification of essential minimum specificity determinants (MSDs) and is based on sequential replacement of non-human fragments into libraries of human FRs and assessment of binding. This methodology typically results in epitope retention and identification of antibodies from multiple subclasses with distinct human V-segment CDRs.

The “human engineering” method involves altering a non-human antibody or antibody fragment by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody’s folding. The particular human amino acid residue to be substituted at a given position (e.g., low or moderate risk) of a non-human antibody sequence can be selected by aligning an amino acid sequence from the non-human antibody’s variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering 7:805-14 (1994); U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody.

A deimmunized antibody is an antibody in which T-cell epitopes have been removed. Methods for making deimmunized antibodies have been described. See, e.g., Jones et al., Methods Mol Biol. 525:405-23 (2009), xiv, and De Groot et al., Cell. Immunol. 244:148-153(2006)). Deimmunized antibodies comprise T-cell epitope-depleted variable regions and human constant regions. Briefly, variable regions of an antibody are cloned and T-cell epitopes are subsequently identified by testing overlapping peptides derived from the variable regions of the antibody in a T cell proliferation assay. T cell epitopes are identified via in silico methods to identify peptide binding to human MHC class II. Mutations are introduced in the variable regions to abrogate binding to human MHC class II. Mutated variable regions are then utilized to generate the deimmunized antibody.

In some more specific embodiments, the humanized single domain antibodies are produced according the methods exemplified in Section 6 below. In some specific embodiments, the humanized single domain antibodies provided herein comprise SEQ ID NOs:77-85.

### 5.2.3. Single Domain Antibody Variants

In some embodiments, amino acid sequence modification(s) of the single domain antibodies that bind to Claudin18.2 described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the single domain antibodies that bind to Claudin18.2 described herein, it is contemplated that variants of the single domain antibodies that bind to Claudin18.2 described herein can be prepared. For example, single domain antibody variants can be prepared by introducing appropriate nucleotide changes into the encoding DNA, and/or by synthesis of the desired antibody or polypeptide. Those skilled in the art who appreciate that amino acid changes may alter post-translational processes of the single domain antibody.

### Chemical Modifications

In some embodiments, the single domain antibodies provided herein are chemically modified, for example, by the covalent attachment of any type of molecule to the single domain antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, or conjugation to one or more immunoglobulin domains (e.g., Fc or a portion of an Fc). Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

When the single domain antibody provided herein is fused to an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region . See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the binding molecules provided herein may be made in order to create variants with certain improved properties.

In other embodiments, when the single domain antibody provided herein is fused to an Fc region, antibody variants provided herein may have a carbohydrate structure that lacks fucose attached (directly or indirectly) to said Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ± 3 amino acids upstream or downstream of position 297, i.e., between positions 294 and 300, due to minor sequence variations in antibodies. Such fucosylation variants may have improved ADCC function. See, e.g., U.S. Pat. Publication Nos. US 2003/0157108 and US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); U.S. Pat. Application No. US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

The binding molecules comprising a single domain antibody provided herein are further provided with bisected oligosaccharides, e.g., in which a biantennary oligosaccharide attached to the Fc region is bisected by G1cNAc. Such variants may have reduced fucosylation and/or improved ADCC function. Examples of such variants are described, e.g., in WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such variants may have improved CDC function. Such variants are described, e.g., in WO 1997/30087; WO 1998/58964; and WO 1999/22764.

In molecules that comprise the present single domain antibody and an Fc region, one or more amino acid modifications may be introduced into the Fc region, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the present application contemplates variants that possesses some but not all effector functions, which make it a desirable candidate for applications in which the half life of the binding molecule in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the binding molecule lacks FcyR binding (hence likely lacking ADCC activity), but retains FcRn binding ability. Non-limiting examples of in vitro assays to assess ADCC activity of a molecule of interest is described in U.S. Pat. No. 5,500,362 (see, e.g. Hellstrom, I. et al. Proc. Nat’lAcad. Sci. USA 83:7059-7063 (1986)) and Hellstrom, I et al., Proc. Nat’lAcad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96^{®} non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat’lAcad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M.S. et al., Blood 101:1045-1052 (2003); and Cragg, M.S. and M.J. Glennie, Blood 103:2738-2743 (2004)). FcRn binding and in vivo clearance/half life determinations can also be performed using methods known in the art (see, e.g., Petkova, S.B. et al., Int′l. Immunol. 18(12): 1759-1769 (2006)).

Binding molecules with reduced effector function include those with substitution of one or more of Fc region residues 238, 265, 269, 270, 297, 327 and 329 (U.S. Patent No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, a variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Binding molecules with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those molecules comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. No. 5,648,260; U.S. Pat. No. 5,624,821; and WO 94/29351 concerning other examples of Fc region variants.

In some embodiments, it may be desirable to create cysteine engineered antibodies, in which one or more residues of an antibody are substituted with cysteine residues. In some embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein.

### Substitutions, Deletions, or Insertions

Variations may be a substitution, deletion, or insertion of one or more codons encoding the single domain antibody or polypeptide that results in a change in the amino acid sequence as compared with the original antibody or polypeptide. Sites of interest for substitutional mutagenesis include the CDRs and FRs.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Single domain antibodies generated by conservative amino acid substitutions are included in the present disclosure. In a conservative amino acid substitution, an amino acid residue is replaced with an amino acid residue having a side chain with a similar charge. As described above, families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties. Exemplary substitutions are shown in Table 3 below.

TABLE 3

Amino Acid Substitutions

| | | | |
|---|---|---|---|
| Original Residue | Exemplary Substitutions | Original Residue | Exemplary Substitutions |
| Ala (A) | Val; Leu; Ile | Leu (L) | Norleucine; Ile; Val; Met; Ala; Phe |
| Arg (R) | Lys; Gln; Asn | Lys (K) | Arg; Gln; Asn |
| Asn (N) | Gln; His; Asp, Lys; Arg | Met (M) | Leu; Phe; Ile |
| Asp (D) | Glu, Asn | Phe (F) | Trp; Leu; Val; Ile; Ala; Tyr |
| Cys (C) | Ser; Ala | Pro (P) | Ala |
| Gln (Q) | Asn; Glu | Ser (S) | Thr |
| Glu (E) | Asp; Gln | Thr (T) | Val; Ser |
| Gly (G) | Ala | Trp (W) | Tyr; Phe |
| His (H) | Asn; Gin; Lys; Arg | Tyr (Y) | Trp; Phe; Thr; Ser |
| Ile (I) | Leu; Val; Met; Ala; Phe; Norleucine | Val (V) | Ile; Leu; Met; Phe; Ala; Norleucine |

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. For example, any cysteine residue not involved in maintaining the proper conformation of the single domain antibody also may be substituted, for example, with another amino acid, such as alanine or serine, to improve the oxidative stability of the molecule and to prevent aberrant crosslinking. Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant antibody or fragment thereof being tested for binding affinity. Affinity maturation by constructing and reselecting from secondary libraries has been described, e.g., in Hoogenboom et al. in Methods in Molecular Biology 178:1-37 (O′Brien et al., ed, Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. More detailed description regarding affinity maturation is provided in the section below.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments of the variant VHH sequences provided herein, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, 244: 1081-1085 (1989). In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

The variations can be made using methods known in the art such as oligonucleotide-mediated (site-directed) mutagenesis, alanine scanning, and PCR mutagenesis. Site-directed mutagenesis (see, e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the single domain antibody variant DNA.

In some embodiments, a variant of a Claudin18.2 binding moiety disclosed herein can retain the ability to recognize a target (e.g., Claudin 18.2) to a similar extent, the same extent, or to a higher extent, as the parent binding moiety. In some embodiments, the variant can be at least about 80%, about 85%, about 90%, about 91 %, about 92%, about 93%, about 94%, about 95%, about 96%, about 97%, about 98%, or about 99% identical in amino acid sequence to the parent binding moiety. In some embodiments, the variant can have an amino acid sequence that is at least about 80%, about 85%, about 90%, about 91 %, about 92%, about 93%, about 94%, about 95%, about 96%, about 97%, about 98%, or about 99% identical to the antibodies disclosed herein.

### 5.2.4. In Vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, Methods. Mol. Biol. 178:1-37 (2002); and Bradbury and Marks, J. Immunol. Methods 290:29-49 (2004).

In a yeast display system (see, e.g., Boder et al., Nat. Biotech. 15:553-57 (1997); and Chao et al., Nat. Protocols 1:755-68 (2006)), the antibody may be fused to the adhesion subunit of the yeast agglutinin protein Aga2p, which attaches to the yeast cell wall through disulfide bonds to Agalp. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the single chain antibody (e.g., scFv). Expression has been shown to correlate with the stability of the displayed protein, and thus antibodies can be selected for improved stability as well as affinity (see, e.g., Shusta et al., J. Mol. Biol. 292:949-56 (1999)). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells’ mating system to create combinatorial diversity estimated to be 10¹⁴ in size (see, e.g., U.S. Pat. Publication 2003/0186374; and Blaise etal., Gene 342:211-18 (2004)).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., Nucleic Acids Res. 34:e127 (2006)). In mRNA display, a covalent bond between antibody and mRNA is established using puromycin as an adaptor molecule (Wilson et al., Proc. Natl. Acad. Sci. USA 98:3750-55 (2001)).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In some embodiments, mammalian display systems may be used.

Diversity may also be introduced into the CDRs of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., J. Biol. Chem. 280:607-17 (2005)) or residues suspected of affecting affinity on experimental basis or structural reasons. Diversity may also be introduced by replacement of regions that are naturally diverse via DNA shuffling or similar techniques (see, e.g., Lu et al., J. Biol. Chem. 278:43496-507 (2003); U.S. Pat. Nos. 5,565,332 and 6,989,250). Alternative techniques target hypervariable loops extending into framework-region residues (see, e.g., Bond et al., J. Mol. Biol. 348:699-709 (2005)) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, single domain antibodies can be immobilized onto solid supports, columns, pins, or cellulose/poly (vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, Nature Biotechnology 23:1105-16 (2005); Quiroz and Sinclair, Revista Ingeneria Biomedia 4:39-51 (2010); and references therein.

### 5.2.5. Modifications of Single Domain Antibodies

Covalent modifications of single domain antibodies are included within the scope of the present disclosure. Covalent modifications include reacting targeted amino acid residues of a single domain antibody with an organic derivatizing agent that is capable of reacting with selected side chains or the N- or C- terminal residues of the single domain antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the single domain antibody included within the scope of this present disclosure include altering the native glycosylation pattern of the antibody or polypeptide as described above (see, e.g., Beck et al., Curr. Pharm. Biotechnol. 9:482-501 (2008); and Walsh, Drug Discov. Today 15:773-80 (2010)), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337. The single domain antibody that binds to Claudin18.2 of the disclosure may also be genetically fused or conjugated to one or more immunoglobulin constant regions or portions thereof (e.g., Fc) to extend half-life and/or to impart known Fc-mediated effector functions.

The single domain antibody that binds to Claudin18.2 of the present disclosure may also be modified to form chimeric molecules comprising the single domain antibody that binds to Claudin18.2 fused to another, heterologous polypeptide or amino acid sequence, for example, an epitope tag (see, e.g., Terpe, Appl. Microbiol. Biotechnol. 60:523-33 (2003)) or the Fc region of an IgG molecule (see, e.g., Aruffo, Antibody Fusion Proteins 221-42 (Chamow and Ashkenazi eds., 1999)). The single domain antibody that binds to Claudin18.2 may also be used to generate Claudin18.2 binding chimeric antigen receptor (CAR), as described in more detail below.

Also provided herein are fusion proteins comprising the single domain antibody that binds to Claudin18.2 of the disclosure and a heterologous polypeptide. In some embodiments, the heterologous polypeptide to which the antibody is genetically fused or chemically conjugated is useful for targeting the antibody to cells having cell surface-expressed Claudin18.2.

Also provided herein are panels of antibodies that bind to a Claudin18.2 antigen. In specific embodiments, the panels of antibodies have different association rates, different dissociation rates, different affinities for a Claudin18.2 antigen, and/or different specificities for a Claudin18.2 antigen. In some embodiments, the panels comprise or consist of about 10 to about 1000 antibodies or more. Panels of antibodies can be used, for example, in 96-well or 384-well plates, for assays such as ELISAs.

### 5.2.6. Preparation of Single Domain Antibodies

Methods of preparing single domain antibodies have been described. See, e.g., Els Pardon et al, Nature Protocol, 9(3): 674 (2014). Single domain antibodies (such as VHHs) may be obtained using methods known in the art such as by immunizing a Camelid species (such as camel or llama) and obtaining hybridomas therefrom, or by cloning a library of single domain antibodies using molecular biology techniques known in the art and subsequent selection by ELISA with individual clones of unselected libraries or by using phage display.

Single domain antibodies provided herein may be produced by culturing cells transformed or transfected with a vector containing a single domain antibody-encoding nucleic acids. Polynucleotide sequences encoding polypeptide components of the antibody of the present disclosure can be obtained using standard recombinant techniques. Desired polynucleotide sequences may be isolated and sequenced from antibody producing cells such as hybridomas cells or B cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells suitable for expressing antibodies of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. Antibodies produced by the host cells are purified using standard protein purification methods as known in the art.

Methods for antibody production including vector construction, expression, and purification are further described in Pluckthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherichia coli, in Antibody Expression and Production (Al-Rubeai ed., 2011); and Therapeutic Monoclonal Antibodies: From Bench to Clinic (An ed., 2009).

It is, of course, contemplated that alternative methods, which are well known in the art, may be employed to prepare anti-Claudin18.2 single domain antibodies. For instance, the appropriate amino acid sequence, or portions thereof, may be produced by direct peptide synthesis using solid-phase techniques (see, e.g., Stewart et al., Solid-Phase Peptide Synthesis (1969); and Merrifield, J. Am. Chem. Soc. 85:2149-54 (1963)). In vitro protein synthesis may be performed using manual techniques or by automation. Various portions of the anti-Claudin18.2 antibody may be chemically synthesized separately and combined using chemical or enzymatic methods to produce the desired anti-Claudin18.2 antibody. Alternatively, antibodies may be purified from cells or bodily fluids, such as milk, of a transgenic animal engineered to express the antibody, as disclosed, for example, in U.S. Pat. Nos. 5,545,807 and 5,827,690.

Specifically, the single domain antibodies, or other Claudin18.2 binders provided herein, can be generated by immunizing llamas, performing single B-cell sorting, undertaking V-gene extraction, cloning the Claudin18.2 binders, such as VHH-Fc fusions, and then performing small scale expression and purification. Additional screening of the single domain antibodies and other molecules that bind to Claudin18.2 can be performed, including one or more of selecting for ELISA-positive, BLI-positive, and K_{D} less than 100 nM. These selection criteria can be combined as described in Section 6 below. Additionally, individual VHH binders (and other molecules that bind to Claudin18.2) can be assayed for their ability to bind to cells expressing Claudin18.2. Such assay can be performed using FACS analysis with cells expressing Claudin18.2, and measuring the mean fluorescence intensity (MFI) of fluorescently-labeled VHH molecules. Various aspects mentioned above are described in more details below.

### Polyclonal Antibodies

Polyclonal antibodies are generally raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are independently lower alkyl groups. Examples of adjuvants which may be employed include Freund’s complete adjuvant and MPL-TDM adjuvant (monophosphoryl Lipid A, synthetic trehalose dicorynomycolate). The immunization protocol may be selected by one skilled in the art without undue experimentation.

For example, the animals are immunized against the antigen, immunogenic conjugates, or derivatives by combining, e.g., 100 µg or 5 µg of the protein or conjugate (for rabbits or mice, respectively) with 3 volumes of Freund’s complete adjuvant and injecting the solution intradermally at multiple sites. One month later, the animals are boosted with ⅕ to ⅒ the original amount of peptide or conjugate in Freund’s complete adjuvant by subcutaneous injection at multiple sites. Seven to fourteen days later, the animals are bled and the serum is assayed for antibody titer. Animals are boosted until the titer plateaus. Conjugates also can be made in recombinant cell culture as protein fusions. Also, aggregating agents such as alum are suitable to enhance the immune response.

### Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, an appropriate host animal is immunized to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986).

The immunizing agent will typically include the antigenic protein or a fusion variant thereof. Goding, Monoclonal Antibodies: Principles and Practice, Academic Press (1986), pp. 59-103. Immortalized cell lines are usually transformed mammalian cells. The hybridoma cells thus prepared are seeded and grown in a suitable culture medium that preferably contains one or more substances that inhibit the growth or survival of the unfused, parental myeloma cells. Preferred immortalized myeloma cells are those that fuse efficiently, support stable high-level production of antibody by the selected antibody-producing cells, and are sensitive to a medium such as HAT medium.

Culture medium in which hybridoma cells are growing is assayed for production of monoclonal antibodies directed against the antigen. The culture medium in which the hybridoma cells are cultured can be assayed for the presence of monoclonal antibodies directed against the desired antigen. Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Pliickthun, Immunol. Revs. 130:151-188 (1992).

In a further embodiment, antibodies can be isolated from antibody phage libraries generated using the techniques described in McCafferty et a/., Nature, 348:552-554 (1990). Clackson et al., Nature, 352:624-628 (1991) and Marks et al, J. Mol. Biol., 222:581-597 (1991). Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence (U.S. Pat. No. 4,816,567; Morrison, et al., Proc. Natl Acad. Sci. USA, 81:6851 (1984)), or by covalently joining to the coding sequence all or part of the coding sequence for a non-immunoglobulin polypeptide. Such non-immunoglobulin polypeptides can be substituted to create a chimeric bivalent antibody comprising one antigen-combining site having specificity for an antigen and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Recombinant Production in Prokaryotic Cells

Polynucleic acid sequences encoding the antibodies of the present disclosure can be obtained using standard recombinant techniques. Desired polynucleic acid sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to, an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector which can be used to transform susceptible host cells such as E. coli LE392.

The expression vector of the present application may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the present antibody by removing the promoter from the source DNA via restriction enzyme digestion and inserting the isolated promoter sequence into the vector of the present application. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the -galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleic acid sequences have been published, thereby enabling a skilled worker operably to ligate them to cistrons encoding the target peptide (Siebenlist et al. Cell 20: 269 (1980)) using linkers or adaptors to supply any required restriction sites.

In one aspect, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence can be substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

In some embodiments, the production of the antibodies according to the present disclosure can occur in the cytoplasm of the host cell, and therefore does not require the presence of secretion signal sequences within each cistron. Certain host strains (e.g., the E. coli trxB⁻ strains) provide cytoplasm conditions that are favorable for disulfide bond formation, thereby permitting proper folding and assembly of expressed protein subunits.

Prokaryotic host cells suitable for expressing the antibodies of the present disclosure include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In one embodiment, E. coli cells are used as hosts. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A(nmpc-fepE) degP41 kan^{R} (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon.

Typically the host cell should secrete minimal amounts of proteolytic enzymes, and additional protease inhibitors may desirably be incorporated in the cell culture.

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the antibodies of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol. The prokaryotic host cells are cultured at suitable temperatures and pHs.

If an inducible promoter is used in the expression vector of the present application, protein expression is induced under conditions suitable for the activation of the promoter. In one aspect of the present application, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods 263:133-147 (2002)). A variety of other inducers may be used, according to the vector construct employed, as is known in the art.

The expressed antibodies of the present disclosure are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale fed-batch fermentation procedures are available for production of recombinant proteins. To improve the production yield and quality of the antibodies of the present disclosure, various fermentation conditions can be modified. For example, the chaperone proteins have been demonstrated to facilitate the proper folding and solubility of heterologous proteins produced in bacterial host cells. Chen et al. J Bio Chem 274:19601-19605 (1999); U.S. Pat. No. 6,083,715; U.S. Pat. No. 6,027,888; Bothmann and Pluckthun, J. Biol. Chem. 275:17100-17105 (2000); Ramm and Pluckthun, J. Biol. Chem. 275:17106-17113 (2000); Arie et al., Mol. Microbiol. 39:199-210 (2001).

To minimize proteolysis of expressed heterologous proteins (especially those that are proteolytically sensitive), certain host strains deficient for proteolytic enzymes can be used for the present invention, as described in, for example, U.S. Pat. No. 5,264,365; U.S. Pat. No. 5,508,192; Hara et al., Microbial Drug Resistance, 2:63-72 (1996). E. coli strains deficient for proteolytic enzymes and transformed with plasmids overexpressing one or more chaperone proteins may be used as host cells in the expression system encoding the antibodies of the present application.

The antibodies produced herein can be further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75. Protein A immobilized on a solid phase for example can be used in some embodiments for immunoaffinity purification of binding molecules of the present disclosure. The solid phase to which Protein A is immobilized is preferably a column comprising a glass or silica surface, more preferably a controlled pore glass column or a silicic acid column. In some embodiments, the column has been coated with a reagent, such as glycerol, in an attempt to prevent nonspecific adherence of contaminants. The solid phase is then washed to remove contaminants non-specifically bound to the solid phase. Finally the antibodies of interest is recovered from the solid phase by elution.

### Recombinant Production in Eukaryotic Cells

For eukaryotic expression, the vector components generally include, but are not limited to, one or more of the following, a signal sequence, an origin of replication, one or more marker genes, and enhancer element, a promoter, and a transcription termination sequence.

A vector for use in a eukaryotic host may also an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available. The DNA for such precursor region can be ligated in reading frame to DNA encoding the antibodies of the present application.

Generally, the origin of replication component is not needed for mammalian expression vectors (the SV40 origin may typically be used only because it contains the early promoter).

Expression and cloning vectors may contain a selection gene, also termed a selectable marker. Selection genes may encode proteins that confer resistance to antibiotics or other toxins, e.g., ampicillin, neomycin, methotrexate, or tetracycline; complement auxotrophic deficiencies; or supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the antibodies of the present application. For example, cells transformed with the DHFR selection gene are first identified by culturing all of the transformants in a culture medium that contains methotrexate (Mtx), a competitive antagonist of DHFR. An exemplary appropriate host cell when wild-type DHFR is employed is the Chinese hamster ovary (CHO) cell line deficient in DHFR activity. Alternatively, host cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic.

Expression and cloning vectors usually contain a promoter that is recognized by the host organism and is operably linked to the nucleic acid encoding the desired polypeptide sequences. Eukaryotic genes have an AT-rich region located approximately 25 to 30 based upstream from the site where transcription is initiated. Another sequence found 70 to 80 bases upstream from the start of the transcription of many genes may be included. The 3′ end of most eukaryotic may be the signal for addition of the poly A tail to the 3′ end of the coding sequence. All of these sequences may be inserted into eukaryotic expression vectors.

Polypeptide transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding the antibodies of the present disclosure by higher eukaryotes is often increased by inserting an enhancer sequence into the vector. Many enhancer sequences are now known from mammalian genes (globin, elastase, albumin, α-fetoprotein, and insulin). Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

Expression vectors used in eukaryotic host cells (yeast, fungi, insect, plant, animal, human, or nucleated cells from other multicellular organisms) also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region.

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells can be transformed with the above-described expression or cloning vectors for antibodies production and cultured in conventional nutrient media modified as appropriate for inducing promoters, selecting transformants, or amplifying the genes encoding the desired sequences.

The host cells used to produce the antibodies of the present application may be cultured in a variety of media. Commercially available media such as Ham’s F10 (Sigma), Minimal Essential Medium ((MEM), (Sigma), RPMI-1640 (Sigma), and Dulbecco’s Modified Eagle’s Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibodies can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, and affinity chromatography, with affinity chromatography being the preferred purification technique. The matrix to which the affinity ligand is attached is most often agarose, but other matrices are available. Mechanically stable matrices such as controlled pore glass or poly (styrene-divinyl) benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered. Following any preliminary purification step(s), the mixture comprising the antibody of interest and contaminants may be subjected to low pH hydrophobic interaction chromatography.

### 5.2.7. Binding Molecules Comprising the Single Domain Antibodies

In another aspect, provided herein is a binding molecule comprising a single domain antibody (e.g., a VHH domain against Claudin18.2) provided herein. In addition to chimeric antigen receptors (CARs) provided herein as described in Section 5.3 below, in some embodiments, a single domain antibody against Claudin18.2 provided herein is part of other binding molecules. Exemplary binding molecules of the present disclosure are described herein.

### Fusion Protein

In various embodiments, the single domain antibody provided herein can be genetically fused or chemically conjugated to another agent, for example, protein-based entities. The single domain antibody may be chemically-conjugated to the agent, or otherwise non-covalently conjugated to the agent. The agent can be a peptide or an antibody (or a fragment thereof).

Thus, in some embodiments, provided herein are single domain antibodies (e.g., VHH domains) that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, about 100, about 150, about 200, about 250, about 300, about 350, about 400, about 450 or about 500 amino acids, or over 500 amino acids) to generate fusion proteins, as well as uses thereof. In particular, provided herein are fusion proteins comprising an antigen-binding fragment of the single domain antibody provided herein (e.g., CDR1, CDR2, and/or CDR3) and a heterologous protein, polypeptide, or peptide.

Moreover, antibodies provided herein can be fused to marker or “tag” sequences, such as a peptide, to facilitate purification. In specific embodiments, the marker or tag amino acid sequence is a hexa-histidine peptide, hemagglutinin (“HA”) tag, and “FLAG” tag.

Methods for fusing or conjugating moieties (including polypeptides) to antibodies are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., Immunol. Rev. 62:119-58 (1982); U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88: 10535-39 (1991); Traunecker et al., Nature, 331:84-86 (1988); Zheng et al., J. Immunol. 154:5590-600 (1995); and Vil et al., Proc. Natl. Acad. Sci. USA 89:11337-41 (1992)).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the single domain antibodies as provided herein, including, for example, antibodies with higher affinities and lower dissociation rates (see, e.g., U.S. Pat. Nos. 5,605,793; 5,811,238; 5,830,721; 5,834,252; and 5,837,458; Patten et al., Curr. Opinion Biotechnol. 8:724-33 (1997); Harayama, Trends Biotechnol. 16(2):76-82 (1998); Hansson et al., J. Mol. Biol. 287:265-76 (1999); and Lorenzo and Blasco, Biotechniques 24(2):308-13 (1998)). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

In some embodiments, a single domain antibody provided herein (e.g., VHH domain) is conjugated to a second antibody to form an antibody heteroconjugate.

In various embodiments, the single domain antibody is genetically fused to the agent. Genetic fusion may be accomplished by placing a linker (e.g., a polypeptide) between the single domain antibody and the agent. The linker may be a flexible linker.

In various embodiments, the single domain antibody is genetically conjugated to a therapeutic molecule, with a hinge region linking the single domain antibody to the therapeutic molecule.

Also provided herein are methods for making the various fusion proteins provided herein. The various methods described in Section 5.2.6 above may also be utilized to make the fusion proteins provided herein.

In a specific embodiment, the fusion protein provided herein is recombinantly expressed. Recombinant expression of a fusion protein provided herein may require construction of an expression vector containing a polynucleotide that encodes the protein or a fragment thereof. Once a polynucleotide encoding a protein provided herein or a fragment thereof has been obtained, the vector for the production of the molecule may be produced by recombinant DNA technology using techniques well-known in the art. Thus, methods for preparing a protein by expressing a polynucleotide containing an encoding nucleotide sequence are described herein. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding a fusion protein provided herein, or a fragment thereof, or a CDR, operably linked to a promoter.

The expression vector can be transferred to a host cell by conventional techniques and the transfected cells are then cultured by conventional techniques to produce a fusion protein provided herein. Thus, also provided herein are host cells containing a polynucleotide encoding a fusion protein provided herein or fragments thereof operably linked to a heterologous promoter.

A variety of host-expression vector systems may be utilized to express the fusion protein provided herein. Such host-expression systems represent vehicles by which the coding sequences of interest may be produced and subsequently purified, but also represent cells which may, when transformed or transfected with the appropriate nucleotide coding sequences, express a fusion protein provided herein in situ. These include but are not limited to microorganisms such as bacteria (e.g., E. coli and B. subtilis) transformed with recombinant bacteriophage DNA, plasmid DNA or cosmid DNA expression vectors containing coding sequences; yeast (e.g., Saccharomyces Pichia) transformed with recombinant yeast expression vectors containing coding sequences; insect cell systems infected with recombinant virus expression vectors (e.g., baculovirus) containing coding sequences; plant cell systems infected with recombinant virus expression vectors (e.g., cauliflower mosaic virus, CaMV, tobacco mosaic virus, TMV) or transformed with recombinant plasmid expression vectors (e.g., Ti plasmid) containing coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5 K promoter). Bacterial cells such as Escherichia coli, or, eukaryotic cells, especially for the expression of whole recombinant antibody molecule, can be used for the expression of a recombinant fusion protein. For example, mammalian cells such as Chinese hamster ovary cells (CHO), in conjunction with a vector such as the major intermediate early gene promoter element from human cytomegalovirus is an effective expression system for antibodies or variants thereof. In a specific embodiment, the expression of nucleotide sequences encoding the fusion proteins provided herein is regulated by a constitutive promoter, inducible promoter or tissue specific promoter.

In bacterial systems, a number of expression vectors may be advantageously selected depending upon the use intended for the fusion protein being expressed. For example, when a large quantity of such a fusion protein is to be produced, for the generation of pharmaceutical compositions of a fusion protein, vectors which direct the expression of high levels of fusion protein products that are readily purified may be desirable. Such vectors include, but are not limited to, the E. coli expression vector pUR278 (Ruther et al., EMBO 12:1791 (1983)), in which the coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region El or E3) will result in a recombinant virus that is viable and capable of expressing the fusion protein in infected hosts (e.g., see Logan & Shenk, Proc. Natl. Acad. Sci. USA 8 1:355-359 (1984)). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells.

For long-term, high-yield production of recombinant proteins, stable expression can be utilized. For example, cell lines which stably express the fusion proteins may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the fusion protein. Such engineered cell lines may be particularly useful in screening and evaluation of compositions that interact directly or indirectly with the binding molecule.

A number of selection systems may be used, including but not limited to, the herpes simplex virus thymidine kinase (Wigler et al., Cell 11:223 (1977)), hypoxanthineguanine phosphoribosyltransferase (Szybalska & Szybalski, Proc. Natl. Acad. Sci. USA 48:202 (1992)), and adenine phosphoribosyltransferase (Lowy et al., Cell 22:8-17 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O′Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIB TECH 11(5):155-2 15 (1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., J. Mol. Biol. 150:1 (1981), which are incorporated by reference herein in their entireties.

The expression level of a fusion protein can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing a fusion protein is amplifiable, increase in the level of inhibitor present in culture of host cell will increase the number of copies of the marker gene. Since the amplified region is associated with the fusion protein gene, production of the fusion protein will also increase (Crouse et al., Mol. Cell. Biol. 3:257 (1983)).

The host cell may be co-transfected with multiple expression vectors provided herein. The vectors may contain identical selectable markers which enable equal expression of respective encoding polypeptides. Alternatively, a single vector may be used which encodes, and is capable of expressing multiple polypeptides. The coding sequences may comprise cDNA or genomic DNA.

Once a fusion protein provided herein has been produced by recombinant expression, it may be purified by any method known in the art for purification of a polypeptide (e.g., an immunoglobulin molecule), for example, by chromatography (e.g., ion exchange, affinity, particularly by affinity for the specific antigen after Protein A, sizing column chromatography, and Kappa select affinity chromatography), centrifugation, differential solubility, or by any other standard technique for the purification of proteins. Further, the fusion protein molecules provided herein can be fused to heterologous polypeptide sequences described herein or otherwise known in the art to facilitate purification.

### Immunoconjugates

In some embodiments, the present disclosure also provides immunoconjugates comprising any of the antibodies (such as anti-Claudin18.2 single domain antibodies) described herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some embodiments, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Patent Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Patent No. 6,630,579); methotrexate; vindesine; a taxane such as docetaxel, paclitaxel, larotaxel, tesetaxel, and ortataxel; a trichothecene; and CC1065.

In some embodiments, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In some embodiments, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

The immunuoconjugates or ADCs herein contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

In other embodiments, antibodies provided herein are conjugated or recombinantly fused, e.g., to a diagnostic molecule. Such diagnosis and detection can be accomplished, for example, by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, 225Acy-emitting, Auger-emitting, β-emitting, an alpha-emitting or positron-emitting radioactive isotope.

### 5.3. Chimeric Antigen Receptors

One aspect of the present application provides a chimeric antigen receptor (CAR) comprising an extracellular antigen binding domain comprising a Claudin18.2 binding moiety described herein, such as an sdAb or an antigen binding fragments thereof (e.g., a VHH domain). Any one of the anti-Claudin18.2 sdAbs or antigen binding fragments described above can be used in the CARs described herein. Exemplary CARs comprising the present VHH domains (i.e., VHH-based CARs) are illustrated and their superior effects are demonstrated as described in Section 6 below.

The CAR comprises (a) an extracellular antigen binding domain comprising an anti-Claudin18.2 sdAb or an antigen binding fragment thereof; (b) a transmembrane domain; and (c) an intracellular signaling domain. In some embodiments, the anti-Claudin18.2 sdAb can be camelid, chimeric or humanized. In some embodiments, the CAR comprises one or more anti-Claudin18.2 sdAbs or antigen binding fragments thereof. Each components and additional regions are described in more detail below.

### 5.3.1. Extracellular Antigen Binding Domain

The extracellular antigen binding domain of the CARs described herein comprises one or more (such as any one of 1, 2, 3, 4, 5, 6 or more) single domain antibodies. The single domain antibodies can be fused to each other directly via peptide bonds, or via peptide linkers.

### Single Domain Antibodies

The CARs of the present disclosure comprise an extracellular antigen binding domain comprising one or more single domain antibodies. The sdAbs may be of the same or different origins, and of the same or different sizes. Exemplary sdAbs include, but are not limited to, heavy chain variable domains from heavy-chain only antibodies (e.g., VHH or V_{NAR}), binding molecules naturally devoid of light chains, single domains (such as V_{H} or V_{L}) derived from conventional 4-chain antibodies, humanized heavy-chain only antibodies, human single domain antibodies produced by transgenic mice or rats expressing human heavy chain segments, and engineered domains and single domain scaffolds other than those derived from antibodies. Any sdAbs known in the art or developed by the present disclosure, including the single domain antibodies described above in the present disclosure, may be used to construct the CARs described herein. The sdAbs may be derived from any species including, but not limited to mouse, rat, human, camel, llama, lamprey, fish, shark, goat, rabbit, and bovine. Single domain antibodies contemplated herein also include naturally occurring single domain antibody molecules from species other than Camelidae and sharks.

In some embodiments, the sdAb is derived from a naturally occurring single domain antigen binding molecule known as heavy chain antibody devoid of light chains (also referred herein as “heavy chain only antibodies”). Such single domain molecules are disclosed in WO 94/04678 and Hamers-Casterman, C. et al., Nature 363:446-448 (1993), for example. For clarity reasons, the variable domain derived from a heavy chain molecule naturally devoid of light chain is known herein as a VHH to distinguish it from the conventional V_{H} of four chain immunoglobulins. Such a VHH molecule can be derived from antibodies raised in Camelidae species, for example, camel, llama, vicuna, dromedary, alpaca and guanaco. Other species besides Camelidae may produce heavy chain molecules naturally devoid of light chain, and such VHHs are within the scope of the present disclosure. In addition, humanized versions of VHHs as well as other modifications and variants are also contemplated and within the scope of the present disclosure.

VHH molecules from Camelids are about 10 times smaller than IgG molecules. They are single polypeptides and can be very stable, resisting extreme pH and temperature conditions. Moreover, they can be resistant to the action of proteases which is not the case for conventional 4-chain antibodies. Furthermore, in vitro expression of VHHs produces high yield, properly folded functional VHHs. In addition, antibodies generated in Camelids can recognize epitopes other than those recognized by antibodies generated in vitro through the use of antibody libraries or via immunization of mammals other than Camelids (see, for example, WO9749805). As such, multispecific or multivalent CARs comprising one or more VHH domains may interact more efficiently with targets than multispecific or multivalent CARs comprising antigen binding fragments derived from conventional 4-chain antibodies. Since VHHs are known to bind into “unusual” epitopes such as cavities or grooves, the affinity of CARs comprising such VHHs may be more suitable for therapeutic treatment than conventional multispecific polypeptides.

In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov, Protein Sci. 14:2901-2909 (2005).

In some embodiments, the sdAb is recombinant, CDR-grafted, humanized, camelized, de-immunized and/or in vitro generated (e.g., selected by phage display). In some embodiments, the amino acid sequence of the framework regions may be altered by “camelization” of specific amino acid residues in the framework regions. Camelization refers to the replacing or substitution of one or more amino acid residues in the amino acid sequence of a (naturally occurring) V_{H} domain from a conventional 4-chain antibody by one or more of the amino acid residues that occur at the corresponding position(s) in a VHH domain of a heavy chain antibody. This can be performed in a manner known in the field, which will be clear to the skilled person. Such “camelizing” substitutions are preferably inserted at amino acid positions that form and/or are present at the V_{H}-V_{L} interface, and/or at the so-called Camelidae hallmark residues, as defined herein (see for example WO 94/04678, Davies and Riechmann FEBS Letters 339: 285-290 (1994); Davies and Riechmann, Protein Engineering 9 (6): 531-537 (1996); Riechmann, J. Mol. Biol. 259: 957-969 (1996); and Riechmann and Muyldermans, J. Immunol. Meth. 231: 25-38 (1999)).

In some embodiments, the sdAb is a human single domain antibody produced by transgenic mice or rats expressing human heavy chain segments. See, e.g., US20090307787, U.S. Pat. No. 8,754,287, US20150289489, US20100122358, and WO2004049794. In some embodiments, the sdAb is affinity matured.

In some embodiments, naturally occurring VHH domains against a particular antigen or target, can be obtained from (naïve or immune) libraries of Camelid VHH sequences. Such methods may or may not involve screening such a library using said antigen or target, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from (naïve or immune) VHH libraries may be used, such as VHH libraries obtained from (naive or immune) VHH libraries by techniques such as random mutagenesis and/or CDR shuffling, as for example described in WO 00/43507.

In some embodiments, the single domain antibodies are generated from conventional four-chain antibodies. See, for example, EP 0 368 684; Ward et al., Nature, 341 (6242): 544-6 (1989); Holt et al., Trends Biotechnol., 21(11):484-490 (2003); WO 06/030220; and WO 06/003388.

In some embodiments, the extracellular antigen binding domain provided herein comprises at least one binding domain, and the at least one binding domain comprises a single domain antibody that binds to Claudin18.2 as provided herein, e.g., the anti-Claudin18.2 single domain antibodies described in Section 5.2 above.

In some embodiments, provided herein is a CAR comprising a polypeptide comprising: (a) an extracellular antigen binding domain comprising an anti-Claudin18.2 sdAb; (b) a transmembrane domain; and (c) an intracellular signaling domain, wherein the anti-Claudin18.2 sdAb is an anti-Claudin18.2 sdAb as described in Section 5.2 above, including, e.g., the VHH domains in Table 2 and those having one, two or all three CDRs in any of those VHH domains in Table 2. In some embodiments, the anti-Claudin18.2 sdAb is camelid, chimeric, human, or humanized.

In some embodiments, provided herein is a CAR comprising a polypeptide comprising: (a) an extracellular antigen binding domain comprising an anti-Claudin18.2 sdAb; (b) a transmembrane domain; and (c) an intracellular signaling domain, wherein the anti-Claudin18.2 sdAb comprises the amino acid sequence of SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:77, SEQ ID NO:78, SEQ ID NO:79, SEQ ID NO:80, SEQ ID NO:81, SEQ ID NO:82, SEQ ID NO:83, SEQ ID NO:84, or SEQ ID NO:85. In other embodiments, provided herein is a CAR comprising a polypeptide comprising: (a) an extracellular antigen binding domain comprising an anti-Claudin18.2 sdAb; (b) a transmembrane domain; and (c) an intracellular signaling domain, wherein the anti-Claudin18.2 sdAb comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identify to the amino acid sequence of SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:77, SEQ ID NO:78, SEQ ID NO:79, SEQ ID NO:80, SEQ ID NO:81, SEQ ID NO:82, SEQ ID NO:83, SEQ ID NO:84, or SEQ ID NO:85.

In other embodiments, the extracellular antigen binding domain comprises two or more antigen binding domains. Among these two or more antigen binding domains, at least one is a VHH that binds to Claudin18.2 as provided herein. In some embodiments, the one or more additional binding domain(s) is/are also VHH(s) that bind(s) to Claudin18.2. In other embodiments, the one or more additional binding domain(s) bind(s) to one or more additional different antigen(s), e.g., 1, 2, 3, 4 or more additional single domain antibody binding regions (sdAbs) targeting one or more additional different antigen(s).

In some embodiments, provided herein is a multivalent (such as bivalent and trivalent) CAR comprising a polypeptide comprising: (a) an extracellular antigen binding domain comprising two or more single domain antibodies (sdAbs) specifically binding to Claudin18.2; (b) a transmembrane domain; and (c) an intracellular signaling domain. In some embodiments, the extracellular antigen binding domain comprises two single domain antibodies (sdAbs) specifically binding to Claudin18.2 provided herein. In other embodiments, the extracellular antigen binding domain comprises three single domain antibodies (sdAbs) specifically binding to Claudin18.2 provided herein. In some embodiments, the two or more anti-Claudin18.2 sdAbs are selected from those anti-Claudin18.2 sdAbs described in Section 5.2 above, including, e.g., the VHH domains in Table 2 and those having one, two or all three CDRs in any of those VHH domains in Table 2. In some embodiments, the anti-Claudin18.2 sdAb is camelid, chimeric, human, or humanized. In some embodiments, the two or more anti-Claudin18.2 sdAbs are each independently selected from anti-Claudin18.2 sdAbs comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identify to the amino acid sequence of SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:77, SEQ ID NO:78, SEQ ID NO:79, SEQ ID NO:80, SEQ ID NO:81, SEQ ID NO:82, SEQ ID NO:83, SEQ ID NO:84, or SEQ ID NO:85.

In other embodiments, provided herein is a multispecific (such as bispecific and trispecific) CAR comprising a polypeptide comprising: (a) an extracellular antigen binding domain comprising a first single domain antibody (sdAb) specifically binding to Claudin18.2; (b) a transmembrane domain; and (c) an intracellular signaling domain. In some embodiments, the CAR further comprises a second single domain antibody (sdAb) specifically binding to a second antigen (such as a second tumor antigen). In some embodiments, the CAR further comprises a second single domain antibody (sdAb) specifically binding to a second antigen (such as a second tumor antigen); and a third single domain antibody (sdAb) specifically binding to a third antigen (such as a third tumor antigen).

In some embodiments, the additional antigen(s) targeted by the CARs of the present disclosure are cell surface molecules. The single domain antibodies may be chosen to recognize an antigen that acts as a cell surface marker on target cells associated with a special disease state. In some embodiments, the antigen is a tumor antigen. Tumors express a number of proteins that can serve as a target antigen for an immune response, particularly T cell mediated immune responses. The antigens targeted by the CAR may be antigens on a single diseased cell or antigens that are expressed on different cells that each contribute to the disease. The antigens targeted by the CAR may be directly or indirectly involved in the diseases.

Tumor antigens are proteins that are produced by tumor cells that can elicit an immune response, particularly T-cell mediated immune responses. The selection of the additional targeted antigen of the present disclosure will depend on the particular type of cancer to be treated. Exemplary tumor antigens include, but not limited to, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, CD19, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include: differentiation antigens such as MART-⅟MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, pl5; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO- 1, RCAS 1, SDCCAG16, TA-90\Mac-2 binding protein\cyclophilin C-associated protein, TAAL6, TAG72, TLP, and TPS.

In addition to the one or more antigen binding domain(s) provided herein, the CAR provided herein may further comprise one or more of the following: a linker (e.g., a peptide linker), a transmembrane domain, a hinge region, a signal peptide, an intracellular signaling domain, a co-stimulatory signaling domain, each of which is described in more detail below.

In some embodiments, the CAR contains a signal peptide at the N-terminus of the extracellular domain that directs the nascent receptor into the endoplasmic reticulum, and a hinge peptide at the C-terminus of the extracellular antigen binding domain that makes the receptor more available for binding. The signal peptide can be derived from a molecule selected from the group consisting of CD8α, GM-CSF receptor α, and IgG1 heavy chain. In some embodiments, the signal peptide is derived from CD8α. In some embodiments, the signal peptide comprises an amino acid sequence of SEQ ID NO:67. In some embodiments, the hinge domain is derived from CD8α. In some embodiments, the hinge domain comprises an amino acid sequence of SEQ ID NO:68. The transmembrane domain of the CAR can be derived from a molecule selected from the group consisting of CD8α, CD4, CD28, CD137, CD80, CD86, CD152 and PD1. In some embodiments, the transmembrane domain is derived from CD8α or CD28. In some embodiments, the transmembrane domain comprises an amino acid sequence of SEQ ID NO:69. The intracellular signaling domain of the CAR can comprise a primary intracellular signaling domain and/or a co-stimulatory signaling domain. In some embodiments, the CARs preferably comprises a primary intracellular signaling domain and one or more co-stimulatory signaling domains. The primary intracellular signaling domain may be an immunoreceptor tyrosine-based activation motif (ITAM)-containing domain. The ITAM-containing domain may be CD3-zeta’s cytoplasmic domain having an amino acid sequence of e.g., SEQ ID NO:72, the phosphorylation of which results in T cell activation. The co-stimulatory signaling domain can be derived from a co-stimulatory molecule selected from the group consisting of CD27, CD28, CD137, OX40, CD30, CD40, CD3, LFA-1, ICOS, CD2, CD7, LIGHT, NKG2C, B7-H3, Ligands of CD83 and combinations thereof. In some embodiments, the co-stimulatory signaling domain comprises a cytoplasmic domain of CD28 and/or a cytoplasmic domain of CD137. The cytoplasmic domain of CD28 and the cytoplasmic domain of CD137 comprise amino acid sequences of SEQ ID NO:71 and SEQ ID NO:70, respectively. In some embodiments, the present disclosure provides a Claudin18.2 CAR comprising: from N-terminus to C-terminus, in turn a signal peptide of SEQ ID NO:67, a VHH domain described above for anti-Claudin18.2 VHH selected from the group of SEQ ID NOs: 38-51 and 77-85, a hinge domain of SEQ NO:68, a transmembrane domain of SEQ ID NO:69, a CD137 cytoplasmic domain of SEQ ID NO:70, and a CD3-zeta’s cytoplasmic domain of SEQ ID NO:72. In some embodiments, the Claudin18.2 CAR is monospecific. In other embodiments, the Claudin18.2 CAR is multispecific. In some embodiments, the Claudin18.2 CAR is monovalent. In other embodiments, the Claudin18.2 CAR is multivalent.

### Peptide Linkers

The various single domain antibodies in the multispecific or multivalent CARs described herein may be fused to each other via peptide linkers. In some embodiments, the single domain antibodies are directly fused to each other without any peptide linkers. The peptide linkers connecting different single domain antibodies (e.g., VHH) may be the same or different. Different domains of the CARs may also be fused to each other via peptide linkers.

Each peptide linker in a CAR may have the same or different length and/or sequence depending on the structural and/or functional features of the single domain antibodies and/or the various domains. Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. For example, longer peptide linkers may be selected to ensure that two adjacent domains do not sterically interfere with one another. In some embodiments, a short peptide linker may be disposed between the transmembrane domain and the intracellular signaling domain of a CAR. In some embodiment, a peptide linker comprises flexible residues (such as glycine and serine) so that the adjacent domains are free to move relative to each other. For example, a glycine-serine doublet can be a suitable peptide linker.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G)ₙ, glycine-serine polymers (including, for example, (GS)ₙ, (GSGGS)ₙ, (GGGS)ₙ, and (GGGGS)ₙ, where n is an integer of at least one), glycine-alanine polymers, alanine-serine polymers, and other flexible linkers known in the art. Exemplary peptide linkers are listed in Table 4 below. In a specific embodiment, the peptide linker that connects two or more anti-Claudin18.2 VHH domains provided herein is (GGGGS)ₙ (SEQ ID NO:98), wherein n is optionally 1, 2, 3, 4, 5 or 6.

TABLE 4

Exemplary Peptide Linkers

| | |
|---|---|
| Sequences | SEQ ID NO. |
| GSTSGSGKPGSGEGSTKG | SEQ ID NO:73 |
| TS | SEQ ID NO:74 |
| GGGGSGGGGSGGGGS | SEQ ID NO:75 |
| (GS)ₙ, n is an integer including, e.g., 1, 2, 3, 4, 5, and 6. | SEQ ID NO:94 |
| (GSGGS)ₙ, n is an integer including, e.g., 1, 2, 3, 4, 5, and 6. | SEQ ID NO:95 |
| (GGGS)ₙ, n is an integer including, e.g., 1, 2, 3, 4, 5, and 6. | SEQ ID NO:96 |
| GGGGSGGGGSGGGGGGSGSGGGGSGGGGSGGGGS | SEQ ID NO:97 |
| (GGGGS)ₙ, n is an integer including, e.g., 1, 2, 3, 4, 5, and 6. | SEQ ID NO:98 |
| DGGGS | SEQ ID NO:99 |
| TGEKP | SEQ ID NO: 100 |
| GGRR | SEQ ID NO: 101 |
| GGGGSGGGGSGGGGGGSGSGGGGS | SEQ ID NO: 102 |
| EGKSSGSGSESKVD | SEQ ID NO: 103 |
| KESGSVSSEQLAQFRS | SEQ ID NO: 104 |
| GGRRGGGS | SEQ ID NO: 105 |
| LRQRDGERP | SEQ ID NO: 106 |
| LRQKDGGGSERP | SEQ ID NO: 107 |
| LRQKDGGGSGGGSERP | SEQ ID NO: 108 |
| GSTSGSGKPGSGEGST | SEQ ID NO: 109 |
| GSTSGSGKSSEGKG | SEQ ID NO: 110 |
| KESGSVSSEQLAQFRSLD | SEQ ID NO: 111 |

Other linkers known in the art, for example, as described in WO2016014789, WO2015158671, WO2016102965, US20150299317, WO2018067992, US7741465, Colcher et al., J. Nat. Cancer Inst. 82:1191-1197 (1990), and Bird et al., Science 242:423-426 (1988) may also be included in the CARs provided herein, the disclosure of each of which is incorporated herein by reference.

### 5.3.2. Transmembrane Domain

The CARs of the present disclosure comprise a transmembrane domain that can be directly or indirectly fused to the extracellular antigen binding domain. The transmembrane domain may be derived either from a natural or from a synthetic source. As used herein, a “transmembrane domain” refers to any protein structure that is thermodynamically stable in a cell membrane, preferably an eukaryotic cell membrane. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane- spanning segments and may be further sub-classified based on the number of transmembrane segments and the location of N- and C-termini.

In some embodiments, the transmembrane domain of the CAR described herein is derived from a Type I single-pass membrane protein. In some embodiments, transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD1 la, CD18), ICOS (CD278), 4-1BB (CD137), GITR, CD40, BAFFR, HVEM (LIGHTR), SLAMF7, NKp80 (KLRFl), CD160, CD19, IL-2R beta, IL-2R gamma, IL-7R a, ITGA1, VLA1, CD49a, ITGA4, IA4, CD49D, ITGA6, VLA-6, CD49f, ITGAD, CDl ld, ITGAE, CD 103, ITGAL, CDl la, LFA-1, ITGAM, CDl lb, ITGAX, CDl lc, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain is derived from a molecule selected from the group consisting of CD8α, CD4, CD28, CD137, CD80, CD86, CD152 and PD1.

In some specific embodiments, the transmembrane domain is derived from CD8α. In some embodiments, the transmembrane domain is a transmembrane domain of CD8α comprising the amino acid sequence of SEQ ID NO:69.

Transmembrane domains for use in the CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No.7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the CAR provided herein comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

### 5.3.3. Intracellular Signaling Domain

The CARs of the present disclosure comprise an intracellular signaling domain. The intracellular signaling domain is responsible for activation of at least one of the normal effector functions of the immune effector cell expressing the CARs. The term “effector function” refers to a specialized function of a cell. Effector function of a T cell, for example, may be cytolytic activity or helper activity including the secretion of cytokines. Thus the term “cytoplasmic signaling domain” refers to the portion of a protein which transduces the effector function signal and directs the cell to perform a specialized function. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function signal. The term cytoplasmic signaling domain is thus meant to include any truncated portion of the cytoplasmic signaling domain sufficient to transduce the effector function signal.

In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary cytoplasmic signaling sequences include those derived from CD3ζ, FcR gamma (FCER1G), FcR beta (Fc Epsilon Rib), CD3 gamma, CD3 delta, CD3 epsilon, CD5, CD22, CD79a, CD79b, and CD66d.

In some embodiments, the primary intracellular signaling domain is derived from CD3ζ. In some embodiments, the intracellular signaling domain consists of the cytoplasmic signaling domain of CD3ζ. In some embodiments, the primary intracellular signaling domain of CD3ζ comprises the amino acid sequence of SEQ ID NO:72. In some embodiments, the primary intracellular signaling domain is a cytoplasmic signaling domain of wild-type CD3ζ. In some embodiments, the primary intracellular signaling domain is a functional mutant of the cytoplasmic signaling domain of CD3ζ containing one or more mutations, such as Q65K.

### 5.3.4. Co-Stimulatory Signaling Domain

Many immune effector cells require co-stimulation, in addition to stimulation of an antigen-specific signal, to promote cell proliferation, differentiation and survival, as well as to activate effector functions of the cell. In some embodiments, the CAR comprises at least one co-stimulatory signaling domain. The term “co-stimulatory signaling domain,” as used herein, refers to at least a portion of a protein that mediates signal transduction within a cell to induce an immune response such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD3ζ) and one or more co-stimulatory signaling domains. In some embodiments, the one or more co-stimulatory signaling domains and the primary intracellular signaling domain (such as cytoplasmic signaling domain of CD3ζ) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD3ζ). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CARs described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect). Examples of co-stimulatory signaling domains for use in the CARs can be the cytoplasmic signaling domain of co-stimulatory proteins, including, without limitation, members of the B7/CD28 family (e.g., B7-⅟CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD- 1, PD-L2/B7-DC, and PDCD6); members of the TNF superfamily (e.g.,4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNF SF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, Claudin18.20, CD300a/LMIR1, HLA Class I, HLA- DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-⅟CLEC7A, DPPIV/CD26, EphB6, TIM-⅟KIM-⅟HAVCR, TIM-4, TSLP, TSLP R, lymphocyte function associated antigen-1 (LFA-1), and NKG2C.

In some embodiments, the one or more co-stimulatory signaling domains are selected from the group consisting of CD27, CD28, CD137, OX40, CD30, CD40, CD3, lymphocyte function-associated antigen-1(LFA-1), CD2, CD7, LIGHT, NKG2C, B7-H3 and ligands that specially bind to CD83 (such as CD83 and MD-2).

In some embodiments, the intracellular signaling domain in the CAR of the present disclosure comprises a co-stimulatory signaling domain derived from CD137 (i.e., 4-1BB). In some embodiments, the intracellular signaling domain comprises a cytoplasmic signaling domain of CD3ζ and a co-stimulatory signaling domain of CD137. In some embodiments, the intracellular signaling domain comprises a co-stimulatory signaling domain of CD137 comprising the amino acid sequence of SEQ ID NO:70. In some embodiments, the intracellular signaling domain in the CAR of the present disclosure comprises a co-stimulatory signaling domain derived from CD28 comprising an amino acid sequencwe of SEQ ID NO:71.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

### 5.3.5. Hinge Region

The CARs of the present disclosure may comprise a hinge domain that is located between the extracellular antigen binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is derived from CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α. In some embodiments, the hinge domain of CD8α comprises the amino acid sequence of SEQ ID NO:68.

Hinge domains of antibodies, such as an IgG, IgA, IgM, IgE, or IgD antibodies, are also compatible for use in the pH-dependent chimeric receptor systems described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N- terminus of the transmembrane domain is a peptide linker, such as a (GxS)n linker, wherein x and n, independently can be an integer between 3 and 12, including 3, 4, 5, 6, 7, 8, 9, 10, 11, 12, or more.

### 5.3.6. Signal Peptide

The CARs of the present disclosure may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8α, GM-CSF receptor α, and IgG1 heavy chain. In some embodiments, the signal peptide is derived from CD8α. In some embodiments, the signal peptide of CD8α comprises the amino acid sequence of SEQ ID NO:67.

### 5.3.7. Exemplary CARs

Exemplary CARs are generated as shown in Section 6 below, such as those listed in Table 5 below.

TABLE 5

Amino Acid Sequence ID Numbers of CAR and corresponding VHH

| | | | |
|---|---|---|---|
| CAR Code | CAR SEQ ID NO. | VHH Code | VHH SEQ ID NO. |
| LIC182501 | SEQ ID NO:53 | VHH182501 | SEQ ID NO:38 |
| LIC182502 | SEQ ID NO:54 | VHH182502 | SEQ ID NO:39 |
| LIC182503 | SEQ ID NO:55 | VHH182503 | SEQ ID NO:40 |
| LIC182504 | SEQ ID NO:56 | VHH182504 | SEQ ID NO:41 |
| LIC182505 | SEQ ID NO:57 | VHH182505 | SEQ ID NO:42 |
| LIC182506 | SEQ ID NO:58 | VHH182506 | SEQ ID NO:43 |
| LIC182507 | SEQ ID NO:59 | VHH182507 | SEQ ID NO:44 |
| LIC182508 | SEQ ID NO:60 | VHH182508 | SEQ ID NO:45 |
| LIC182509 | SEQ ID NO:61 | VHH182509 | SEQ ID NO:46 |
| LIC182510 | SEQ ID NO:62 | VHH182510 | SEQ ID NO:47 |
| LIC182511 | SEQ ID NO:63 | VHH182511 | SEQ ID NO:48 |
| LIC182512 | SEQ ID NO:64 | VHH182512 | SEQ ID NO:49 |
| LIC182513 | SEQ ID NO:65 | VHH182513 | SEQ ID NO:50 |
| LIC182514 | SEQ ID NO:66 | VHH182514 | SEQ ID NO:51 |
| LIC182513H4 | SEQ ID NO:86 | VHH182513H4 | SEQ ID NO:78 |
| LIC182513H7 | SEQ ID NO:87 | VHH182513H7 | SEQ ID NO:79 |
| LIC182513H8 | SEQ ID NO:88 | VHH182513H8 | SEQ ID NO:80 |
| LIC182513H9 | SEQ ID NO:89 | VHH182513H9 | SEQ ID NO:81 |
| LIC182513H10 | SEQ ID NO:90 | VHH182513H10 | SEQ ID NO:82 |
| LIC182511H6 | SEQ ID NO:91 | VHH182511H6 | SEQ ID NO:83 |
| LIC182511H8 | SEQ ID NO:92 | VHH182511H8 | SEQ ID NO:84 |
| LIC182511H9 | SEQ ID NO:93 | VHH182511H9 | SEQ ID NO:85 |

In some embodiments, provided herein is a CAR comprising or consisting of the amino acid sequence of SEQ ID NO:53. In some embodiments, provided herein is a CAR comprising or consisting of the amino acid sequence of SEQ ID NO:54. In some embodiments, provided herein is a CAR comprising or consisting of the amino acid sequence of SEQ ID NO:55. In some embodiments, provided herein is a CAR comprising or consisting of the amino acid sequence of SEQ ID NO:56. In some embodiments, provided herein is a CAR comprising or consisting of the amino acid sequence of SEQ ID NO:57. In some embodiments, provided herein is a CAR comprising or consisting of the amino acid sequence of SEQ ID NO:58. In some embodiments, provided herein is a CAR comprising or consisting of the amino acid sequence of SEQ ID NO:59. In some embodiments, provided herein is a CAR comprising or consisting of the amino acid sequence of SEQ ID NO:60. In some embodiments, provided herein is a CAR comprising or consisting of the amino acid sequence of SEQ ID NO:61. In some embodiments, provided herein is a CAR comprising or consisting of the amino acid sequence of SEQ ID NO:62. In some embodiments, provided herein is a CAR comprising or consisting of the amino acid sequence of SEQ ID NO:63. In some embodiments, provided herein is a CAR comprising or consisting of the amino acid sequence of SEQ ID NO:64. In some embodiments, provided herein is a CAR comprising or consisting of the amino acid sequence of SEQ ID NO:65. In some embodiments, provided herein is a CAR comprising or consisting of the amino acid sequence of SEQ ID NO:66. In some embodiments, provided herein is a CAR comprising or consisting of the amino acid sequence of SEQ ID NO:86. In some embodiments, provided herein is a CAR comprising or consisting of the amino acid sequence of SEQ ID NO:87. In some embodiments, provided herein is a CAR comprising or consisting of the amino acid sequence of SEQ ID NO:88. In some embodiments, provided herein is a CAR comprising or consisting of the amino acid sequence of SEQ ID NO:89. In some embodiments, provided herein is a CAR comprising or consisting of the amino acid sequence of SEQ ID NO:90. In some embodiments, provided herein is a CAR comprising or consisting of the amino acid sequence of SEQ ID NO:91. In some embodiments, provided herein is a CAR comprising or consisting of the amino acid sequence of SEQ ID NO:92. In some embodiments, provided herein is a CAR comprising or consisting of the amino acid sequence of SEQ ID NO:93.

In certain embodiments, the CAR provided herein comprises amino acid sequences with certain percent identity relative to any one of the CARs exemplified in the Section 6 below such those in Table 5 above.

In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:53. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:54. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:55. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:56. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:57. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:58. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:59. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:60. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:61. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:62. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:63. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:64. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:65. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:66. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:86. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:87. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:88. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:89. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:90. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:91. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:92. In some embodiments, provided herein is a Claudin18.2 CAR comprising a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:93.

In some embodiments, provided herein is an isolated nucleic acid encoding any of the Claudin18.2 CARs provided herein. More detailed description regarding nucleic acid sequences and vectors are provided below.

### 5.4. Engineered Immune Effector Cells

In yet another aspect, provided herein are host cells (such as immune effector cells) comprising any one of the CARs described herein.

Thus, in some embodiments, provided herein is an engineered immune effector cell (such as T cell) comprising a CAR which comprises a polypeptide comprising: (a) an extracellular antigen binding domain comprising one or more anti-Claudin18.2 sdAb(s); (b) a transmembrane domain; and (c) an intracellular signaling domain, wherein the anti-Claudin18.2 sdAb is an anti-Claudin18.2 sdAb as described in Section 5.2 above, including, e.g., the VHH domains in Table 2 and those having one, two or all three CDRs in any of those VHH domains in Table 2. In some embodiments, the anti-Claudin18.2 sdAb is camelid, chimeric, human, or humanized. In some embodiments, the transmembrane domain is selected from the group consisting of CD8α, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, CD2, CD7, LIGHT, NKG2C, B7-H3, ligands of CD83 (such as CD83 and MD-2) and combinations thereof. In some embodiments, the CAR further comprises a hinge domain (such as a CD8α hinge domain) located between the C-terminus of the extracellular antigen binding domain and the N-terminus of the transmembrane domain. In some embodiments, the CAR further comprises a signal peptide (such as a CD8α signal peptide) located at the N-terminus of the polypeptide In some embodiments, the polypeptide comprises from the N-terminus to the C-terminus: a CD8α signal peptide, the extracellular antigen binding domain, a CD8α hinge domain, a CD8α transmembrane domain, a co-stimulatory signaling domain derived from CD137, and a primary intracellular signaling domain derived from CD3ζ.

In some embodiments, provided herein is an engineered immune effector cell (such as T cell) comprising a CAR which comprises a polypeptide comprising: (a) an extracellular antigen binding domain comprising one or more anti-Claudin18.2 sdAb(s); (b) a transmembrane domain; and (c) an intracellular signaling domain, wherein the anti-Claudin18.2 sdAb comprises the amino acid sequence of any one of SEQ ID NOs:38-51 and 77-85. In some embodiments, provided herein is an engineered immune effector cell (such as T cell) comprising a CAR which comprises a polypeptide comprising: (a) an extracellular antigen binding domain comprising one or more anti-Claudin18.2 sdAb(s); (b) a transmembrane domain; and (c) an intracellular signaling domain, wherein the anti-Claudin18.2 sdAb comprises an amino acid sequence having at least 75%, 80%, 85%, 86%, 87%, 88%, 89%, 90%, 91%, 92%, 93%, 94%, 95%, 96%, 97%, 98%, 99%, or 100% sequence identify to the amino acid sequence of any one of SEQ ID NOs:38-51 and 77-85. In some embodiments, the transmembrane domain is selected from the group consisting of CD8α, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, CD2, CD7, LIGHT, NKG2C, B7-H3, ligands of CD83 (such as CD83 and MD-2) and combinations thereof. In some embodiments, the CAR further comprises a hinge domain (such as a CD8α hinge domain) located between the C-terminus of the extracellular antigen binding domain and the N-terminus of the transmembrane domain. In some embodiments, the CAR further comprises a signal peptide (such as a CD8α signal peptide) located at the N-terminus of the polypeptide. In some embodiments, the polypeptide comprises from the N-terminus to the C-terminus: a CD8α signal peptide, the extracellular antigen binding domain, a CD8α hinge domain, a CD8α transmembrane domain, a co-stimulatory signaling domain derived from CD137, and a primary intracellular signaling domain derived from CD3ζ.

In some embodiments, provided herein is an engineered immune effector cell (such as T cell) comprising a CAR which comprises an amino acid sequence selected from the group consisting of SEQ ID NOs: 53-66 and 86-93. In some embodiments, provided herein is an engineered immune effector cell (such as T cell) comprising a CAR which comprises a polypeptide having at least 75%, 80%, 85%, 86%, 87%, 88%, 89%, 90%, 91%, 92%, 93%, 94%, 95%, 96%, 97%, 98%, 99%, or 100% sequence identity to an amino acid sequence selected from the group consisting of SEQ ID NOs: 53-66 and 86-93.

In other embodiments, there is provided an engineered immune effector cell (such as T cell) comprising a multispecific (such as bispecific or trispecific) or multivalent (such as bivalent or trivalent) chimeric antigen receptor (CAR) comprising a polypeptide comprising: (a) an extracellular antigen binding domain comprising a first single domain antibody (sdAb) specifically binding to Claudin18.2 and one or more additional antigen binding domain(s); (b) a transmembrane domain; and (c) an intracellular signaling domain. In some embodiments, the additional antigen binding domain binds to a different epitope of Claudin18.2. In other embodiments, the additional antigen binding domain binds to a different antigen such as CD22, CD19, CD20, CD33, CD38, BCMA, CS1, ROR1, GPC3, CD123, IL-13R, CD138, c-Met, EGFRvIII, GD-2, NY-ESO-1, MAGE A3, and glycolipid F77. In some embodiments, the first sdAb and/or the additional sdAb is camelid, chimeric, human, or humanized. In some embodiments, the first single domain antibody and the additional single domain antibody are fused to each other via a peptide bond or a peptide linker. In some embodiments, the transmembrane domain is selected from the group consisting of CD8α, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, CD2, CD7, LIGHT, NKG2C, B7-H3, ligands of CD83 (such as CD83 and MD-2) and combinations thereof. In some embodiments, the multispecific CAR further comprises a hinge domain (such as a CD8α hinge domain) located between the C-terminus of the extracellular antigen binding domain and the N-terminus of the transmembrane domain. In some embodiments, the multispecific CAR further comprises a signal peptide (such as a CD8α signal peptide) located at the N-terminus of the polypeptide. In some embodiments, the polypeptide comprises from the N-terminus to the C-terminus: a CD8α signal peptide, the extracellular antigen binding domain, a CD8α hinge domain, a CD8α transmembrane domain, a co-stimulatory signaling domain derived from CD137, and a primary intracellular signaling domain derived from CD3ζ.

In some embodiments, the engineered immune effector cell is a T cell, an NK cell, a peripheral blood mononuclear cell (PBMC), a hematopoietic stem cell, a pluripotent stem cell, or an embryonic stem cell. In some embodiments, the engineered immune effector cell is autologous. In some embodiments, the engineered immune effector cell is allogenic.

Also provided are engineered immune effector cells comprising (or expressing) two or more different CARs. Any two or more of the CARs described herein may be expressed in combination. The CARs may target different antigens, thereby providing synergistic or additive effects. The two or more CARs may be encoded on the same vector or different vectors.

The engineered immune effector cell may further express one or more therapeutic proteins and/or immunomodulators, such as immune checkpoint inhibitors. See, e.g., International Patent Application NOs. PCT/CN2016/073489 and PCT/CN2016/087855, which are incorporated herein by reference in their entirety.

### 5.4.1. Vectors

The present disclosure provides vectors for cloning and expressing any one of the CARs described herein. In some embodiments, the vector is suitable for replication and integration in eukaryotic cells, such as mammalian cells. In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the immunomodulator (such as immune checkpoint inhibitor) coding sequence and/or self-inactivating lentiviral vectors carrying chimeric antigen receptors can be packaged with protocols known in the art. The resulting lentiviral vectors can be used to transduce a mammalian cell (such as primary human T cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector comprises any one of the nucleic acids encoding a CAR described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present disclosure. Promoters may be roughly categorized as constitutive promoters or regulated promoters, such as inducible promoters.

In some embodiments, the nucleic acid encoding the CAR is operably linked to a constitutive promoter. Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1 alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. For example, Michael C. Milone et al compared the efficiencies of CMV, hEF1α, UbiC and PGK to drive chimeric antigen receptor expression in primary human T cells, and concluded that hEF1α promoter not only induced the highest level of transgene expression, but was also optimally maintained in the CD4 and CD8 human T cells (Molecular Therapy, 17(8): 1453-1464 (2009)). In some embodiments, the nucleic acid encoding the CAR is operably linked to a hEF1α promoter.

In some embodiments, the nucleic acid encoding the CAR is operably linked to an inducible promoter. Inducible promoters belong to the category of regulated promoters. The inducible promoter can be induced by one or more conditions, such as a physical condition, microenvironment of the engineered immune effector cell, or the physiological state of the engineered immune effector cell, an inducer (i.e., an inducing agent), or a combination thereof.

In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell.

In some embodiments, the vector also contains a selectable marker gene or a reporter gene to select cells expressing the CAR from the population of host cells transfected through lentiviral vectors. Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

In some embodiments, the vector comprises more than one nucleic acid encoding CARs. In some embodiments, the vector comprises a nucleic acid comprising a first nucleic acid sequence encoding a first CAR and a second nucleic acid sequence encoding a second CAR, wherein the first nucleic acid is operably linked to the second nucleic acid via a third nucleic acid sequence encoding a self-cleaving peptide. In some embodiments, the self-cleaving peptide is selected from the group consisting of T2A, P2A and F2A.

### 5.4.2. Immune Effector Cells

“Immune effector cells” are immune cells that can perform immune effector functions. In some embodiments, the immune effector cells express at least FcyRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils.

In some embodiments, the immune effector cells are T cells. In some embodiments, the T cells are CD4+/CD8-, CD4-/CD8+, CD4+/CD8+, CD4-/CD8-, or combinations thereof. In some embodiments, the T cells produce IL-2, TFN, and/or TNF upon expressing the CAR and binding to the target cells, such as Claudin18.2+ tumor cells. In some embodiments, the CD8+ T cells lyse antigen-specific target cells upon expressing the CAR and binding to the target cells.

In some embodiments, the immune effector cells are NK cells. In other embodiments, the immune effector cells can be established cell lines, for example, NK-92 cells.

In some embodiments, the immune effector cells are differentiated from a stem cell, such as a hematopoietic stem cell, a pluripotent stem cell, an iPS, or an embryonic stem cell.

The engineered immune effector cells are prepared by introducing the CARs into the immune effector cells, such as T cells. In some embodiments, the CAR is introduced to the immune effector cells by transfecting any one of the isolated nucleic acids or any one of the vectors described above. In some embodiments, the CAR is introduced to the immune effector cells by inserting proteins into the cell membrane while passing cells through a microfluidic system, such as CELL SQUEEZE^{®} (see, e.g., U.S. Pat. Application Publication No. 20140287509).

Methods of introducing vectors or isolated nucleic acids into a mammalian cell are known in the art. The vectors described can be transferred into an immune effector cell by physical, chemical, or biological methods.

Physical methods for introducing the vector into an immune effector cell include calcium phosphate precipitation, lipofection, particle bombardment, microinjection, electroporation, and the like. Methods for producing cells comprising vectors and/or exogenous nucleic acids are well-known in the art. See, e.g., Sambrook et al. (2001) Molecular Cloning: A Laboratory Manual, Cold Spring Harbor Laboratory, New York. In some embodiments, the vector is introduced into the cell by electroporation.

Biological methods for introducing the vector into an immune effector cell include the use of DNA and RNA vectors. Viral vectors have become the most widely used method for inserting genes into mammalian, e.g., human cells.

Chemical means for introducing the vector into an immune effector cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, RNA molecules encoding any of the CARs described herein may be prepared by a conventional method (e.g., in vitro transcription) and then introduced into the immune effector cells via known methods such as mRNA electroporation. See, e.g., Rabinovich et al., Human Gene Therapy 17:1027-1035 (2006).

In some embodiments, the transduced or transfected immune effector cell is propagated ex vivo after introduction of the vector or isolated nucleic acid. In some embodiments, the transduced or transfected immune effector cell is cultured to propagate for at least about any of 1 day, 2 days, 3 days, 4 days, 5 days, 6 days, 7 days, 10 days, 12 days, or 14 days. In some embodiments, the transduced or transfected immune effector cell is further evaluated or screened to select the engineered mammalian cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)). Suitable expression systems are well known and may be prepared using known techniques or obtained commercially. Other methods to confirm the presence of the nucleic acid encoding the CARs in the engineered immune effector cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

### 5.4.3. Sources of T Cells

In some embodiments, prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available in the art, may be used. In some embodiments, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. Initial activation steps in the absence of calcium may lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer’s instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, in some embodiments, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, Claudin18.2, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations may result in increased cell yield, cell activation, and cell expansion. Further, use of high cell concentrations may allow more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. In some embodiments, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶ /ml. In some embodiments, the concentration used can be from about 1 × 10⁵/ml to 1 × 10⁶ /ml, and any integer value in between.

In some embodiments, the cells may be incubated on a rotator for varying lengths of time at varying speeds at either 2-10° C., or at room temperature.

T cells for stimulation can also be frozen after a washing step. Without being bound by theory, the freeze and subsequent thaw step may provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% dextran 40 and 5% dextrose, 20% human serum albumin and 7.5% DMSO, or 31.25% plasmalyte-A, 31.25% dextrose 5%, 0.45% NaCl, 10% dextran 40 and 5% dextrose, 20% human serum albumin, and 7.5% DMSO or other suitable cell freezing media containing for example, Hespan and PlasmaLyte A. The cells then are frozen to -80° C. at a rate of 1° per minute and stored in the vapor phase of a liquid nitrogen storage tank. Other methods of controlled freezing may be used as well as uncontrolled freezing immediately at -20° C. or in liquid nitrogen.

In some embodiments, cryopreserved cells are thawed and washed as described herein and allowed to rest for one hour at room temperature prior to activation.

Also contemplated in the present disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment, a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815 (1991); Henderson et al., Immun 73:316-321 (1991); Bierer et al., Curr. Opin. Immun. 5:763-773 (1993)). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH.

In some embodiments, T cells are obtained from a patient directly following treatment In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### 5.4.4. Activation and Expansion of T Cells

In some embodiments, prior to or after genetic modification of the T cells with the CARs described herein, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Pat. Application Publication No. 20060121005.

Generally, T cells can be expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD3 antibody include UCHT1, OKT3, HIT3a (BioLegend, San Diego, US) can be used as can other methods commonly known in the art (Graves J, et al., J.Immunol. 146:2102 (1991); Li B, et al., Immunology 116:487 (2005); Rivollier A, et al., Blood 104:4029 (2004)). Examples of an anti-CD28 antibody include 9.3, B-T3, XR-CD28 (Diaclone, Besancon, France) can be used as can other methods commonly known in the art (Berg et al., Transplant Proc. 30(8):3975-3977 (1998); Haanen et al., J. Exp. Med. 190(9):13191328 (1999); Garland et al., J. Immunol Meth. 227(1-2):53-63 (1999)).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Pat. Application Publication Nos. 20040101519 and 20060034810 for artificial antigen presenting cells (aAPCs) that are contemplated for use in activating and expanding T cells in certain embodiments in the present disclosure.

In some embodiments, the T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment, the cells (for example, 10⁴ to 4×10⁸ T cells) and beads (for example, anti-CD3/CD28 MACSiBead particlesa at a recommended titer of 1:100) are combined in a buffer, preferably PBS (without divalent cations such as, calcium and magnesium). Those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present disclosure. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations may result in increased cell yield, cell activation, and cell expansion. Further, use of high cell concentrations may allow more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In some embodiments, the mixture may be cultured for several hours (about 3 hours) to about 14 days or any hourly integer value in between. In another embodiment, the mixture may be cultured for 21 days. In one embodiment, the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### 5.5. Polynucleotides

In some embodiments, provided herein are polynucleotides comprising polynucleotides encoding a polypeptide (i.e., a Claudin18.2 binding moiety or a Claudin18.2 binding CAR) described herein. The term “polynucleotides that encode a polypeptide” encompasses a polynucleotide which includes only coding sequences for the polypeptide as well as a polynucleotide which includes additional coding and/or non-coding sequences. The polynucleotides of the disclosure can be in the form of RNA or in the form of DNA. DNA includes cDNA, genomic DNA, and synthetic DNA; and can be double-stranded or single-stranded, if single stranded can be the coding strand or non-coding (anti-sense) strand.

In some embodiments, the polynucleotide comprises a polynucleotide (e.g., a nucleotide sequence) encoding a polypeptide comprising an amino acid sequence selected from SEQ ID NOs: 38-51 and 77-85. In other embodiments, the polynucleotide comprises a polynucleotide (e.g., a nucleotide sequence) encoding a polypeptide comprising an amino acid sequence selected from SEQ ID NOs: 53-66 and 86-93.

The present disclosure also provides variants of the polynucleotides described herein, wherein the variant encodes, for example, fragments, analogs, and/or derivatives of a Claudin18.2 binding moiety described herein. In some embodiments, the present disclosure provides a polynucleotide comprising a polynucleotide having a nucleotide sequence that is at least about 80% identical, at least about 85% identical, at least about 90% identical, at least about 95% identical, at least about 96% identical, at least about 97% identical, at least about 98% identical, or at least about 99% identical to a polynucleotide sequence encoding a polypeptide described herein.

As used herein, the phrase “a polynucleotide having a nucleotide sequence that is at least about 95% identical to a polynucleotide sequence” means that the nucleotide sequence of the polynucleotide is almost identical to a reference sequence but with up to five point mutations per each 100 nucleotides. In other words, to obtain a polynucleotide having a nucleotide sequence that is at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′or 3′terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change in amino acid sequence (due to the degeneracy of the genetic code). Polynucleotide variants can be produced for a variety of reasons, for example, to optimize codon expression for a particular host (e.g., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease the expression of the encoded polypeptide. In some embodiments, a polynucleotide variant makes increased expression of the encoded polypeptide as compared to a parental polynucleotide sequence. In some embodiments, a polynucleotide variant contributes decreased expression of the encoded polypeptide as compared to a parental polynucleotide sequence.

In some embodiments, a polynucleotide comprises a polynucleotide having a nucleotide sequence that is at least about 80% identical, at least about 85% identical, at least about 90% identical, at least about 95% identical, at least about 96% identical, at least about 97% identical, at least about 98% identical, or at least about 99% identical to a polynucleotide encoding an amino acid sequence selected from SEQ ID NOs:38-51, 53-66, and 77-93. Also provided is a polynucleotide that comprises a polynucleotide that hybridizes to a polynucleotide encoding an amino acid sequence selected from SEQ ID NOs:38-51, 53-66, and 77-93. In some embodiments, the hybridization is done under conditions of high stringency as is known to those skilled in the art.

In some embodiments, a polynucleotide comprises a coding sequence for a polypeptide (e.g., an antibody) fused in the same reading frame to a polynucleotide which aids in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In some embodiments, a polynucleotide comprises a coding sequence for a polypeptide (e.g., an antibody) fused in the same reading frame to a marker or tag sequence. For example, in some embodiments, a marker sequence is a hexa-histidine tag (HIS-tag) that allows for efficient purification of the polypeptide fused to the marker. In some embodiments, a marker sequence is a hemagglutinin (HA) tag derived from the influenza hemagglutinin protein and suitable for a mammalian host (e.g., COS-7 cells). In some embodiments, the marker sequence is a FLAG™ tag. In some embodiments, a marker may be used in conjunction with other markers or tags.

In some embodiments, a polynucleotide is isolated. In some embodiments, a polynucleotide is substantially pure.

Also provided are vectors comprising the nucleic acid molecules described herein. In an embodiment, the nucleic acid molecules can be incorporated into a recombinant expression vector. The present disclosure provides recombinant expression vectors comprising any of the nucleic acids of the disclosure. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

In an embodiment, the recombinant expression vector of the disclosure can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Examples of plant expression vectors include pBI01, pBI01.2, pBI121, pBI101.3, and pBIN19 (Clontech). Examples of animal expression vectors include pEUK-C1, pMAM, and pMAMneo (Clontech). The recombinant expression vector may be a viral vector, e.g., a retroviral vector, e.g., a gamma retroviral vector.

In an embodiment, the recombinant expression vectors are prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, SV40, 2µ plasmid, λ, bovine papilloma virus, and the like.

The recombinant expression vector may comprise regulatory sequences, such as transcription and translation initiation and termination codons, which are specific to the type of host (e.g., bacterium, plant, fungus, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the described expression vectors include, for instance, neomycin/G418 resistance genes, histidinol x resistance genes, histidinol resistance genes, tetracycline resistance genes, and ampicillin resistance genes.

The recombinant expression vector can comprise a native or normative promoter operably linked to the nucleotide sequence of the disclosure. The selection of promoters, e.g., strong, weak, tissue-specific, inducible and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an RSV promoter, an SV40 promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

Also provided are host cells comprising the nucleic acid molecules described herein. The host cell may be any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, Saos, PC12, SP2/0 (American Type Culture Collection (ATCC), Manassas, VA, CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATCC CRL-TIB-196). Other useful cell lines include those derived from Chinese Hamster Ovary (CHO) cells such as CHO-K1SV (Lonza Biologics, Walkersville, MD), CHO-K1 (ATCC CRL-61) or DG44.

### 5.6. Pharmaceutical Compositions

In one aspect, the present disclosure further provides pharmaceutical compositions comprising a single domain antibody, a binding molecule or therapeutic molecule comprising a single domain antibody, or an engineered immune effector cell of the present disclosure. In some embodiments, a pharmaceutical composition comprises a therapeutically effective amount of the single domain antibody, the binding molecule or therapeutic molecule comprising the single domain antibody, or the engineered immune effector cell of the present disclosure and a pharmaceutically acceptable excipient.

In some embodiments, provided herein is a pharmaceutical composition comprising a therapeutically effective amount of the single domain antibody provided herein and a pharmaceutically acceptable excipient.

In some embodiments, provided herein is a pharmaceutical composition comprising a therapeutically effective amount of the therapeutic molecule (such as a fusion protein, immunoconjugate, and a multispecific binding molecule) comprising the single domain antibody provided herein and a pharmaceutically acceptable excipient.

In other embodiments, provided herein is a pharmaceutical composition comprising a therapeutically effective amount of CAR comprising the single domain antibody provided herein and a pharmaceutically acceptable excipient.

In other embodiments, provided herein is a pharmaceutical composition comprising a therapeutically effective amount of engineered immune effector cells provided herein and a pharmaceutically acceptable excipient.

In other embodiments, provided herein is a pharmaceutical composition comprising a therapeutically effective amount of a nucleic acid provided herein, e.g., in a vector, and a pharmaceutically acceptable excipient, e.g., suitable for gene therapy.

In a specific embodiment, the term “excipient” can also refer to a diluent, adjuvant (e.g., Freunds’ adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington’s Pharmaceutical Sciences (1990) Mack Publishing Co., Easton, PA. Such compositions will contain a prophylactically or therapeutically effective amount of the active ingredient provided herein, such as in purified form, together with a suitable amount of excipient so as to provide the form for proper administration to the patient. The formulation should suit the mode of administration.

In some embodiments, the choice of excipient is determined in part by the particular cell, binding molecule, and/or antibody, and/or by the method of administration. Accordingly, there are a variety of suitable formulations.

Typically, acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers may be used to control the pH in a range which optimizes the therapeutic effectiveness, especially if stability is pH dependent. Suitable buffering agents for use with the present disclosure include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives may be added to retard microbial growth. Suitable preservatives for use with the present disclosure include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intramolecular interactions. Exemplary tonicity agents include polyhydric sugar alcohols, trihydric or higher sugar alcohols, such as glycerin, erythritol, arabitol, xylitol, sorbitol and mannitol.

Additional exemplary excipients include: (1) bulking agents, (2) solubility enhancers, (3) stabilizers and (4) agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be present to help solubilize the therapeutic agent as well as to protect the therapeutic protein against agitation-induced aggregation, which also permits the formulation to be exposed to shear surface stress without causing denaturation of the active therapeutic protein or antibody. Suitable non-ionic surfactants include, e.g., polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they are preferably sterile. The pharmaceutical composition may be rendered sterile by filtration through sterile filtration membranes. The pharmaceutical compositions herein generally can be placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

In another embodiment, a pharmaceutical composition can be provided as a controlled release or sustained release system. In one embodiment, a pump may be used to achieve controlled or sustained release (see, e.g., Sefton, Crit. Ref. Biomed. Eng. 14:201-40 (1987); Buchwald et al., Surgery 88:507-16 (1980); and Saudek et al., N. Engl. J. Med. 321:569-74 (1989)). In another embodiment, polymeric materials can be used to achieve controlled or sustained release of a prophylactic or therapeutic agent (e.g., a fusion protein as described herein) or a composition provided herein (see, e.g., Medical Applications of Controlled Release (Langer and Wise eds., 1974); Controlled Drug Bioavailability. Drug Product Design and Performance (Smolen and Ball eds., 1984); Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71:105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, polyethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., Radiotherapy & Oncology 39:179-89 (1996); Song et al., PDA J. of Pharma. Sci. & Tech. 50:372-97 (1995); Cleek et al., Pro. Int′l. Symp. Control. Rel. Bioact. Mater. 24:853-54 (1997); and Lam et al., Proc. Int′l. Symp. Control Rel. Bioact. Mater. 24:759-60 (1997)).

The pharmaceutical compositions described herein may also contain more than one active compound or agent as necessary for the particular indication being treated. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington’s Pharmaceutical Sciences 18th edition.

Various compositions and delivery systems are known and can be used with the therapeutic agents provided herein, including, but not limited to, encapsulation in liposomes, microparticles, microcapsules, recombinant cells capable of expressing the single domain antibody or therapeutic molecule provided herein, construction of a nucleic acid as part of a retroviral or other vector, etc.

In some embodiments, the pharmaceutical composition provided herein contains the binding molecules and/or cells in amounts effective to treat or prevent the disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about ninety-nine percent of active ingredient, preferably from about 0.1% to about 70%, most preferably from about 1% to about 30% of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. Alternatively, antibody can be administered as a sustained release formulation, in which case less frequent administration is required.

For administration of the composition, the dosage may range from about 0.0001 to 100 mg/kg, and more usually 0.01 to 5 mg/kg, of the host body weight. For example, dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an anti-Claudin18.2 antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 µg/mL and in some methods about 25-300 µg/mL. The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid See, e.g., Sustained and Controlled Release Drug Delivery Systems, J. R. Robinson, ed., Marcel Dekker, Inc., New York, 1978.

In some embodiments, wherein the pharmaceutical composition comprises any one of the engineered immune cells described herein, the pharmaceutical composition is administered at a dosage of at least about any of 10⁴, 10⁵, 10⁶, 10⁷, 10⁸, or 10⁹ cells/kg of body weight of the individual. In some embodiments, the pharmaceutical composition is administered at a dosage of any of about 10⁴ to about 10⁵, about 10⁵ to about 10⁶, about 10⁶ to about 10⁷, about 10⁷ to about10⁸, about 10⁸to about 10⁹, about 10⁴ to about 10⁹, about 10⁴ to about 10⁶, about 10⁶ to about 10⁸, or about 10⁵ to about 10⁷ cells/kg of body weight of the individual. In some embodiments, the pharmaceutical composition is administered at a dose of at least about any 1×10⁵, 2×10⁵ 3×10^{5,} 4×10^{5,} 5×10⁵, 6×10⁵ 7×10^{5,} 8×10^{5,} 9×10^{5,} 1×10⁶ 2×10⁶, 3×10⁶, 4×10⁶, 5×10⁶, 6×10⁶, 7×10⁶, 8×10⁶, 9×10⁶, 1×10⁷ cells/kg or more. In some embodiments, the pharmaceutical composition is administered at a dose of about 3×10⁵ to about 7×10⁶ cells/kg, or about 3 ×10⁶ cells/kg.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

### 5.7. Methods and Uses

In another aspect, provided herein are methods for using and uses of the Claudin18.2 binding molecules provided herein, including the anti-Claudin18.2 VHHs, chimeric antigen receptors (CARs), and/or engineered cells expressing the recombinant receptors.

### 5.7.1. Therapeutic Methods and Uses

The present disclosure also provides methods of using or use of the Claudin18.2 binding moiety, the multispecific or multivalent molecule, the conjugate, the oncolytic virus, the CAR, the engineered immune cell, the polynucleotide encoding the same, the recombinant expression vector comprising the polynucleotide, the cell containing the expression vectors, or the pharmaceutical composition disclosed herein in treating Claudin18.2-expressing cancer or tumor. Without being bound by theory, the Claudin18.2 binding moiety, the multispecific or multivalent molecule, the conjugate, the oncolytic virus, the CAR, and the engineered immune cell may specifically target Claudin18.2-expressing cancer cells in vivo, thereby exerting their therapeutic effect of eliminating, lysing and/or killing cancer cells.

In some embodiments, provided herein is a method of treating a Claudin18.2-expressing tumor or cancer in a subject in need thereof, comprising administering to the subject a therapeutically effective amount of the Claudin18.2 binding moiety, the multispecific or multivalent molecule, the conjugate, the oncolytic virus, the CAR, the engineered immune cell, the polynucleotide encoding the same, the recombinant expression vector comprising the polynucleotide, the cell containing the expression vectors, or the pharmaceutical composition disclosed herein. In some embodiments, the Claudin18.2-expressing cancers or tumors that can be treated are solid or non-solid tumors. As a non-limiting example, the Claudin18.2-expressing cancer or tumor is gastric, esophageal, gastro-esophageal, liver, lung, colorectal, endometrial, breast, pancreatic, testicular, cervical, ovarian, or glioma cancer or tumor.

In some embodiments, the Claudin18.2-expressing cancer or tumor is gastric cancer or tumor. In some embodiments, the Claudin18.2-expressing cancer or tumor is a primary gastric adenocarcinoma. In some embodiments, the Claudin18.2-expressing cancer or tumor is esophageal cancer or tumor. In some embodiments, the Claudin18.2-expressing cancer or tumor is gastro-esophageal cancer or tumor. In some embodiments, the Claudin18.2-expressing cancer or tumor is any cancer or tumor involving Claudin18.2 expression. In some embodiments, the Claudin18.2-expressing cancer or tumor is any cancer or tumor involving ectopic activation of Claudin18.2 (e.g., pancreatic, esophageal, ovarian, and lung tumors). In some embodiments, a Claudin18.2-expressing cancer or tumor is a primary cancer or tumor (e.g., gastric tumor). In some embodiments, a Claudin18.2-expressing cancer or tumor is the metastases of a primary cancer or tumor. As a non-limiting example, in some embodiments, the Claudin18.2-expressing cancer or tumor is lymph node metastases or distant metastases of gastric cancer adenocarcinomas. In some embodiments, the Claudin18.2-expressing cancer or tumor is located at the ovary (e.g., Krukenberg tumors). In certain embodiments, the Claudin18.2-expressing cancer or tumor is correlated with a histological subtype. As non-limiting examples, in some embodiments, Claudin18.2-expressing cancer or tumor is adenocarcinoma (but not squamous cell cancer) of the esophagus, a mucinous (but not serous) ovarian cancer, or a ductal pancreatic adenocarcinoma (but not pancreatic islet cancer).

In some embodiments, the methods disclosed herein can decrease the number of Claudin18.2 positive tumor cells in a subject. In some embodiments, the methods disclosed herein can decrease tumor burden in a subject. In some embodiments, a Claudin18.2 binding moiety disclosed herein can be used to harness a subject’s natural defense mechanisms including CDC and ADCC to eliminate malignant or cancer cells.

Methods for monitoring patient response to administration of a pharmaceutical composition disclosed herein are known in the art and can be employed in accordance with methods disclosed herein. In some embodiments, methods known in the art can be employed to monitor the patient’s response to administration of a pharmaceutical composition disclosed herein. In some embodiments, methods known in the art can be used to monitor lesion sizes, and/or lymph node sizes.

As a non-limiting example, in some embodiments, contrast-enhanced CT scans may be used to detect and/or monitor lesions and/or lymph nodes in a patient. In some embodiments, administration of a pharmaceutical composition disclosed herein can reduce the size of lesions detected by CT scans in a patient. In some embodiments, administration of a pharmaceutical composition disclosed herein can cause shrinkage of abnormal lymph nodes.

In certain embodiments, the methods provided herein can be used to treat cancers or reduce tumor burden in a subject, wherein the cancer or tumor is Claudin18.2-expressing cancer or tumor. In one embodiment, the methods provided herein are used to treat cancer. It is understood that a method of treating cancer may have anti-tumor effects that ameliorates a sign or symptom associated with cancer. Such signs or symptoms include, but are not limited to, reducing tumor burden, including inhibiting tumor growth, slowing tumor growth rate, reducing tumor size, reducing tumor cell number, eliminating a tumor, all of which can be measured using routine tumor imaging techniques well known in the art. Other signs or symptoms associated with cancer include, but are not limited to, fatigue, pain, weight loss, and other signs or symptoms associated with various cancers. In one non-limiting example, the methods provided herein can reduce tumor burden. Thus, administration of the pharmaceutical compositions of the invention can reduce tumor cell number, reduce tumor size, and/or eradicate the tumor in a subject. The tumor can be a solid tumor. The methods of the invention can also provide for increased or lengthened survival of a subject having a cancer. Additionally, methods of the invention can provide for an increased immune response in a subject against the cancer.

In the methods of the invention, a therapeutically effective amount of the Claudin18.2 binding moiety (e.g., antibodies), or the Claudin18.2 binding moiety (e.g. antibody) containing biopharmaceutical (e.g., CAR-T cells) is administered to a subject in need of cancer treatment. The subject can be a mammal. In some embodiments, the subject is human. The administration is to elicit an anti-cancer response, palliating a subject’s condition. Eliminating cancer or tumor cells in a subject may occur, but any clinical improvement constitutes a benefit. Clinical improvement comprises cancer or tumor size reduction and decreased progression rate.

The method of the disclosure can be used to treat a subject who has a history of cancer, and is responsive to a prior therapy. The prior therapy may be a surgical resection, radiotherapy, or traditional chemotherapy. The subject may have no clinically measurable tumor but is at a risk of disease progression, either near the original tumor site, or by metastases. Such a subject may be further subdivided into the high-risk or low-risk group, depending on features observed before or after the initial treatment, which is known in the art. High-risk subject is one having tumor invaded neighboring tissues, or getting lymph nodes involved. Optionally, the biopharmaceuticals or compositions of the disclosure may be administered to prevent the occurrence of cancer in a subject susceptible to cancers, for example, according to family history and/or genetic testing.

The subject receiving the administration may have an advanced form of disease, and the treatment is to suppress mitigation or reversal of disease progression. The subject cured by other methods before may use the present treatment to decrease or delay the risk of recurrence. Additionally, refractory or recurrent malignancies can be treated using the pharmaceutical compositions disclosed herein.

An amount of the biopharmaceuticals or compositions effective for producing the desired effect may be administered to a subject for cancer or tumor treatment. An effective amount or therapeutically effective amount is an amount sufficient to provide a beneficial or desired clinical result upon treatment. An effective amount can be provided in a single administration or a series of administrations (one or more doses). An effective amount can be provided in a bolus or by continuous perfusion. In terms of treatment, an effective amount is an amount that is sufficient to palliate, ameliorate, stabilize, reverse or slow the progression of the disease, or otherwise reduce the pathological consequences of the disease. The effective amount can be determined by the physician for a particular subject. Several factors are typically taken into account when determining an appropriate dosage to achieve an effective amount. These factors include age, sex and weight of the subject, the condition being treated, the severity of the condition and the form and effective concentration of the biopharmaceuticals of the disclosure being administered.

Combination therapy using agents with different mechanisms of action may result in additive or synergetic effects. Combination therapy can allow for a lower dose of each agent than is used in monotherapy, thereby reducing toxic side effects and/or increasing the therapeutic index of the agent disclosed herein. Combination therapy can decrease the likelihood that resistant cancer cells will develop.

Thus, the present disclosure also provides methods of combination therapy in which the therapeutic agents provided herein such as Claudin18.2 binding moieties or CAR-T cells of the disclosure are co-administered with one or more additional agents that are effective in inhibiting tumor growth in a subject. In one embodiment, the invention provides a method for inhibiting tumor growth in a subject comprising administering to the subject Claudin18.2 binding moieties or CAR-T cells of the disclosure with one or more additional antibodies, such as an anti-OX40 antibody, an anti-TIM-3 antibody, an anti-CD137 antibody, an anti-GITR antibody, an anti-LAG-3 antibody, an anti-PD-L1 antibody, and anti-PD-1 antibody and/or an anti-CTLA-4 antibody.

The additional therapy can be administered prior to, concurrently with, or subsequent to administration of the biopharmaceuticals or pharmaceutical compositions described herein. Combined administration may include co-administration, either in a single pharmaceutical formulation or using separate formulations, or consecutive administrations in either order but generally within a time period such that all active agents can exert their biological activities simultaneously. A person skilled in the art can readily determine appropriate regimens for administering a Claudin18.2 binding moiety or a related biopharmaceutical described herein and an additional therapy in combination, including the timing and dosing of an additional agent to be used in a combination therapy, based on the needs of the subject being treated.

In some specific embodiments, provided herein is a method for treating a disease or disorder in a subject comprising administering to the subject a binding molecule comprising a single domain antibody that binds to Claudin18.2 as described in Section 5.2 above, including, e.g., those with CDRs in Table 2, those comprising the amino acid sequence of any one of SEQ ID NOs:38-51 and 77-85, and those comprising an amino acid sequence having at least 75%, 80%, 85%, 90%, 91%, 92%, 93%, 94%, 95%, 96%, 97%, 98%, or 99% sequence identify to any one of SEQ ID NOs:38-51 and 77-85. In some embodiments, the disease or disorder is a Claudin18.2 associated disease or disorder. In some embodiments, the disease or disorder is a Claudin18.2-expressing tumor or cancer. In some embodiments, the Claudin18.2-expressing cancers or tumors are solid or non-solid tumors. In some embodiments, the Claudin18.2-expressing cancer or tumor is gastric cancer or tumor. In some embodiments, the Claudin18.2-expressing cancer or tumor is a primary gastric adenocarcinoma. In some embodiments, the Claudin18.2-expressing cancer or tumor is esophageal cancer or tumor. In some embodiments, the Claudin18.2-expressing cancer or tumor is gastro-esophageal cancer or tumor. In some embodiments, the Claudin18.2-expressing cancer or tumor is any cancer or tumor involving Claudin18.2 expression. In some embodiments, the Claudin18.2-expressing cancer or tumor is any cancer or tumor involving ectopic activation of Claudin18.2 (e.g., pancreatic, esophageal, ovarian, and lung tumors). In some embodiments, a Claudin18.2-expressing cancer or tumor is a primary cancer or tumor (e.g., gastric tumor). In some embodiments, a Claudin18.2-expressing cancer or tumor is the metastases of a primary cancer or tumor. As a non-limiting example, in some embodiments, the Claudin18.2-expressing cancer or tumor is lymph node metastases or distant metastases of gastric cancer adenocarcinomas. In some embodiments, the Claudin18.2-expressing cancer or tumor is located at the ovary (e.g., Krukenberg tumors). In certain embodiments, the Claudin18.2-expressing cancer or tumor is correlated with a histological subtype. As non-limiting examples, in some embodiments, Claudin18.2-expressing cancer or tumor is adenocarcinoma (but not squamous cell cancer) of the esophagus, a mucinous (but not serous) ovarian cancer, or a ductal pancreatic adenocarcinoma (but not pancreatic islet cancer).

In other embodiments, provided herein is a method for treating a disease or disorder comprising administering to the subject an engineered immune effector cell (such as T cell) as provided in Section 5.4, including, e.g., the cells comprising a CAR provided in Section 5.3. In some embodiments, the engineered immune cell administered to the subject comprises a CAR comprising a polypeptide comprising: (a) an extracellular antigen binding domain comprising one or more anti-Claudin18.2 sdAb(s); (b) a transmembrane domain; and (c) an intracellular signaling domain, wherein the anti-Claudin18.2 sdAb is as described in Section 5.2 above, including e.g., those with CDRs in Table 2, those comprising the amino acid sequence of any one of SEQ ID NOs:38-51 and 77-85, and those comprising an amino acid sequence having at least 75%, 80%, 85%, 90%, 91%, 92%, 93%, 94%, 95%, 96%, 97%, 98%, or 99% sequence identify to SEQ ID NOs:38-51 and 77-85. In some embodiments, the engineered immune cell administered to the subject comprises a CAR comprising an amino acid sequence selected from the group consisting of SEQ ID NOs: 53-66 and 86-93, or comprising a polypeptide having at least 75%, 80%, 85%, 86%, 87%, 88%, 89%, 90%, 91%, 92%, 93%, 94%, 95%, 96%, 97%, 98%, or 99% sequence identity to an amino acid sequence selected from the group consisting of SEQ ID NOs:53-66 and 86-93. In some embodiments, the disease or disorder is a Claudin18.2 associated disease or disorder. In some embodiments, the disease or disorder is a Claudin18.2-expressing tumor or cancer. In some embodiments, the Claudin18.2-expressing cancers or tumors are solid or non-solid tumors. In some embodiments, the Claudin18.2-expressing cancer or tumor is gastric cancer or tumor. In some embodiments, the Claudin18.2-expressing cancer or tumor is a primary gastric adenocarcinoma. In some embodiments, the Claudin18.2-expressing cancer or tumor is esophageal cancer or tumor. In some embodiments, the Claudin18.2-expressing cancer or tumor is gastro-esophageal cancer or tumor. In some embodiments, the Claudin18.2-expressing cancer or tumor is any cancer or tumor involving Claudin18.2 expression. In some embodiments, the Claudin18.2-expressing cancer or tumor is any cancer or tumor involving ectopic activation of Claudin18.2 (e.g., pancreatic, esophageal, ovarian, and lung tumors). In some embodiments, a Claudin18.2-expressing cancer or tumor is a primary cancer or tumor (e.g., gastric tumor). In some embodiments, a Claudin18.2-expressing cancer or tumor is the metastases of a primary cancer or tumor. As a non-limiting example, in some embodiments, the Claudin18.2-expressing cancer or tumor is lymph node metastases or distant metastases of gastric cancer adenocarcinomas. In some embodiments, the Claudin18.2-expressing cancer or tumor is located at the ovary (e.g., Krukenberg tumors). In certain embodiments, the Claudin18.2-expressing cancer or tumor is correlated with a histological subtype. As non-limiting examples, in some embodiments, Claudin18.2-expressing cancer or tumor is adenocarcinoma (but not squamous cell cancer) of the esophagus, a mucinous (but not serous) ovarian cancer, or a ductal pancreatic adenocarcinoma (but not pancreatic islet cancer).

### 5.7.2. Diagnostic and Detection Methods and Uses

In another aspect, provided herein are methods involving use of the binding molecules provided herein, e.g., VHHs that binds Claudin18.2 and molecules (such as conjugates and complexes) containing such VHHs, for detection, prognosis, diagnosis, staging, determining binding of a particular treatment to one or more tissues or cell types, and/or informing treatment decisions in a subject, such as by the detection of Claudin18.2 and/or the presence of an epitope thereof recognized by the antibody.

In some embodiments, an anti-Claudin18.2 antibody (such as any one of the anti-Claudin18.2 single domain antibodies described herein) for use in a method of diagnosis or detection is provided. In a further aspect, a method of detecting the presence of Claudin18.2 in a biological sample is provided. In certain embodiments, the method comprises detecting the presence of Claudin18.2 protein in a biological sample. In certain embodiments, Claudin18.2 is human Claudin18.2. In some embodiments, the methods are diagnostic and/or prognostic methods in association with a Claudin18.2-expressing disease or disorder. The methods in some embodiments include incubating and/or probing a biological sample with the antibody and/or administering the antibody to a subject. In certain embodiments, a biological sample includes a cell or tissue or portion thereof, such as tumor or cancer tissue or biopsy or section thereof. In certain embodiments, the contacting is under conditions permissive for binding of the anti-Claudin18.2 antibody to Claudin18.2 present in the sample. In some embodiments, the methods further include detecting whether a complex is formed between the anti-Claudin18.2 antibody and Claudin18.2 in the sample, such as detecting the presence or absence or level of such binding. Such a method may be an in vitro or in vivo method. In one embodiment, an anti-Claudin18.2 antibody is used to select subjects eligible for therapy with an anti-Claudin18.2 antibody or engineered antigen receptor, e.g., where Claudin18.2 is a biomarker for selection of patients.

In some embodiments, a sample, such as a cell, tissue sample, lysate, composition, or other sample derived therefrom is contacted with the anti-Claudin18.2 antibody and binding or formation of a complex between the antibody and the sample (e.g., Claudin18.2 in the sample) is determined or detected. When binding in the test sample is demonstrated or detected as compared to a reference cell of the same tissue type, it may indicate the presence of an associated disease or disorder, and/or that a therapeutic containing the antibody will specifically bind to a tissue or cell that is the same as or is of the same type as the tissue or cell or other biological material from which the sample is derived. In some embodiments, the sample is from human tissues and may be from diseased and/or normal tissue, e.g., from a subject having the disease or disorder to be treated and/or from a subject of the same species as such subject but that does not have the disease or disorder to be treated. In some cases, the normal tissue or cell is from a subject having the disease or disorder to be treated but is not itself a diseased cell or tissue, such as a normal tissue from the same or a different organ than a cancer that is present in a given subject.

Various methods known in the art for detecting specific antibody-antigen binding can be used. Exemplary immunoassays include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be used so as to meet the needs of various uses of the method which are often dictated by the availability of assay equipment and compatible immunoassay procedures. Exemplary labels include radionuclides (e.g. ¹²⁵I, ¹³¹I, ³⁵S, ³H, or ³²P and/or chromium (⁵¹Cr), cobalt (⁵⁷Co), fluorine (¹⁸F), gadolinium (¹⁵³Gd, ¹⁵⁹Gd), germanium (⁶⁸Ge), holmium (¹⁶⁶Ho), indium (¹¹⁵In, ¹¹³In, ¹¹²In, ¹¹¹In), iodine (¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (186Re, 188Re), rhodium (105Rh), rutheroium (97Ru), samarium (¹⁵³Sm), scandium (⁴⁷Sc), selenium (⁷⁵Se), (⁸⁵Sr), sulphur (³⁵S), technetium (⁹⁹Tc), thallium (²⁰¹Ti) tin (¹¹³Sn, ¹¹⁷Sn), tritium (3H), xenon (¹³³Xe), ytterbium (¹⁶⁹Yb, ¹⁷⁵Yb), yttrium (⁹⁰Y),), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). Various general techniques to be used in performing the various immunoassays noted above are known.

In certain embodiments, labeled antibodies (such as anti-Claudin18.2 single domain antibodies) are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. In other embodiments, antibodies are not labeled, and the presence thereof can be detected using a labeled antibody which binds to any of the antibodies.

### 5.8. Kits and Articles of Manufacture

Further provided are kits, unit dosages, and articles of manufacture comprising any of the single domain antibodies, the chimeric antigen receptors, or the engineered immune effector cells described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer’s solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

For the sake of conciseness, certain abbreviations are used herein. One example is the single letter abbreviation to represent amino acid residues. The amino acids and their corresponding three letter and single letter abbreviations are as follows:

| | | | | | |
|---|---|---|---|---|---|
| Amino acid | Three letter | One letter | Amino acid | Three letter | One letter |
| alanine | Ala | (A) | leucine | Leu | (L) |
| arginine | Arg | (R) | lysine | Lys | (K) |
| asparagine | Asn | (N) | methionine | Met | (M) |
| aspartic acid | Asp | (D) | phenylalanine | Phe | (F) |
| cysteine | Cys | (C) | proline | Pro | (P) |
| glutamic acid | Glu | (E) | serine | Ser | (S) |
| glutamine | Gln | (Q) | threonine | Thr | (T) |
| glycine | Gly | (G) | tryptophan | Trp | (W) |
| histidine | His | (H) | tyrosine | Tyr | (Y) |
| isoleucine | Ile | (I) | valine | Val | (V) |

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### 6. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 6.1. Example 1—Generation of Anti-Claudin18.2 Antibodies

To develop antibodies containing binding moieties with high binding affinity to Claudin18.2, camels were immunized and a phage-display library was constructed to identify anti-Claudin18.2 VHH leads.

### Cell Lines Construction

Dubca.huCLDN18.2.Luc cell line was developed in house following the method briefly described as below. Human CLDN18.2 coding sequence (NM_001002026.2) was synthesized and subcloned to pLVX-puro (Clontech, Cat. No. 632164) between EcoRI and BamHI restriction sites to obtain the transfer vector pLVX-huCLDN18.2.Luc.Puro. Lentivirus were packaged by transient transfection of Lenti-X 293T host cells with a mix of plasmids including psPAX2, pMD.2G and pLVX-huCLDN18.2.Luc.Puro. 0.5×10⁶ Dubca cells (ATCC® CRL2276™) were transduced with 100 µL obtained LV-huCLDN18.2.Luc.PuroR lentivirus. And the transduced cells were selected with Puromycin to obtain the Dubca.huCLDN18.2.Luc cells by refreshing selection culture medium (Eagle’s Minimum Essential Medium supplemented with 10% FBS and 2 µg/mL puromycin) every 2-3 days. After 3 rounds of selection, the obtained cell clones were harvested by trypsinization. The obtained cells were well preserved and ready for further use.

The expression of human CLDN18.2 on the Dubca.huCLDN18.2.Luc cell line was validated by flow cytometry using a mouse anti-human Claudin18.2 antibody from GenScript. Briefly, 2.0×10⁵ Dubca.huCLDN18.2.Luc cells or Dubca cells were incubated with anti-human Claudin18.2 primary antibody at 4° C. for 30 min, followed by wash-centrifugation-supernatant depletion-cell washing cycles for 3 times with DPBS. After wash, cell pellets were re-suspended with DPBS and incubated with a secondary antibody (PE anti-human IgG Fc Antibody, Biolegend, Cat. No. 409304) at 4° C. for 30 min in dark. The cells were then treated by wash-centrifugation-supernatant depletion-cell washing cycles for 3 times with DPBS. The cells were then run on Attune NXT flow cytometry (Thermo Fisher) to detect the expression level of human Claudin18.2. The mean fluorescence intensity (MFI) of labeled Dubca.huCLDN18.2 was 641.59 folds higher than that of Dubca cells.

Several other cell lines were also developed to be expressing either human CLDN18.1 (NM_016369.3) or human CLDN18.2 protein (NM_001002026.2) according to the Dubca.huCLDN18.2.Luc cell line generation and preparation procedures described above. Such cell lines included gastric cancer cell lines, including KATOIII (ATCC#HTB-103) and NUGC4 (JCRB0834), a pancreatic cancer cell line PANC1 (ATCC#CRL-1469™) and HEK293T (Clontech, Cat. No. 632180). The host cells were transduced with the in-house prepared lentivirus LV-huCLDN18.1.Luc.Puro or LV-huCLDN18.2.Luc.Puro stock. And the transduced cells were selected with Puromycin to obtain the stable cells. Briefly, KATOIII is a human gastric cancer cell line, which can express low levels of human Claudin 18.2. KATOIII.huCLDN18.2.Luc cell line was developed to be co-expressing human Claudin 18.2 and firefly luciferase linked by a 2A peptide. KATOIII. huCLDN18.1.Luc cell line was developed to be co-expressing human Claudin 18.1 and firefly luciferase linked by a 2A peptide. KATOIII.Luc cell line was developed to be over-expressing firefly luciferase alone. PANC1 is a pancreatic cancer cell line, which does not express either human Claudin18.1 or human Claudin18.2 protein. Cell lines expressing human Claudin 18.1 (NM_016369.3) or human Claudin 18.2 (NM_001002026.2) was developed based on PANC1 cells and named as PANC1.huCLDN18.1.Luc and PANC1.huCLDN18.2.Luc, respectively. Similarly, NUGC4.Luc cell line was developed to be expressing Luciferase reporter. HEK293T.huCLDN18.1.Luc and HEK293T.huCLDN18.2 .Luc stable cells were developed to be co-expressing human Claudin 18.1 and Luciferase, and co-expressing human Claudin18.2 and Luciferase, respectively.

### Animal Immunization and Immune Response Assay

Immunogen comprising Dubca.huCLDN18.2 cells as prepared above was mixed with adjuvant or PBS and injected to adult male doublet camels. The animals were immunized for five times, typically with or without CFA (Complete Freund’s Adjuvant) each time at about 1-week to 2-week intervals. Peripheral blood samples were collected at the pre-immunization stage and after each immunization. Lymphocytes were isolated by gradient centrifugation from about 100 mL of peripheral blood, and supplemented with RNALater™ and stored at -80° C. Sera were obtained by centrifugation of anti-coagulated blood samples and stored at -80° C.

After multiple rounds of immunization, the titer of antigen-specific antibodies was measured by HEK293T.CLDN18.2.Luc cell binding, and the data suggested that the antibody titer increased significantly with immunization.

### VHH Phage Display Library Construction

Total RNAs were extracted from the isolated lymphocytes using TRIZOL® Reagent (Thermofisher, Cat. No. 15596026) according to the manufacturer’s instruction, and reverse transcribed into cDNAs with an oligo(dT)₂₀ primer using PrimeScript™ 1st Strand cDNA Synthesis Kit (Takara, Cat. No. 6110A) according to the manufacturer’s protocol. Forward and reverse specific degenerate primers (see Chinese patent CN105555310B) were designed to amplify the VHH fragments, which had two SfiI restriction sites introduced. The VHH fragments were amplified using a two-step polymerase chain reaction (PCR), and the PCR products were digested with SfiI and gel purified, and then inserted into phagemid vector pFL249 (see CN105555310B), which were electro-transferred into E.coli cells to generate the phage display VHH immune library.

A small portion of the transformed cells were diluted and streaked on 2×YT plates supplemented with 100 µg/mL ampicillin. The colonies were counted to calculate the library size. Positive clones were randomly picked and sequenced to assess the quality of the library. The rest of the transformed cells were streaked onto 245-mm YT plates supplemented with 100 µg/mL ampicillin and 2% glucose. Lawns of colonies were scraped off the plates. A small aliquot of the cells was used for library plasmid isolation. The rest was supplemented with glycerol and stored at -80° C. as stock.

### Phage Display Panning

Two rounds of panning were performed as described below. KATOIII.huCLDN18.2.Luc cells, HEK293T.huCLDN18.2.Luc cells and NUGC4.huCLDN18.2.Luc cells prepared above were incubated with the phage library, respectively. After extensive washing, bound phage particles were eluted and collected by using 1 mg/mL trypsin. In a second round selection, the phage particles as obtained were incubated with KATOIII.CLDN18.2.Luc cells, and bound phages were eluted and collected using the 1 mg/mL trypsin.

After the panning, bound phage particles were eluted and collected using 100 mM trimethylamine (TEA), and the eluate was neutralized by 1 M Tris-HCl (pH 7.4). Half of the eluate was then used to infect exponentially growing E. coli TG1 cells (OD600 = 0.4~0.6) for output titration.

Phage ELISA was performed to identify clones specific to the target antigens. Individual output phage clones were grown in 96-deep-well plates and rescued by M13KO7 helper phage overnight. To identify clones that bound to antigen proteins, 96-well ELISA microtiter plates were coated with recombinant human HEK293T.huCLDN18.1.Luc and HEK293T.huCLDN18.2.Luc cells respectively in coating buffer overnight at 4° C., and the plates were then blocked with blocking buffer. After blocking, approximately 50 µL per well of phage supernatant from overnight cell culture was added to the plates for 1.5-hour incubation at 4° C. The plates were washed four times, and the HRP-conjugated anti-M13 monoclonal antibody was added to the plates for 45-minute incubation at 4° C. The plates were again washed five times and substrate solution was added to the wells for color developing. Absorbance at 450 nm was measured for each well.

To further identify clones that bind HEK293T.huCLDN18.2.Luc cells, the HEK293T.CLDN18.2.Luc cells were blocked with blocking buffer at room temperature for 1 hour. After blocking, approximately 20 µL per well of phage supernatant from overnight cell culture was added to the cell solutions for 1-hour incubation at room temperature. After the cells were washed 4 times, the HRP-conjugated anti-M13 monoclonal antibody was added for 30 min incubation at room temperature. The cells were washed five times and substrate solution was then added for developing. The absorption was measured at 450 nm.

After screening, clones with high binding capacities were selected and sequenced. The CDR and VHH sequences were summarized in Table 2.

### 6.2. Example 2-Preparation of Chimeric Anti-Claudin18.2 Antibodies

The VHH coding sequences for the selected antibodies were optimized for human codon biased expression with GenScript OptimumGene™- Codon Optimization, synthesized and fused to human IgG1Fc coding sequence (SEQ ID NO:76) for transient expression in chimeric formats. The chimeric antibody coding sequence were cloned into pcDNA3.4-based mammalian expression system plasmids and the plasmids were maxi-prepared for protein production by GenScript cataloged services. A plasmid was constructed to express a control antibody 175DX-hIgG1Fc containing a single chain variable fragment (SEQ ID NO:52) with IMAB362′s heavy chain variable region and light chain variable region fused to human IgG1Fc. IMAB362 (Claudiximab, Zolbetuximab), a chimeric monoclonal IgG1 antibody, has been studied in numerous clinical trials for the treatment of patients with advanced gastroesophageal cancers (Sahin et al., Journal of Hematology & Oncology, 10: 105 (2017)).

The chimeric antibodies were expressed in Expi293F cells (Thermofisher, Cat# A14527) transfected with chimeric VHH-hIgG1Fc or 175DX-hIgG1Fc coding plasmids. Briefly, one day before transfection, Expi293FTM cells were seeded at an appropriate density in Erlenmeyer Flasks (Corning) with serum-free Expi293TM Expression Medium (Thermo Fisher Scientific) and grown at 37° C. with 8% CO₂ on an orbital shaker (VWR Scientific). On the day of transfection, chimeric antibody coding sequence containing plasmids and ExpiFectamine™ 293 Reagent were mixed at an optimal ratio and then added into the flask with cells ready for transfection. Approximately 16-18 hours post-transfection, ExpiFectamine™ 293 Transfection Enhancer 1 and ExpiFectamine™ 293 Transfection Enhancer 2 were added to each flask.

On Day 6, cell culture broth was centrifuged and followed by filtration. The obtained cell culture supernatant was loaded onto an affinity purification column at an appropriate flowrate. After washing and elution with appropriate buffers, the eluted fractions were pooled and buffer exchanged to the final formulation buffer. The purified protein was analyzed by SDS-PAGE and western blot to determine the molecular weight and purity. The concentration was determined by A280 method. Exemplary CARs and the corresponding sequences are shown in Table 5.

### 6.3. Example 3—Characterization of Chimeric Anti-Claudin 18.2 Antibodies

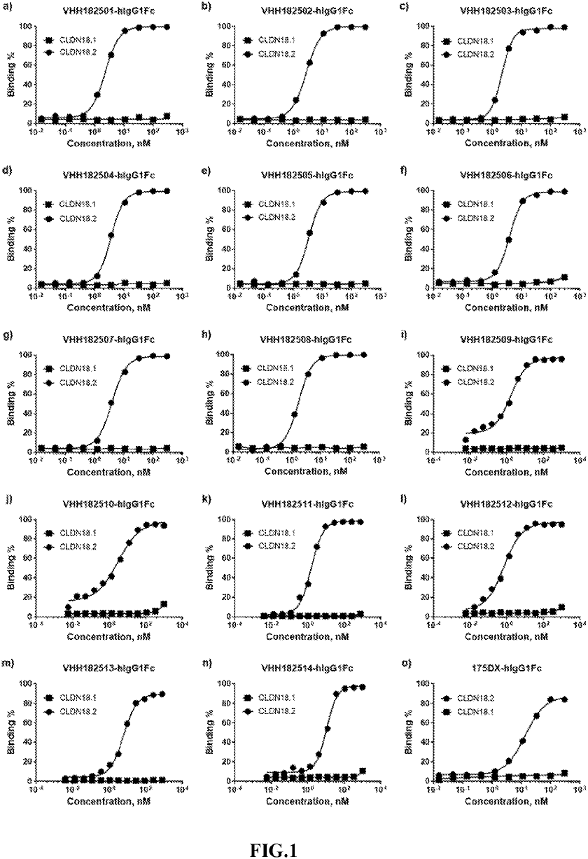

The anti-Claudin 18.2 antibodies were tested for binding capacities to PANC1.huCLDN18.1.Luc and PANC1.huCLDN18.2.Luc cells by cell based flow cytometry. Briefly, 5.0×10⁵ PANC1.huCLDN18.1.Luc or PANC1.huCLDN18.2.Luc cells in DPBS (pH7.2) were incubated with serially diluted anti-Claudin18.2 VHH-hIgG1Fc at 4° C. for 45 min, followed by wash-centrifugation-supernatant depletion-cell washing cycles for 3 times with DPBS. After wash, cell pellets were re-suspended in DPBS and incubated with a secondary antibody (1:200, Alexa Fluor™ 488 GOAT anti-human IgG (H+L) Antibody, Invitrogen, Cat. No. A1 1013) at 4° C. for 30 min in dark. The cells were then subject to wash-centrifugation-supernatant depletion-cell washing cycles for 3 times with DPBS. The cells were then run on Attune NXT flow cytometry to detect antibody-PANC1.huCLDN18.1.Luc or PANC1.huCLDN18.2.Luc binding level. Binding capabilities of each test antibody were described as Percentage of Binding, calculated with (cells with antibody binding/total cells)× 100 %. 175DX-hIgG1Fc as prepared in Example 2 was used as a benchmark in the assay. The results were shown in FIG. 1 and Table 6.

TABLE 6

Binding capacity of chimeric antibodies to PANC1.huCLDN18.1.Luc or PANCI.huCLDN18.2.Luc cells

| Antibody ID | EC₅₀, nmol/L | |
|---|---|---|
| | Binding on PANC1.huCLDN18.1.Luc | Binding on PANC1.huCLDN18.2.Luc |
| VHH182501-hIgG1Fc | n.s. | 2.33 |
| VHH182502-hIgG1Fc | n.s. | 2.82 |
| VHH182503-hIgG1Fc | n.s. | 2.16 |
| VHH182504-hIgG1Fc | n.s. | 3.65 |
| VHH182505-hIgG1Fc | n.s. | 3.45 |
| VHH182506-hIgG1Fc | n.s. | 3.92 |
| VHH182507-hIgG1Fc | n.s. | 3.96 |
| VHH182508-hIgG1Fc | n.s. | 1.70 |
| VHH182509-hIgG1Fc | n.s. | 1.67 |
| VHH182510-hIgG1Fc | n.s. | 3.24 |
| VHH182511-hIgG1Fc | n.s. | 1.61 |
| VHH182512-hIgG1Fc | n.s. | 0.71 |
| VHH182513-hIgG1Fc | n.s. | 6.13 |
| VHH182514-hIgG1Fc | n.s. | 10.92 |
| 175DX-hIgG1Fc | n.s. | 13.51 |

“n.s.” indicates no significant binding.

As shown in FIG. 1 and Table 6, the chimeric antibodies of the disclosure showed potent binding to PANC1.huCLDN18.2.Luc in a dose dependent manner, but not to PANC1.huCLDN18.1.Luc cells, suggesting their binding specificity to human CLDN18.2. The chimeric antibodies had approximately 1.24 to 19.03 folds higher binding potencies (EC₅₀ values ranging from 0.71 nM to 10.92 nM) to PANCI.huCLDN18.2.Luc cells than that of the benchmark 175DX-hIgG1Fc (EC₅₀ = 13.51 nM). In addition, the maximum binding% for each chimeric antibody of the disclosure was almost 100% at the concentration of 10¹ nM, while the binding% of the benchmark was about 40% at the same concentration.

### 6.4. Example 4-Preparation of Engineered T Cells Containing Chimeric Antigen Receptors (CARs)

A nucleotide acid molecule encoding a chimeric antigen receptor (CAR) backbone polypeptide comprising from the N-terminus to the C-terminus a CD8α hinge domain (SEQ ID NO:68), a CD8α transmembrane domain (SEQ ID NO:69), a CD137 co-stimulatory signaling domain (SEQ ID NO:70), and a CD3ζ intracellular signaling domain (SEQ ID NO:72) were synthesized and cloned into a pre-modified lentiviral vector (pLSINK-BBzBB) downstream and operably linked to a constitutive hEF1α promoter, or cloned into a cloning vector (PT7-0985) downstream and linked to a T7 promoter for in vitro transcription. Multi-cloning sites (MCS) in the vector allowed insertion of a nucleic acid sequence comprising a Kozak sequence operably linked to a nucleic acid sequence encoding a CD8α signal peptide (SEQ ID NO:67) fused to the N-terminus of an anti-Claudin18.2 VHH fragment into the CAR backbone vector, upstream and operably linked to the CAR backbone sequence. The nucleic acid sequence encoding the CD8α signal peptide and the anti-Claudin18.2 VHH fragment was chemically synthesized and cloned into the PT7-0985 via the MluI (5′- ACGCGT-3′) and SpeI (5′-ACTAGT-3′) or pLSINK-BBzBB CAR backbone via the EcoRI (5′-GAATTC-3′) and SpeI (5′-ACTAGT-3′) restriction sites by molecular cloning techniques known in the art.

The CAR construct RNAs were prepared by in vitro transcription using mMESSAGE mMACHINE T7 Kit and & Poly(A) Tailing Kit (Thermo Fisher AM1344 and AM1350). Briefly, the purified plasmids were proceeded to in vitro transcription reactions and incubation according to the instructions of the Kit. The transcribed RNAs (IVT-RNA) were then purified using RNeasy Mini kit (QIAGEN, Cat#75144). Finally, the IVT-RNAs were liquated at 10 µL/vial, stored at -80° C. immediately or used directly for CAR-T preparation.

The lentivirus packaging plasmid mixture containing pMDLg.pRRE (Addgene#12251), pRSV-REV (Addgene#12253) and pMD2.G (Addgene#12259) were pre-mixed with the vectors expressing CAR constructs at a pre-optimized ratio with polyetherimide (PEI), then incubated at 25° C. for 5 min. Then HEK293 cells were added into the transfection mix. Afterwards, cells were incubated overnight in a cell incubator with 5% CO₂ at 37° C. The supernatants were collected after centrifuged at 4° C. and 3000 g for 15 min, and filtered through a 0.45 µm PES filter followed by ultra-centrifugation for lentivirus concentration. Then the supernatants were carefully discarded and the virus pellets were rinsed cautiously with pre-chilled DPBS. The viruses were liquated properly, and stored at -80° C. The virus titer was determined by a titration method via transduction of CHO (Chinese hamster ovarian) cell line.

Leukocytes were collected from healthy donors by apheresis, and cell concentration was adjusted to 5×10⁶ cells/mL in TexMACS GMP Medium&1L (Miltenyi #170-076-309). Leukocytes were then mixed with 0.9% NaCl solution at 1:1 (v/v) ratio. 3 mL lymphoprep medium was added to a 15 mL centrifuge tube, and 6 mL of diluted lymphocyte mix was slowly layered on top of the lymphoprep medium. The lymphocyte mix was centrifuged at 800 g for 30 min without brakes at 20° C. Lymphocyte buffy coat was then collected with a 200 µL pipette. The harvested fraction was diluted at least 6 folds with 0.9% NaCl or R10 to reduce density of the solution. The harvested fraction was then centrifuged at 250 g for 10 minutes at 20° C. The supernatant was aspirated completely, and 10 mL of R10 was added to the cell pellet to resuspend the cell pellet. The mixture was further centrifuged at 250 g for 10 min at 20° C. The supernatant was again aspirated. 2 mL of 37° C. pre-warmed TexMACS GMP Medium&1L (Miltenyi #170-076-309) with 300 IU/mL IL-2 was added to the cell pellet, and the cell pellet was resuspended softly. The cell number was determined following Trypan Blue staining, and this PBMC sample was ready for later experiments.

Human T cells were purified from PBMCs using Miltenyi Pan T cell isolation kit (Cat#130-096-535), following manufacturer’s protocols as described below. Cell number was first determined and the cell suspension was centrifuged at 300 g for 10 min. The supernatant was then aspirated completely, and the cell pellets were re-suspended in 40 µL MACS buffer (DPBS supplemented with 8 µM EDTA + 0.5%FBS) per 10⁷ total cells. 10 µL of Pan T Cell Biotin-Antibody Cocktail was added per 10⁷ total cells, mixed thoroughly and incubated for about 5 min in the refrigerator (2~8° C.). 30 µL of MACS buffer was then added per 10⁷ cells. 20 µL of Pan T Cell MicroBead Cocktail was added per 10⁷ cells. The cell suspension mixture was mixed well and incubated for additional 10 min in the refrigerator (2~8° C.). A minimum of 500 µL was required for magnetic separation. For magnetic separation, an LS column was placed in the magnetic field of a suitable MACS Separator. The column was prepared by rinsing with 3 mL of buffer. The cell suspension was then applied onto the column, and flow-through containing the unlabeled cells was collected, which represented the enriched T cell fractions. Additional T cells were collected by washing the column with 3 mL of buffer and collecting unlabeled cells that passed through. These unlabeled cells again represented the enriched T cells, and were combined with the flow-through from previous step. The pooled enriched T cells were then centrifuged and re-suspended in TexMACS GMP Medium&1L (Miltenyi #170-076-309)+300 IU/mL IL-2.

The prepared T cells were subsequently pre-activated for 48-96 hours with human T Cell TransAct™ (Miltenyi #130-111-160) according to manufacturer’s protocol in which anti-CD3/CD28 MACSiBead particles were added at a bead-to-cell ratio of 1:2.

The pre-activated T cells were transduced with lentivirus stock at multiplicity of infection (MOI) of 10, by adding lentivirus stock directly to the culture medium (TexMACS GMP Medium). After 48 hours, the transduced cells were then transferred to a cell culture incubator for transgene expression in cell culture incubator with 5% CO₂ at 37° C.

Alternatively, the pre-activated T cells were electroporated with CAR IVT-RNAs, with protocol as described below. Pre-activated T cells were harvested by centrifugation at 300 g for 10 min at room temperature. After completely removing supernatant, cell pellets were resuspended in Celetrix 103 buffer, and cell concentration was assessed by trypan blue staining and aliquoted at 4~6 million human T cell per 120 µL. The electroporation mix was prepard by adding 10 µg CAR-mRNA to each aliquots of preactivated T cells. Electroporation was then performed at a pre-optimized voltage and pulse (820 V/20ms) by using Celetrix electroporation apparatus. Immediately after the electroporation process, cells were transferred to a new pre-heated medium, and cultured overnight at a humidified 37° C. with 5 % CO₂ incubator until analysis.

On day 2 to day 4 post IVT-RNA electroporation, electroporated T cells were harvested. CAR expression levels were assessed by flow cytometry. Briefly, 1×10⁶ electroporated T cells were collected from each group, then incubated with FITC labeled MonoRab™ Rabbit Anti-Camelid VHH Antibody (iFluor 647) mAb (Genscript Cat#A01994) for VHH-based CAR-T, for 30 min at 4° C. Upon completion of incubation, cells were harvested and washed with DPBS, then centrifuged at 300 g for 10 min at 20° C. UnT represented T cells un-transduced with CARs.

The expression level of the prepared CAR-T cells were read on Attune NxT Flow Cytometer (Thermo Fisher), and data were summarized in Table 7.

TABLE 7

Expression level of CAR-T cells

| Cells code | CAR+% | Cells code | CAR+% |
|---|---|---|---|
| LIC182501 CAR-T | 95.0% | LIC182511 CAR-T | 94.4% |
| LIC182502 CAR-T | 97.1% | LIC182512 CAR-T | 97.7% |
| LIC182503 CAR-T | 95.7% | LIC182513 CAR-T | 94.5% |
| LIC182504 CAR-T | 91.6% | LIC182514 CAR-T | 97.4% |
| LIC 182505 CAR-T | 94.8% | UnT | 1.1% |
| LIC182506 CAR-T | 97.2% | | |
| LIC182507 CAR-T | 94.7% | | |
| LIC182508 CAR-T | 96.9% | | |
| LIC182509 CAR-T | 98.2% | | |
| LIC182510 CAR-T | 60.4% | | |
| UnT | 0.9% | | |

### 6.5. Example 5—Characterization of Engineered CAR-T Cells

Cytotoxicity assay was performed. CAR-T cells prepared in Example 4 were co-incubated with PANC1.huCLDN18.2.Luc, PANC1.huCLDN18.1.Luc, NUGC4.Luc, and KATOIII.Luc cells prepared in Example 1, respectively, at 10:1 or 2:1 effector (CAR-T cell) to target cell ratio (E:T) for 20-24 hours. To assay the cytotoxicity of CAR-T cells on tumor cells, One-glo luminescent luciferase assay reagents (Promega#E6120) were prepared according to manufacturer’s protocol, and added to the co-cultured cells to detect the remaining luciferase activity in the wells. The remaining luciferase activity directly correlated to the number of viable target cells in the well. The specific cytotoxicity was calculated by the formula: Specific Cytotoxicity %=100%×(1-(RLUₛₐₘₚₗₑ-RLUₘᵢₙ)/(RLU_{UnT}-RLUₘᵢₙ)). RLUₛₐₘₚₗₑ represented for the luciferase activity as measured in the well with CAR-T cells having anti-Claudin18.2 CARs of the disclosure. RLUₘᵢₙ referred to the luciferase activity as determined in the well added with Triton X-100 at a final concentration of 1% when the cytotoxicity assay was initiated, and RLU_{UnT} referred to the luciferase activity as determined in the well with T cells un-transduced with CARs.

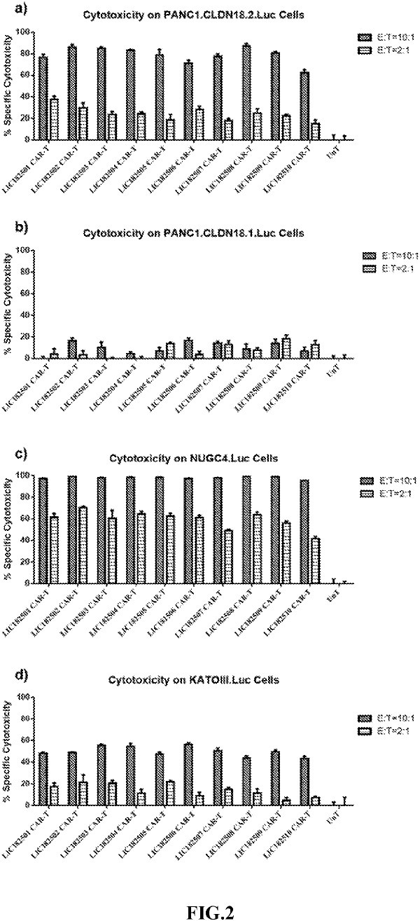

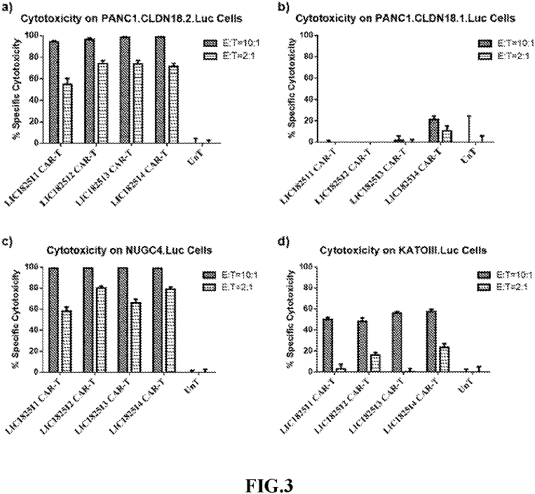

As shown in FIG. 2 (parts a and b) and FIG. 3 (parts a and b), the LIC182501 CAR-T cells to LIC182514 CAR-T cells showed potent killing effects to PANC1.CLDN18.2.Luc cells which stably over-expressed human Claudin18.2 (approximate 98.5% human CLDN18.2 expression level as determined by flowcytometry), while these CAR-T cells did not show significant cytotoxicity on PANC1.CLDN18.1.Luc cells which stably over-expressed human Claudin18.1. LIC182501 CAR-T cells to LIC182514 CAR-T cells showed high cytotoxicity on PANC1.CLDN18.2.Luc cells at the E/T ratio of 10:1. At a lower E/T ratio of 2:1, LIC182501 CAR-T cells (37.443% ± 2.951%) showed higher cytotoxicity on PANC1.CLDN18.2.Luc cells than other VHH-based CAR-T of the disclosure.

KATOIII.Luc cells has low CLDN18.2 expression level. The capability of killing cells expressing low antigen (CLDN18.2) levels may provide more clinical benefits in terms of allowing more patients to be involved in the treatment. As shown in FIG. 2 (part d), at the E/T ratio of 10:1, LIC182501 CAR-T cells to LIC182510 CAR-T cells (from 43.26% ± 2.35% to 56.22% ± 1.58%) showed good killing potencies on KATOIII.Luc cell. At the lower E/T ratio at 2:1, LIC182501 CAR-T cells to LIC182510 CAR-T cells still showed potent killing potencies on KATOIII.Luc cells (from 4.59% ± 2.84% to 21.85% ±12.4%). As shown in FIG. 3 (part d), at E/T ratio of 10:1, LIC182513- LIC182514 CAR-T cells showed slightly higher killing potencies than LIC182511 CAR-T cells and LIC182512 CAR-T cells. At E/T ratio of 2:1, LIC182512 CAR-T cells and LIC182514 CAR-T cells showed stronger cytotoxicity than LIC182511 CAR-T cells and LIC182513 CAR-T cells.

As also shown in FIG. 2 (part c) and FIG. 3 (part c), all of the tested CAR-T cells were potent in killing the NUGC4.Luc cells which was approximate 67.1% human CLDN18.2 protein expression as determined by flow cytometry. LIC182501 CAR-T cells to LIC182510 CAR-T cells (from 95.06% ± 0.43% to 99.17% ± 0.12%) showed high killing potencies on NUGC4 cells at the E/T ratio of 10:1. At a lower E/T ratio of 2:1, LIC182501 CAR-T cells to LIC182509 CAR-T cells (from 48.90% ± 1.13% to 74.18% ± 2.78%) also showed higher killing potencies on NUGC4 cells than LIC182510 CAR-T cells. LIC182511 to LIC182514 CAR-T cells showed comparable cytotoxicity potencies at E/T ratio of 10:1. LIC182512 CAR-T cells and LIC182514 CAR-T cells show a little higher cytotoxicity potencies at E/T ratio of 2:1.

### 6.6. Example 6-In Vivo Anti-Tumor Efficacy Study of Claudin18.2 CAR-T Cells

In vivo anti-tumor efficacy of Claudin18.2 CAR-T cells were evaluated in NUGC4.Luc gastric cancer systemic NCG (NOD-Prkdcem26Cd52Il2rgem26Cd22/NjuCrl) mouse model.

CAR-T cells were prepared using lentiviral transduction as described above.

Immune deficient mice NCG were subcutaneously injected with 3 × 10⁶ NUGC4.Luc cells per mouse. 12 days later, the NCG mice with subcutaneous xenograft tumors were injected CLDN18.2 specific CAR-T cells or CD19 specific CAR-T cells (1×10⁶/mouse) by intravenous route. CD19 specific CAR-T (CD19 CAR-T) were CAR-T cells targeting human CD19 antigen, comprising an scFv from FMC63 (NCBI access number # ADM64594.1) and CAR framework identical to the other CAR tested (CD8α signal peptide-antigen binding domain-CD8α hinge and transmembrane domain-CD137 co-stimulatory signaling domain-CD3ζ intracellular signaling domain). These mice were monitored for tumor volume to evaluate the antitumor efficacy for approximately 4 weeks after administration of T cells. One-way ANNOVA was used for multiple comparisons performed by GraphPad prism 6.

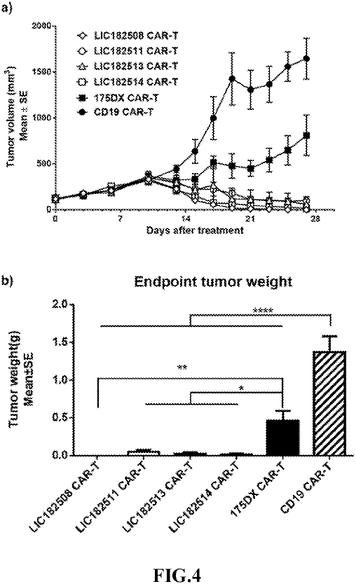

As shown in FIG. 4 (part a), Claudin18.2 CAR-T cells were potent to show anti-tumor effects on the NUGC4 cell engrafted xenograft model in vivo. As compared to CD19 CAR-T, Claudin18.2 CAR-T were potent to control or eliminate tumor growth in this gastric cancer xenograft model. As shown in FIG. 4 (part a) and Table 8, as compared to the benchmark CAR-T (175DX CAR-T based on IMAB362), VHH based CAR-T candidates including LIC182508, LIC182511, LIC182513 and LIC182514 CAR-T cells showed significantly stronger anti-tumor effects in vivo.

TABLE 8

Significance of anti-tumor efficacy difference by days after CAR-T treatment

| | | | |
|---|---|---|---|
| Groups / Days after CAR-T treatment | Day 23 | Day 25 | Day 27 |
| 175DX CAR-T vs. LIC182508 CAR-T | ** | *** | **** |
| 175DX CAR-T vs. LIC182511 CAR-T | ** | *** | **** |
| 175DX CAR-T vs. LIC182513 CAR-T | ** | *** | **** |
| 175DX CAR-T vs. LIC182514 CAR-T | * | ** | *** |

(****:p<0.0001; ***: 0.0001<p<0.001; **: 0.001<p<0.01; *: 0.01<p<0.05)

At the end of the in vivo study, the tumor tissues were collected and weighted to evaluate the anti-tumor efficacy of tested CAR-T candidates. As shown in FIG. 4 (part b), weight of tumors collected from LIC182508, LIC182511, LIC182513, LIC182514 and 175DX CAR-T rescuded mice were significantly lower than that from mice treated with CD19 CAR-T. By comparing with 175DX CAR-T, VHH based CAR-T cells were significantly more capable to reduce or eliminate the gastric cancer cells in vivo.

In sum, the data demonstrated that CLDN18.2 specific CAR-T were potent to inhibit gastric cancer growth in vivo. And the tested VHH based Claudin18.2 CAR-T were plausible to show significantly stronger anti-tumor potency than 175DX CAR-T.

### 6.7. Example 7—Generation and Characterization of Humanized Chimeric Anti-Claudin18.2 Antibodies

To humanize antibodies containing binding moieties with high binding affinity to Claudin18.2, anti-Claudin18.2 VHH antibody amino acid residues were humanized according to the description by Ce’cile Vincke et al (J. Biol. Chem. 2009, 284:3273-3284) or by the method of resurfacing framework of VHH antibodies.

Universal humanized VHH framework h-NbBcII10FGLA (Protein Data Bank (PDB) code: 3EAK) designed by Cecile Vincke et al., was adopted for humanization design based on sequence homology. According to the canonical structures and residue substitution preferences, multiple sites on the engrafted humanized sequences were recovered from camelid VHH182513 or VHH182511 sequences.

The homologous modeling of camelid VHH182513 and VHH182511 were performed using the modeling software MODELLER. The reference homologous sequence of VHH182513 was a Zn-bound camelid single domain antibody (PDB code: 6KSN) and camelid VHH HL6 antibody (PDB code: 1OP9) for VHH182511. According to alignment with human germline gene, IGHV3-30*01 was chosen as human acceptor for VHH182513 and IGHV3-23*05 was chosen as human acceptor for VHH182511. Relative solvent accessibility of the amino acids is calculated according to the three-dimensional structure of the protein. If one of the amino acids of VHH is exposed to a solvent, it was replaced with the original amino acid. The sequence ID number of humanized anti-Claudin18.2 VHHs and their parental VHHs are listed in Table 10.

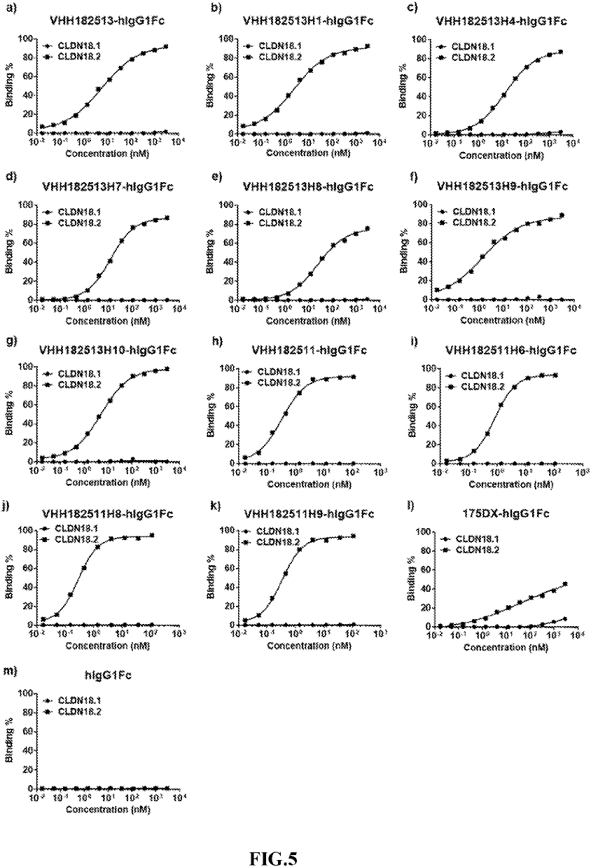

Coding sequence of selected clones of humanized single domain antibody were codon optimized for human codon biased expression with GenScript OptimumGene™-Codon Optimization. The resulting coding DNA fragments were synthesized and fused to nucleotides nucleotide coding human IgG1 Fc portion (SEQ ID NO:76), for transient expression in chimeric formats. The constructs were cloned into individual pcDNA3.4-based plasmids downstream of a synthesized signal peptide (SEQ ID NO:112: MGWSCIILFLVATATGVHS) for secretory expression. The humanized chimeric anti-Claudin18.2 antibodies were obtained using the mothed described in Example 2. The binding capacity of humanized chimeric antibodies to PANC1.huCLDN18.1.Luc or PANC1.huCLDN18.2.Luc cells were measured. The results are shown in FIG. 5 and Table 9.

TABLE 9

Binding capacity of chimeric antibodies to PANC1.huCLDN18.1.Luc or PANCI.huCLDN18.2.Luc cells

| Antibody ID | EC₅₀ (nM) | |
|---|---|---|
| | Binding on PANC1.huCLDN18.1.Luc | Binding on PANC1.huCLDN18.2.Luc |
| VHH182513-hIgG1Fc | n.s. | 5.731 |
| VHH182513H1-hIgG1Fc | n.s. | 2.378 |
| VHH182513H4-hIgG1Fc | n.s. | 14.12 |
| VHH182513H7-hIgG1Fc | n.s. | 12.65 |
| VHH182513H8-hIgG1Fc | n.s. | 25.45 |
| VHH182513H9-hIgG1Fc | n.s. | 1.423 |
| VHH182513H10-hIgG1Fc | n.s. | 5.028 |
| VHH182511-hIgG1Fc | n.s. | 0.328 |
| VHH182511H6-hIgG1Fc | n.s. | 0.8301 |
| VHH182511H8-hIgG1Fc | n.s. | 0.2862 |
| VHH182511H9-hIgG1Fc | n.s. | 0.3406 |
| 175DX-hIgG1Fc | low | 49.22 |
| hIgG1Fc | n.s. | n.s. |

“n.s.” indicates no significant binding; “low” indicates low binding and not saturated to calculate EC₅₀ value.

Chimeric antibodies which were successfully expressed and obtained with reasonable protein yield were tested of their binding to huCLDN18.2 versus huCLDN18.1. As shown in FIG. 5 and Table 9, the chimeric humanized or non-humanized VHH antibodies showed potent binding to PANC1.huCLDN18.2.Luc in a dose dependent manner, but not to PANC1.huCLDN18.1.Luc cells, suggesting their binding specificity to huCLDN18.2. These chimeric antibodies had approximately 1.93 to 171.98 folds higher binding potencies (EC₅₀ values ranging from 0.2862 nM to 25.45 nM) to PANC1.huCLDN18.2.Luc cells than that of the benchmark 175DX-hIgG1Fc (EC₅₀=49.22 nM).

### 6.8. Example 8—Preparation and Characterization of Humanized Claudin18.2 CAR-T Cells

The CAR backbone described in Example 4 was used and the process of preparing CARs described in Example 4 was followed. CAR constructs with humanized anti-Claudin18.2 VHHs, including, LIC182513H4, LIC182513H7, LIC182513H8, LIC182513H9, LIC182513H10, LIC182511H6, LIC182511H8, and LIC182511H9 were obtained (Table 10). The sequence ID number of parental CAR, namely LIC182513 and LIC182511 are also shown in Table 10.

TABLE 10

Amino Acid Sequences of CARs and corresponding VHH domains

| | | | |
|---|---|---|---|
| CAR Code | AA SEQ ID NO. | VHH Code | AA SEQ ID NO. |
| LIC182513 | SEQ ID NO:65 | VHH182513 | SEQ ID NO:50 |
| LIC182513H4 | SEQ ID NO:86 | VHH182513H4 | SEQ ID NO:78 |
| LIC182513H7 | SEQ ID NO:87 | VHH182513H7 | SEQ ID NO:79 |
| LIC182513H8 | SEQ ID NO:88 | VHH182513H8 | SEQ ID NO:80 |
| LIC182513H9 | SEQ ID NO:89 | VHH182513H9 | SEQ ID NO:81 |
| LIC182513H10 | SEQ ID NO:90 | VHH182513H10 | SEQ ID NO:82 |
| LIC182511 | SEQ ID NO:63 | VHH182511 | SEQ ID NO:48 |
| LIC182511H6 | SEQ ID NO:91 | VHH182511H6 | SEQ ID NO:83 |
| LIC182511H8 | SEQ ID NO:92 | VHH182511H8 | SEQ ID NO:84 |
| LIC182511H9 | SEQ ID NO:93 | VHH182511H9 | SEQ ID NO:85 |

The steps of transduction to generate CAR-T cells are shown in Example 4. 12 days after the cell transduction, cells were harvested by centrifugation at 300 g for 10 minutes at room temperature. Cell pellets were re-suspended with cell culture medium or DPBS and aliquoted and went through another centrifugation at 300 g for 10 minutes at room temperature. The cell pellets were then re-suspended using cell culture medium or DPBS for further use. Aliquots of DPBS re-suspended cells were processed for CAR expression levels assessment by flow cytometry.

### In Vitro Cytotoxicity Assay of CAR-T Cells

Cytotoxicity assay was performed after CAR-T cells being prepared and co-incubated with PANC1.huCLDN18.2.Luc, PANC1.huCLDN18.1.Luc, and NUGC4.Luc cells, respectively, at 8:1 or 2:1 effector (CAR-T cell) to target cell ratio (E:T) for 20-24 hours. Untransduced T cells serve as control. 175DX CAR-T cells serve as benchmark control. CD19 CAR-T cells, which target human CD19 antigen, serve as negative control.

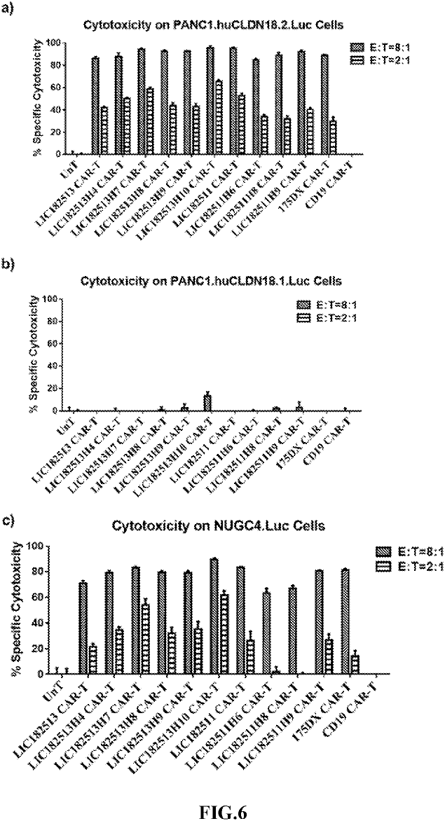

As shown in FIG. 6 (parts a-c), the humanized CAR-T cells showed potent killing effects to PANC1.huCLDN18.2.Luc cells which stably over-expressed human Claudin18.2 (see FIG. 6, part a) and gastric cancer cell line NUGC4.Luc which were also positive for human Claudin18.2 expression (see FIG. 6, part c). However, these humanized CAR-T did not show significant cytotoxicity on PANC1.huCLDN18.1.Luc cells which is negative for human Claudin18.2 expression but positive for human Claudin18.1 expression (see FIG. 6, part b). These result suggested that such humanized CAR-T cells were specific to human Claudin18.2 and not specific to human Claudin18.1. The benchmark control (175DX CAR-T cells) also showed such trend. The negative control CD19 CAR-T cells did not show cytotoxicity to either any of these 3 target cells which were CD19 negative.

As shown in FIG. 6 (parts a-c), especially at lower E:T ratio (E:T=2:1), VHH based CAR-T cells, including LIC182513, LIC182513H4, LIC182513H7, LIC182513H8, LIC182513H9, LIC182513H10, LIC182511, and LIC182511H9 CAR-T cells showed higher killing efficiencies on NUGC4.Luc (ranging from 21.45± 2.61% to 61.68±3.45% for VHH based CAR-T versus 14.00±4.64% for 175DX CAR-T ) and PANC1.huCLDN18.2.Luc cells (ranging from 40.44± 1.79% to 65.44±1.70% for VHH based CAR-T versus 30.10±3.78% for 175DX CAR-T ). Interestingly, humanized versions of LIC182513 CAR-T cells, including LIC182513H4, LIC182513H7, LIC182513H8, LIC182513H9, and LIC182513H10 CAR-T cells showed higher killing on-target efficiencies on NUGC4.Luc (ranging from 32.08± 4.45% to 61.68±3.45% for humanize VHH based CAR-T versus 21.45±2.61% for parental CAR-T) and PANC1.huCLDN18.2.Luc cells (ranging from 43.12± 2.37% to 65.44±1.70% for VHH based CAR-T versus 42.14± 1.05% for parental CAR-T ). While after humanization, LIC182511H9 CAR-T showed comparable killing efficiencies on NUGC4.Luc cells or less potency on PANC1.huCLDN18.2.Luc cells as to its parental clone LIC182511 CAR-T; however, the rest of the clones did not show potency improvement to its parental clone LIC182511 CAR-T.

### Evaluation of CAR-T Cells Cytokine Release

Supernatants from in vitro co-culture assays were collected for analysis of CAR specific cytokine release (interferon gamma or IFNγ) using HTRF kit (Cisbio, Cat#62HIFNGPEG). Briefly, HTRF reagents were allowed to warm up to room temperature for at least 30 minutes before the assay. 16 µL/well supernatants from co-culture assay were transferred to 384 well assay plate (Greiner Bio-One, #784075), followed by adding with 4 µL/well pre-mixed HTRF reagents prepared according to the kit manual. The plate were then sealed with parafilms and incubated overnight at room temperature. The next day, the plate were read on an HTRF compatible reader Tecan Spark 10 M. IFNγ concentration was calculated by referring to the signal obtained by standard curves provided by the kit.

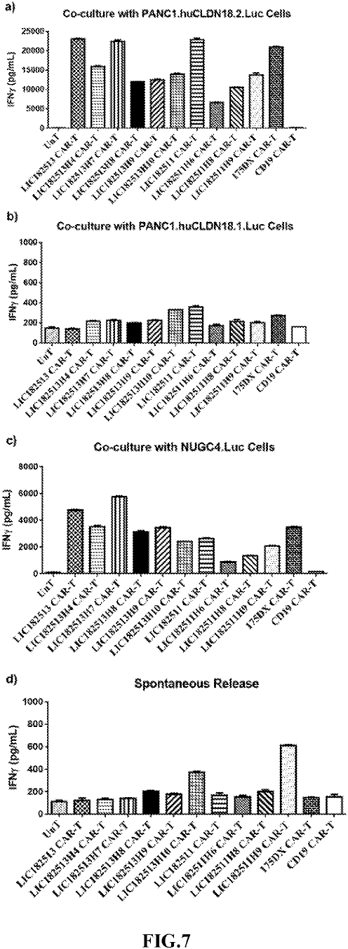

As shown in FIG. 7 (parts a and c), all of these Claudin18.2 CAR-T showed significant IFNy release upon co-culture with PANC1.huCLDN18.2.Luc cell (ranging from 6597.11±112.14 pg/mL to 23019.15±242.28 pg/mL for VHH based CAR-T versus 208.00±28.05 pg/mL for CD19 CAR-T) or NUGC4.Luc cells (ranging from 868.16±41.09 pg/mL to 5753.65±66.88 pg/mL for VHH based CAR-T versus 155.10±6.86 pg/mL for CD19 CAR-T). All of the tested CAR-T cells showed low levels of spontaneous IFNγ release (see FIG. 7, part d), except for LIC182513H10 and LIC182511H9 CAR-T cells which had slightly higher spontaneous IFNγ release than that by UnT or CD19 CAR-T. Such results indicated that, these VHH based CAR-T cells could release IFNy in human Claudin18.2 specific manner.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed:

1. A binding moiety that specifically binds to Claudin18.2, comprising a single domain antibody or an antigen binding fragment thereof comprising:
(i) a CDR1 comprising an amino acid sequence selected from the group consisting of SEQ ID NOs: 1-11 and 113-125;
(ii) a CDR2 comprising an amino acid sequence selected from the group consisting of SEQ ID NOs: 12-23; and
(iii) a CDR3 comprising an amino acid sequence selected from the group consisting of SEQ ID NOs:24-37 and 126-139.

2. The binding moiety according to claim 1, wherein the single domain antibody or antigen binding fragment thereof comprises:
(1) a CDR1 comprising the amino acid sequence of SEQ ID NO: 1 or SEQ ID NO: 113; a CDR2 comprising the amino acid sequence of SEQ ID NO: 12; and a CDR3 comprising the amino acid sequence of SEQ ID NO:24 or SEQ ID NO: 126;
(2) a CDR1 comprising the amino acid sequence of SEQ ID NO:2 or SEQ ID NO:114; a CDR2 comprising the amino acid sequence of SEQ ID NO: 13; and a CDR3 comprising the amino acid sequence of SEQ ID NO:25 or SEQ ID NO: 127;
(3) a CDR1 comprising the amino acid sequence of SEQ ID NO:3 or SEQ ID NO:115; a CDR2 comprising the amino acid sequence of SEQ ID NO: 14; and a CDR3 comprising the amino acid sequence of SEQ ID NO:26 or SEQ ID NO: 128;
(4) a CDR1 comprising the amino acid sequence of SEQ ID NO: 4 or SEQ ID NO:116; a CDR2 comprising the amino acid sequence of SEQ ID NO: 15; and a CDR3 comprising the amino acid sequence of SEQ ID NO:27 or SEQ ID NO: 129;
(5) a CDR1 comprising the amino acid sequence of SEQ ID NO:5 or SEQ ID NO:117; a CDR2 comprising the amino acid sequence of SEQ ID NO: 16; and a CDR3 comprising the amino acid sequence of SEQ ID NO:28 or SEQ ID NO: 130;
(6) a CDR1 comprising the amino acid sequence of SEQ ID NO:6 or SEQ ID NO:118; a CDR2 comprising the amino acid sequence of SEQ ID NO: 17; and a CDR3 comprising the amino acid sequence of SEQ ID NO:29 or SEQ ID NO: 131;
(7) a CDR1 comprising the amino acid sequence of SEQ ID NO:7 or SEQ ID NO:119; a CDR2 comprising the amino acid sequence of SEQ ID NO: 18; and a CDR3 comprising the amino acid sequence of SEQ ID NO:30 or SEQ ID NO: 132;
(8) a CDR1 comprising the amino acid sequence of SEQ ID NO:8 or SEQ ID NO: 120; a CDR2 comprising the amino acid sequence of SEQ ID NO: 19; and a CDR3 comprising the amino acid sequence of SEQ ID NO:31 or SEQ ID NO: 133;
(9) a CDR1 comprising the amino acid sequence of SEQ ID NO: 1 or SEQ ID NO: 121; a CDR2 comprising the amino acid sequence of SEQ ID NO:20; and a CDR3 comprising the amino acid sequence of SEQ ID NO:32 or SEQ ID NO: 134;
(10) a CDR1 comprising the amino acid sequence of SEQ ID NO:5 or SEQ ID NO: 122; a CDR2 comprising the amino acid sequence of SEQ ID NO:21; and a CDR3 comprising the amino acid sequence of SEQ ID NO:33 or SEQ ID NO: 135;
(11) a CDR1 comprising the amino acid sequence of SEQ ID NO:9 or SEQ ID NO: 123; a CDR2 comprising the amino acid sequence of SEQ ID NO:22; and a CDR3 comprising the amino acid sequence of SEQ ID NO:34 or SEQ ID NO: 136;
(12) a CDR1 comprising the amino acid sequence of SEQ ID NO:10 or SEQ ID NO: 124; a CDR2 comprising the amino acid sequence of SEQ ID NO:23; and a CDR3 comprising the amino acid sequence of SEQ ID NO:35 or SEQ ID NO: 137;
(13) a CDR1 comprising the amino acid sequence of SEQ ID NO:5 or SEQ ID NO:122; a CDR2 comprising the amino acid sequence of SEQ ID NO:21; and a CDR3 comprising the amino acid sequence of SEQ ID NO:36 or SEQ ID NO: 138; or
(14) a CDR1 comprising the amino acid sequence of SEQ ID NO: 11 or SEQ ID NO: 125; a CDR2 comprising the amino acid sequence of SEQ ID NO: 13 and a CDR3 comprising the amino acid sequence of SEQ ID NO:37 or SEQ ID NO: 139.

3. A binding moiety that specifically binds to Claudin18.2, comprising a single domain antibody or an antigen binding fragment thereof comprising:
(i) a CDR1, a CDR2, and a CDR3 having the amino acid sequences of the CDR1, CDR2, and CDR3, respectively, as set forth in SEQ ID NO:38;
(ii) a CDR1, a CDR2, and a CDR3 having the amino acid sequences of the CDR1, CDR2, and CDR3, respectively, as set forth in SEQ ID NO:39;
(iii) a CDR1, a CDR2, and a CDR3 having the amino acid sequences of the CDR1, CDR2, and CDR3, respectively, as set forth in SEQ ID NO:40;
(iv) a CDR1, a CDR2, and a CDR3 having the amino acid sequences of the CDR1, CDR2, and CDR3, respectively, as set forth in SEQ ID NO:41;
(v) a CDR1, a CDR2, and a CDR3 having the amino acid sequences of the CDR1, CDR2, and CDR3, respectively, as set forth in SEQ ID NO:42;
(vi) a CDR1, a CDR2, and a CDR3 having the amino acid sequences of the CDR1, CDR2, and CDR3, respectively, as set forth in SEQ ID NO:43;
(vii) a CDR1, a CDR2, and a CDR3 having the amino acid sequences of the CDR1, CDR2, and CDR3, respectively, as set forth in SEQ ID NO:44;
(viii) a CDR1, a CDR2, and a CDR3 having the amino acid sequences of the CDR1, CDR2, and CDR3, respectively, as set forth in SEQ ID NO:45;
(ix) a CDR1, a CDR2, and a CDR3 having the amino acid sequences of the CDR1, CDR2, and CDR3, respectively, as set forth in SEQ ID NO:46;
(x) a CDR1, a CDR2, and a CDR3 having the amino acid sequences of the CDR1, CDR2, and CDR3, respectively, as set forth in SEQ ID NO:47;
(xi) a CDR1, a CDR2, and a CDR3 having the amino acid sequences of the CDR1, CDR2, and CDR3, respectively, as set forth in SEQ ID NO:48;
(xii) a CDR1, a CDR2, and a CDR3 having the amino acid sequences of the CDR1, CDR2, and CDR3, respectively, as set forth in SEQ ID NO:49;
(xiii) a CDR1, a CDR2, and a CDR3 having the amino acid sequences of the CDR1, CDR2, and CDR3, respectively, as set forth in SEQ ID NO:50;
(xiv) a CDR1, a CDR2, and a CDR3 having the amino acid sequences of the CDR1, CDR2, and CDR3, respectively, as set forth in SEQ ID NO:51;
(xv) a CDR1, a CDR2, and a CDR3 having the amino acid sequences of the CDR1, CDR2, and CDR3, respectively, as set forth in SEQ ID NO:77;
(xvi) a CDR1, a CDR2, and a CDR3 having the amino acid sequences of the CDR1, CDR2, and CDR3, respectively, as set forth in SEQ ID NO:78;
(xvii) a CDR1, a CDR2, and a CDR3 having the amino acid sequences of the CDR1, CDR2, and CDR3, respectively, as set forth in SEQ ID NO:79;
(xviii) a CDR1, a CDR2, and a CDR3 having the amino acid sequences of the CDR1, CDR2, and CDR3, respectively, as set forth in SEQ ID NO:80;
(xix) a CDR1, a CDR2, and a CDR3 having the amino acid sequences of the CDR1, CDR2, and CDR3, respectively, as set forth in SEQ ID NO:81;
(xx) a CDR1, a CDR2, and a CDR3 having the amino acid sequences of the CDR1, CDR2, and CDR3, respectively, as set forth in SEQ ID NO:82;
(xxi) a CDR1, a CDR2, and a CDR3 having the amino acid sequences of the CDR1, CDR2, and CDR3, respectively, as set forth in SEQ ID NO:83;
(xxii) a CDR1, a CDR2, and a CDR3 having the amino acid sequences of the CDR1, CDR2, and CDR3, respectively, as set forth in SEQ ID NO:84 or
(xxiii) a CDR1, a CDR2, and a CDR3 having the amino acid sequences of the CDR1, CDR2, and CDR3, respectively, as set forth in SEQ ID NO:85.

4. The binding moiety of claim 3, wherein the CDR1, CDR2 or CDR3 are determined according to the Kabat numbering scheme, the IMGT numbering scheme, the AbM numbering scheme, the Chothia numbering scheme, the Contact numbering scheme, or a combination thereof.

5. The binding moiety of any one of claims 1 to 4, further comprising one or more FR regions as set forth in SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:77, SEQ ID NO:78, SEQ ID NO:79, SEQ ID NO:80, SEQ ID NO:81, SEQ ID NO:82, SEQ ID NO:83, SEQ ID NO:84, and/or SEQ ID NO:85.

6. The binding moiety according to any one of claims 1 to 5, wherein the single domain antibody or antigen binding fragment is camelid, chimeric, human or humanized.

7. The binding moiety according to any one of claims 1 to 6, wherein the single domain antibody or antigen binding fragment comprises a VHH domain.

8. The binding moiety according to any one of claims 1 to 7, wherein the single domain antibody or antigen binding fragment thereof comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to any one of SEQ ID NOs: 38-51 and 77-85.

9. The binding moiety according to any one of claims 1 to 8, wherein the single domain antibody or antigen binding fragment thereof comprises an IgG, IgM or IgA heavy chain constant region or a fragment thereof.

10. The binding moiety according to any one of claims 1 to 9, wherein the single domain antibody or antigen binding fragment thereof is genetically fused or chemically conjugated to an agent.

11. A polynucleotide encoding the binding moiety according to any one of claims 1 to 9.

12. A vector comprising the polynucleotide according to claim 11.

13. A host cell comprising the polynucleotide according to claim 11 or the vector according to claim 12.

14. A chimeric antigen receptor (CAR) comprising a polypeptide comprising:
(a) an extracellular antigen binding domain comprising the binding moiety according to any one of claims 1 to 10;
(b) a transmembrane domain; and
(c) an intracellular signaling domain.

15. The CAR of claim 14, wherein the extracellular antigen binding domain further comprises one or more additional antigen binding domain(s).

16. The CAR of claim 14, wherein the extracellular antigen binding domain further comprises one additional antigen binding domain; or wherein the extracellular antigen binding domain further comprises two additional antigen binding domains.

17. The CAR of any one of claims 14 to 16, wherein the transmembrane domain is derived from a molecule selected from a group consisting of CD8α, CD4, CD28, CD137, CD80, CD86, CD152, and PD1.

18. The CAR of claim 17, wherein the transmembrane domain is derived from CD8a.

19. The CAR of any one of claims 14 to 18, wherein the intracellular signaling domain comprises a primary intracellular signaling domain of an immune effector cell.

20. The CAR of claim 19, wherein the primary intracellular signaling domain is derived from CD3-zeta.

21. The CAR of claim 19 or claim 20, wherein the intracellular signaling domain further comprises a co-stimulatory signaling domain.

22. The CAR of claim 21, wherein the co-stimulatory signaling domain is derived from a co-stimulatory molecule selected from the group consisting of CD27, CD28, CD137, OX40, CD30, CD40, CD3, LFA-1, CD2, CD7, LIGHT, NKG2C, B7-H3, ligands of CD83 and combinations thereof.

23. The CAR of claim 22, wherein the co-stimulatory signaling domain is derived from CD137.

24. The CAR of any one of claims 14 to 23, further comprising a hinge domain located between the C-terminus of the extracellular antigen binding domain and the N-terminus of the transmembrane domain.

25. The CAR of claim 24, wherein the hinge domain is derived from CD8a.

26. The CAR of any one of claims 14 to 25, further comprising a signal peptide located at the N-terminus of the polypeptide.

27. The CAR of claim 26, wherein the signal peptide is derived from CD8a.

28. The CAR according to any one of claims 14 to 27, wherein the polypeptide comprises, from N-terminus to C-terminus, a signal peptide, an antigen binding domain comprising a VHH domain, a hinge domain, a transmembrane domain, a primary intracellular signaling domain and a co-stimulatory signaling domain.

29. The CAR according to claim 28, wherein the polypeptide comprises from N-terminus to C-terminus, a signal peptide derived from CD8α, an antigen binding domain comprising a VHH domain, a hinge domain derived from CD8α, a transmembrane domain derived from CD8α, a CD137 cytoplasmic domain , and a CD3-zeta’s cytoplasmic domain.

30. The CAR according to claim 29, wherein the polypeptide comprises from N-terminus to C-terminus, a signal peptide of SEQ ID NO:67, an antigen binding domain comprising a VHH domain, a hinge domain of SEQ ID NO:68, a transmembrane domain of SEQ ID NO:69, a CD137 cytoplasmic domain of SEQ ID NO:70, and a CD3-zeta’s cytoplasmic domain of SEQ ID NO:72.

31. The CAR according to claim 14, wherein the polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NOs:53-66 and 86-93.

32. A polynucleotide encoding the CAR according to any one of claims 14 to 31.

33. A vector comprising the polynucleotide according to claim 32.

34. A host cell comprising the polynucleotide according to claim 32 or the vector according to claim 33.

35. An engineered immune cell that recombinantly expresses the CAR according to any one of claims 14 to 31.

36. The engineered immune cell according to claim 35, wherein the engineered immune cell is a T cell.

37. A pharmaceutical composition comprising a therapeutically effective amount of the binding moiety according to any one of claims 1-10, the chimeric antigen receptor according to any one of claims 14 to 31, the polynucleotide according to claim 11 or 32, the vector according to claim 12 or 33, the host cell according to claim 13 or 34, or the engineered immune cell according to claim 35 or 36, and a pharmaceutically acceptable excipient.

38. A method for treating a Claudin18.2-expressing tumor or cancer in a subject in need thereof, comprising administering to the subject a therapeutically effective amount of the pharmaceutical composition of claim 37.

39. The method according to claim 38, wherein the Claudin18.2-expressing tumor or cancer is gastric, esophageal, gastroesophageal, pancreatic, ovarian, or lung tumor or cancer.


SEQUENCE LISTING (139 sequences)

SEQ ID NO: 1 - protein, 5 aa, Artificial Sequence
  CDR1
RYGMT

SEQ ID NO: 2 - protein, 5 aa, Artificial Sequence
  CDR1
AKCMA

SEQ ID NO: 3 - protein, 5 aa, Artificial Sequence
  CDR1
AYYMG

SEQ ID NO: 4 - protein, 5 aa, Artificial Sequence
  CDR1
LYCMG

SEQ ID NO: 5 - protein, 5 aa, Artificial Sequence
  CDR1
SVCMG

SEQ ID NO: 6 - protein, 5 aa, Artificial Sequence
  CDR1
SGDMN

SEQ ID NO: 7 - protein, 5 aa, Artificial Sequence
  CDR1
CNYMS

SEQ ID NO: 8 - protein, 5 aa, Artificial Sequence
  CDR1
SYCMS

SEQ ID NO: 9 - protein, 5 aa, Artificial Sequence
  CDR1
TYCMG

SEQ ID NO: 10 - protein, 5 aa, Artificial Sequence
  CDR1
TANMA

SEQ ID NO: 11 - protein, 5 aa, Artificial Sequence
  CDR1
SKCMA

SEQ ID NO: 12 - protein, 16 aa, Artificial Sequence
  CDR2
SISISGSTEYADSVKG

SEQ ID NO: 13 - protein, 17 aa, Artificial Sequence
  CDR2
TIQSGYYRPYYTDSVKG

SEQ ID NO: 14 - protein, 16 aa, Artificial Sequence
  CDR2
VIPGGTQTYYADSVKG

SEQ ID NO: 15 - protein, 16 aa, Artificial Sequence
  CDR2
AIDYYGSTTYADSVKG

SEQ ID NO: 16 - protein, 16 aa, Artificial Sequence
  CDR2
LIGRTGSTTYIDSVKG

SEQ ID NO: 17 - protein, 16 aa, Artificial Sequence
  CDR2
GINRAGNTNYAASVKG

SEQ ID NO: 18 - protein, 16 aa, Artificial Sequence
  CDR2
RIFPSGKTTYADSMKA

SEQ ID NO: 19 - protein, 17 aa, Artificial Sequence
  CDR2
AISTRGGSTYYVDSVKG

SEQ ID NO: 20 - protein, 16 aa, Artificial Sequence
  CDR2
SISLSGSTEYADSVKG

SEQ ID NO: 21 - protein, 16 aa, Artificial Sequence
  CDR2
VIGRDGSTTYIDSVKG

SEQ ID NO: 22 - protein, 17 aa, Artificial Sequence
  CDR2
NVYMRNGDTWLADSVKG

SEQ ID NO: 23 - protein, 16 aa, Artificial Sequence
  CDR2
AAHSDGSTKYADSVKG

SEQ ID NO: 24 - protein, 7 aa, Artificial Sequence
  CDR3
YNAYTQY

SEQ ID NO: 25 - protein, 20 aa, Artificial Sequence
  CDR3
DRFTYPPGVICGGISENYRY

SEQ ID NO: 26 - protein, 16 aa, Artificial Sequence
  CDR3
TYLVGSAPLSPSRFRY

SEQ ID NO: 27 - protein, 15 aa, Artificial Sequence
  CDR3
RRTSWCSVSFNGYDD

SEQ ID NO: 28 - protein, 11 aa, Artificial Sequence
  CDR3
GLGYWDCDYNY

SEQ ID NO: 29 - protein, 14 aa, Artificial Sequence
  CDR3
TYCRMALFGACNDY

SEQ ID NO: 30 - protein, 13 aa, Artificial Sequence
  CDR3
SCRGPGSIWPSDY

SEQ ID NO: 31 - protein, 18 aa, Artificial Sequence
  CDR3
AEGLCGWGGWESAVRFGS

SEQ ID NO: 32 - protein, 7 aa, Artificial Sequence
  CDR3
YNAYGQY

SEQ ID NO: 33 - protein, 11 aa, Artificial Sequence
  CDR3
GLGYWACEYDY

SEQ ID NO: 34 - protein, 12 aa, Artificial Sequence
  CDR3
GRSSCLEASISY

SEQ ID NO: 35 - protein, 19 aa, Artificial Sequence
  CDR3
RGSPWWNSVVALESYAYSY

SEQ ID NO: 36 - protein, 11 aa, Artificial Sequence
  CDR3
GLGYWACEYNY

SEQ ID NO: 37 - protein, 20 aa, Artificial Sequence
  CDR3
DRFTYPYGVICGGISANYRY

SEQ ID NO: 38 - protein, 115 aa, Artificial Sequence
  VHH182501 amino acid sequence
EVQLVESGGGSVQAGGSLRLSCAASGYTTSRYGMTWYRQAPAKEREFVSSISISGSTEYADSVKGRFTISHDNVKKTVYLQMNSLKPEDTAMYYCSFYNAYTQYWGQGTQVTVSS

SEQ ID NO: 39 - protein, 129 aa, Artificial Sequence
  VHH182502 amino acid sequence
EVQLVESGGGSVQPGGSLSLSCAASGDTFRAKCMAWFRQGPGKEREVVATIQSGYYRPYYTDSVKGRFTISQDADKNRVYLQMDSLKPEDTAMYYCAADRFTYPPGVICGGISENYRYWGQGTRVTVSS

SEQ ID NO: 40 - protein, 125 aa, Artificial Sequence
  VHH182503 amino acid sequence
EVQLVESGGGSVQAGGSLRLSCVASGTIYSAYYMGWFRQPPGKEHEGVATVIPGGTQTYYADSVKGRFTMSRDNAKNTVYLQMNSLKPEDTGMYYCAATYLVGSAPLSPSRFRYWGQGTQVTVSS

SEQ ID NO: 41 - protein, 123 aa, Artificial Sequence
  VHH182504 amino acid sequence
EVQLVESGGGSVQAGGSLRLSCEVTSYTVRLYCMGWFRQDSEKAREGVAAIDYYGSTTYADSVKGRFTISRDNAKNTLYLQMNSLKPEDSAVYYCASRRTSWCSVSFNGYDDWGQGTQVTVSS

SEQ ID NO: 42 - protein, 118 aa, Artificial Sequence
  VHH182505 amino acid sequence
QVQLVESGGGSVQVGGSLRLSCAASGYRASVCMGWFRQAPGKERERVALIGRTGSTTYIDSVKGRFTISRDSAKNTLSLQMDNLKPEDTAMYSCAAGLGYWDCDYNYWGQGTQVTVSS

SEQ ID NO: 43 - protein, 122 aa, Artificial Sequence
  VHH182506 amino acid sequence
QVHLVESGGGSVQAGGSLTLSCVVSGAEYYSGDMNWYRQAPGKEREFVSGINRAGNTNYAASVKGRFSISRDSARTTGYLQMNSLKSEDTAMYYCQGTYCRMALFGACNDYWGQGTQVTVSS

SEQ ID NO: 44 - protein, 120 aa, Artificial Sequence
  VHH182507 amino acid sequence
QVQLAESGGGSVQAGGSLKLICTASRATDCNYMSWHRQAPGKEREFVSRIFPSGKTTYADSMKARFSISQDNAKNSVYLLMVNLKPEDTGKYYCQGSCRGPGSIWPSDYWGQGTQVTVSS

SEQ ID NO: 45 - protein, 127 aa, Artificial Sequence
  VHH182508 amino acid sequence
EVQLAESGGGSVQAGGSLRLSCAASRYISSSYCMSWFRQAPGKEREGVAAISTRGGSTYYVDSVKGRFTIFQGNAGATVYLQMNSLKPEDTAMYYCAAAEGLCGWGGWESAVRFGSWGQGTQVTVSS

SEQ ID NO: 46 - protein, 115 aa, Artificial Sequence
  VHH182509 amino acid sequence
EVQLAESGGGSVQAGGSLRLSCAASGYITSRYGMTWYRQAPAKEREFVSSISLSGSTEYADSVKGRFTISHDNVKKTVYLQMNSLKPEDTAMYYCSFYNAYGQYWGQGIQVTVSS

SEQ ID NO: 47 - protein, 118 aa, Artificial Sequence
  VHH182510 amino acid sequence
QVRLVESGGGSVQVGGSLRLSCAASGYRSSVCMGWFRQAPGKERERVAVIGRDGSTTYIDSVKGRFTISRDSAKNTLSLQMDNLKPEDTAMYSCAAGLGYWACEYDYSGQGTQVTVSS

SEQ ID NO: 48 - protein, 118 aa, Artificial Sequence
  VHH182511 amino acid sequence
QVQLDESGGGSVQAGGSLRLSCAASRSTYCMGWFRQAPGKLRERVANVYMRNGDTWLADSVKGRFTISRDNAKNAVYLQMTRLRPEDTAMYYCAAGRSSCLEASISYWGQGTQVTVSS

SEQ ID NO: 49 - protein, 124 aa, Artificial Sequence
  VHH182512 amino acid sequence
EVQLVESGGGSVQAGGSLTLSCEASRSTANMAWFRQGPGKEREGVAAAHSDGSTKYADSVKGRFTISKDNGKNTLYLRMNGLKPEEGVMYYCAARGSPWWNSVVALESYAYSYWGQGTQVTVSS

SEQ ID NO: 50 - protein, 118 aa, Artificial Sequence
  VHH182513 amino acid sequence
QVQLEESGGGSVQVGGSLRLSCAASGYRSSVCMGWFRQAPGKERERVAVIGRDGSTTYIDSVKGRFTISRDSAKNTLSLQMDNLKPEDTAMYSCAAGLGYWACEYNYWGQGTQVTVSS

SEQ ID NO: 51 - protein, 129 aa, Artificial Sequence
  VHH182514 amino acid sequence
QVQLVESGGGSVQAGGSLSLSCAASGDTFHSKCMAWFRQGPGKEREVVATIQSGYYRPYYTDSVKGRFTISRDADKNRVYLQMDSLKPEDTAMYYCAADRFTYPYGVICGGISANYRYWGQGTRVTVSS

SEQ ID NO: 52 - protein, 249 aa, Artificial Sequence
  175DX amino acid sequence
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKGSTSGSGKPGSGEGSTKGQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 53 - protein, 361 aa, Artificial Sequence
  LIC182501 amino acid sequence
MALPVTALLLPLALLLHAARPEVQLVESGGGSVQAGGSLRLSCAASGYTTSRYGMTWYRQAPAKEREFVSSISISGSTEYADSVKGRFTISHDNVKKTVYLQMNSLKPEDTAMYYCSFYNAYTQYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 375 aa, Artificial Sequence
  LIC182502 amino acid sequence
MALPVTALLLPLALLLHAARPEVQLVESGGGSVQPGGSLSLSCAASGDTFRAKCMAWFRQGPGKEREVVATIQSGYYRPYYTDSVKGRFTISQDADKNRVYLQMDSLKPEDTAMYYCAADRFTYPPGVICGGISENYRYWGQGTRVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 371 aa, Artificial Sequence
  LIC182503 amino acid sequence
MALPVTALLLPLALLLHAARPEVQLVESGGGSVQAGGSLRLSCVASGTIYSAYYMGWFRQPPGKEHEGVATVIPGGTQTYYADSVKGRFTMSRDNAKNTVYLQMNSLKPEDTGMYYCAATYLVGSAPLSPSRFRYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 369 aa, Artificial Sequence
  LIC182504 amino acid sequence
MALPVTALLLPLALLLHAARPEVQLVESGGGSVQAGGSLRLSCEVTSYTVRLYCMGWFRQDSEKAREGVAAIDYYGSTTYADSVKGRFTISRDNAKNTLYLQMNSLKPEDSAVYYCASRRTSWCSVSFNGYDDWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 364 aa, Artificial Sequence
  LIC182505 amino acid sequence
MALPVTALLLPLALLLHAARPQVQLVESGGGSVQVGGSLRLSCAASGYRASVCMGWFRQAPGKERERVALIGRTGSTTYIDSVKGRFTISRDSAKNTLSLQMDNLKPEDTAMYSCAAGLGYWDCDYNYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 368 aa, Artificial Sequence
  LIC182506 amino acid sequence
MALPVTALLLPLALLLHAARPQVHLVESGGGSVQAGGSLTLSCVVSGAEYYSGDMNWYRQAPGKEREFVSGINRAGNTNYAASVKGRFSISRDSARTTGYLQMNSLKSEDTAMYYCQGTYCRMALFGACNDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 366 aa, Artificial Sequence
  LIC182507 amino acid sequence
MALPVTALLLPLALLLHAARPQVQLAESGGGSVQAGGSLKLICTASRATDCNYMSWHRQAPGKEREFVSRIFPSGKTTYADSMKARFSISQDNAKNSVYLLMVNLKPEDTGKYYCQGSCRGPGSIWPSDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 373 aa, Artificial Sequence
  LIC182508 amino acid sequence
MALPVTALLLPLALLLHAARPEVQLAESGGGSVQAGGSLRLSCAASRYISSSYCMSWFRQAPGKEREGVAAISTRGGSTYYVDSVKGRFTIFQGNAGATVYLQMNSLKPEDTAMYYCAAAEGLCGWGGWESAVRFGSWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 361 aa, Artificial Sequence
  LIC182509 amino acid sequence
MALPVTALLLPLALLLHAARPEVQLAESGGGSVQAGGSLRLSCAASGYITSRYGMTWYRQAPAKEREFVSSISLSGSTEYADSVKGRFTISHDNVKKTVYLQMNSLKPEDTAMYYCSFYNAYGQYWGQGIQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 364 aa, Artificial Sequence
  LIC182510 amino acid sequence
MALPVTALLLPLALLLHAARPQVRLVESGGGSVQVGGSLRLSCAASGYRSSVCMGWFRQAPGKERERVAVIGRDGSTTYIDSVKGRFTISRDSAKNTLSLQMDNLKPEDTAMYSCAAGLGYWACEYDYSGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 364 aa, Artificial Sequence
  LIC182511 amino acid sequence
MALPVTALLLPLALLLHAARPQVQLDESGGGSVQAGGSLRLSCAASRSTYCMGWFRQAPGKLRERVANVYMRNGDTWLADSVKGRFTISRDNAKNAVYLQMTRLRPEDTAMYYCAAGRSSCLEASISYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 370 aa, Artificial Sequence
  LIC182512 amino acid sequence
MALPVTALLLPLALLLHAARPEVQLVESGGGSVQAGGSLTLSCEASRSTANMAWFRQGPGKEREGVAAAHSDGSTKYADSVKGRFTISKDNGKNTLYLRMNGLKPEEGVMYYCAARGSPWWNSVVALESYAYSYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 364 aa, Artificial Sequence
  LIC182513 amino acid sequence
MALPVTALLLPLALLLHAARPQVQLEESGGGSVQVGGSLRLSCAASGYRSSVCMGWFRQAPGKERERVAVIGRDGSTTYIDSVKGRFTISRDSAKNTLSLQMDNLKPEDTAMYSCAAGLGYWACEYNYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 375 aa, Artificial Sequence
  LIC182514 amino acid sequence
MALPVTALLLPLALLLHAARPQVQLVESGGGSVQAGGSLSLSCAASGDTFHSKCMAWFRQGPGKEREVVATIQSGYYRPYYTDSVKGRFTISRDADKNRVYLQMDSLKPEDTAMYYCAADRFTYPYGVICGGISANYRYWGQGTRVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 21 aa, Artificial Sequence
  CD8alpha signal peptide
MALPVTALLLPLALLLHAARP

SEQ ID NO: 68 - protein, 45 aa, Artificial Sequence
  CD8alpha hinge
TTTPAPRPPTPAPTIASQPLSLRPEACRPAAGGAVHTRGLDFACD

SEQ ID NO: 69 - protein, 24 aa, Artificial Sequence
  CD8alpha transmembrane domain
IYIWAPLAGTCGVLLLSLVITLYC

SEQ ID NO: 70 - protein, 42 aa, Artificial Sequence
  4-1BB (CD137) cytoplasmic domain
KRGRKKLLYIFKQPFMRPVQTTQEEDGCSCRFPEEEEGGCEL

SEQ ID NO: 71 - protein, 41 aa, Artificial Sequence
  CD28 cytoplasmic domain
RSKRSRLLHSDYMNMTPRRPGPTRKHYQPYAPPRDFAAYRS

SEQ ID NO: 72 - protein, 112 aa, Artificial Sequence
  CD3zeta (CD3z) cytoplasmic domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 18 aa, Artificial Sequence
  Linker 1
GSTSGSGKPGSGEGSTKG

SEQ ID NO: 74 - protein, 2 aa, Artificial Sequence
  Linker 2
TS

SEQ ID NO: 75 - protein, 15 aa, Artificial Sequence
  Linker 3
GGGGSGGGGSGGGGS

SEQ ID NO: 76 - protein, 227 aa, Artificial Sequence
  Human IgG1Fc amino acid
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 118 aa, Artificial Sequence
  VHH182513H1 amino acid sequence
QVQLVESGGGLVQPGGSLRLSCAASGYRSSVCMGWFRQAPGKERERVAVIGRDGSTTYIDSVKGRFTISRDSAKNTLSLQMDSLRAEDTAMYSCAAGLGYWACEYNYWGQGTLVTVSS

SEQ ID NO: 78 - protein, 118 aa, Artificial Sequence
  VHH182513H4 amino acid sequence
QVQLVESGGGVVQPGRSLRLSCAASGYRSSVCMGWFRQAPGKGLERVAVIGRDGSTTYIDSVKGRFTISRDSAKNTLYLQMNSLRAEDTAMYYCAAGLGYWACEYNYWGQGTLVTVSS

SEQ ID NO: 79 - protein, 118 aa, Artificial Sequence
  VHH182513H7 amino acid sequence
QVQLEESGGGSVQVGGSLRLSCAASGYRSSVCMGWVRQAPGKGLEWVAVIGRDGSTTYIDSVKGRFTISRDSAKNTLSLQMDNLKPEDTAMYSCAAGLGYWACEYNYWGQGTQVTVSS

SEQ ID NO: 80 - protein, 118 aa, Artificial Sequence
  VHH182513H8 amino acid sequence
QVQLVESGGGVVQPGRSLRLSCAASGYRSSVCMGWFRQAPGKGLERVAVIGRDGSTTYIDSVKGRFTISRDNSKNTLYLQMNSLRAEDTAVYYCAAGLGYWACEYNYWGQGTLVTVSS

SEQ ID NO: 81 - protein, 118 aa, Artificial Sequence
  VHH182513H9 amino acid sequence
QVQLVESGGGVVQPGRSLRLSCAASGYRSSVCMGWFRQAPGKGLERVAVIGRDGSTTYIDSVKGRFTISRDSAKNTLYLQMNSLRAEDTAVYYCAAGLGYWACEYNYWGQGTLVTVSS

SEQ ID NO: 82 - protein, 118 aa, Artificial Sequence
  VHH182513H10 amino acid sequence
QVQLVESGGGVVQPGRSLRLSCAASGYRSSVCMGWFRQAPGKGLERVAVIGRDGSTTYIDSVKGRFTISRDNSKNTLYLQMNSLRAEDTAVYYCARGLGYWACEYNYWGQGTLVTVSS

SEQ ID NO: 83 - protein, 118 aa, Artificial Sequence
  VHH182511H6 amino acid sequence
QVQLDESGGGLVQPGGSLRLSCAASRSTYCMGWFRQAPGKGLERVANVYMRNGDTWLADSVKGRFTISRDNSKNTLYLQMTSLRAEDTAVYYCAAGRSSCLEASISYWGQGTLVTVSS

SEQ ID NO: 84 - protein, 118 aa, Artificial Sequence
  VHH182511H8 amino acid sequence
EVQLLESGGGLVQPGGSLRLSCAASRSTYCMGWFRQAPGKGLERVANVYMRNGDTWLADSVKGRFTISRDNSKNAVYLQMNSLRAEDTAVYYCAAGRSSCLEASISYWGQGTLVTVSS

SEQ ID NO: 85 - protein, 118 aa, Artificial Sequence
  VHH182511H9 amino acid sequence
QVQLLESGGGLVQPGGSLRLSCAASRSTYCMGWFRQAPGKGLERVANVYMRNGDTWLADSVKGRFTISRDNSKNAVYLQMTRLRAEDTAVYYCAAGRSSCLEASISYWGQGTLVTVSS

SEQ ID NO: 86 - protein, 364 aa, Artificial Sequence
  LIC182513H4 amino acid sequence
MALPVTALLLPLALLLHAARPQVQLVESGGGVVQPGRSLRLSCAASGYRSSVCMGWFRQAPGKGLERVAVIGRDGSTTYIDSVKGRFTISRDSAKNTLYLQMNSLRAEDTAMYYCAAGLGYWACEYNYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 364 aa, Artificial Sequence
  LIC182513H7 amino acid sequence
MALPVTALLLPLALLLHAARPQVQLEESGGGSVQVGGSLRLSCAASGYRSSVCMGWVRQAPGKGLEWVAVIGRDGSTTYIDSVKGRFTISRDSAKNTLSLQMDNLKPEDTAMYSCAAGLGYWACEYNYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 364 aa, Artificial Sequence
  LIC182513H8 amino acid sequence
MALPVTALLLPLALLLHAARPQVQLVESGGGVVQPGRSLRLSCAASGYRSSVCMGWFRQAPGKGLERVAVIGRDGSTTYIDSVKGRFTISRDNSKNTLYLQMNSLRAEDTAVYYCAAGLGYWACEYNYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 364 aa, Artificial Sequence
  LIC182513H9 amino acid sequence
MALPVTALLLPLALLLHAARPQVQLVESGGGVVQPGRSLRLSCAASGYRSSVCMGWFRQAPGKGLERVAVIGRDGSTTYIDSVKGRFTISRDSAKNTLYLQMNSLRAEDTAVYYCAAGLGYWACEYNYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 364 aa, Artificial Sequence
  LIC182513H10 amino acid sequence
MALPVTALLLPLALLLHAARPQVQLVESGGGVVQPGRSLRLSCAASGYRSSVCMGWFRQAPGKGLERVAVIGRDGSTTYIDSVKGRFTISRDNSKNTLYLQMNSLRAEDTAVYYCARGLGYWACEYNYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 364 aa, Artificial Sequence
  LIC182511H6 amino acid sequence
MALPVTALLLPLALLLHAARPQVQLDESGGGLVQPGGSLRLSCAASRSTYCMGWFRQAPGKGLERVANVYMRNGDTWLADSVKGRFTISRDNSKNTLYLQMTSLRAEDTAVYYCAAGRSSCLEASISYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 364 aa, Artificial Sequence
  LIC182511H8 amino acid sequence
MALPVTALLLPLALLLHAARPEVQLLESGGGLVQPGGSLRLSCAASRSTYCMGWFRQAPGKGLERVANVYMRNGDTWLADSVKGRFTISRDNSKNAVYLQMNSLRAEDTAVYYCAAGRSSCLEASISYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 364 aa, Artificial Sequence
  LIC182511H9 amino acid sequence
MALPVTALLLPLALLLHAARPQVQLLESGGGLVQPGGSLRLSCAASRSTYCMGWFRQAPGKGLERVANVYMRNGDTWLADSVKGRFTISRDNSKNAVYLQMTRLRAEDTAVYYCAAGRSSCLEASISYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 2 aa, Artificial Sequence
  Exemplary peptide linker 1
  REPEAT (1)..(2): 'GS' can be repeated at least 1, 2, 3, 4, 5, or 6 times
GS

SEQ ID NO: 95 - protein, 5 aa, Artificial Sequence
  Exemplary peptide linker 2
  REPEAT (1)..(5): 'GSGGS' can be repeated at least 1, 2, 3, 4, 5, or 6 times
GSGGS

SEQ ID NO: 96 - protein, 4 aa, Artificial Sequence
  Exemplary peptide linker 3
  REPEAT (1)..(4): 'GGGS' can be repeated at least 1, 2, 3, 4, 5, or 6 times
GGGS

SEQ ID NO: 97 - protein, 34 aa, Artificial Sequence
  Exemplary peptide linker 4
GGGGSGGGGSGGGGGGSGSGGGGSGGGGSGGGGS

SEQ ID NO: 98 - protein, 5 aa, Artificial Sequence
  Exemplary peptide linker 5
  REPEAT (1)..(5): 'GGGGS' can be repeated at least 1, 2, 3, 4, 5, or 6 times
GGGGS

SEQ ID NO: 99 - protein, 5 aa, Artificial Sequence
  Exemplary peptide linker 6
DGGGS

SEQ ID NO: 100 - protein, 5 aa, Artificial Sequence
  Exemplary peptide linker 7
TGEKP

SEQ ID NO: 101 - protein, 4 aa, Artificial Sequence
  Exemplary peptide linker 8
GGRR

SEQ ID NO: 102 - protein, 24 aa, Artificial Sequence
  Exemplary peptide linker 9
GGGGSGGGGSGGGGGGSGSGGGGS

SEQ ID NO: 103 - protein, 14 aa, Artificial Sequence
  Exemplary peptide linker 10
EGKSSGSGSESKVD

SEQ ID NO: 104 - protein, 16 aa, Artificial Sequence
  Exemplary peptide linker 11
KESGSVSSEQLAQFRS

SEQ ID NO: 105 - protein, 8 aa, Artificial Sequence
  Exemplary peptide linker 12
GGRRGGGS

SEQ ID NO: 106 - protein, 9 aa, Artificial Sequence
  Exemplary peptide linker 13
LRQRDGERP

SEQ ID NO: 107 - protein, 12 aa, Artificial Sequence
  Exemplary peptide linker 14
LRQKDGGGSERP

SEQ ID NO: 108 - protein, 16 aa, Artificial Sequence
  Exemplary peptide linker 15
LRQKDGGGSGGGSERP

SEQ ID NO: 109 - protein, 16 aa, Artificial Sequence
  Exemplary peptide linker 16
GSTSGSGKPGSGEGST

SEQ ID NO: 110 - protein, 14 aa, Artificial Sequence
  Exemplary peptide linker 17
GSTSGSGKSSEGKG

SEQ ID NO: 111 - protein, 18 aa, Artificial Sequence
  Exemplary peptide linker 18
KESGSVSSEQLAQFRSLD

SEQ ID NO: 112 - protein, 19 aa, Artificial Sequence
  Synthesized signal peptide
MGWSCIILFLVATATGVHS

SEQ ID NO: 113 - protein, 10 aa, Artificial Sequence
  CDR1
GYTTSRYGMT

SEQ ID NO: 114 - protein, 10 aa, Artificial Sequence
  CDR1
GDTFRAKCMA

SEQ ID NO: 115 - protein, 10 aa, Artificial Sequence
  CDR1
GTIYSAYYMG

SEQ ID NO: 116 - protein, 11 aa, Artificial Sequence
  CDR1
TSYTVRLYCMG

SEQ ID NO: 117 - protein, 9 aa, Artificial Sequence
  CDR1
GYRASVCMG

SEQ ID NO: 118 - protein, 10 aa, Artificial Sequence
  CDR1
GAEYYSGDMN

SEQ ID NO: 119 - protein, 9 aa, Artificial Sequence
  CDR1
RATDCNYMS

SEQ ID NO: 120 - protein, 10 aa, Artificial Sequence
  CDR1
RYISSSYCMS

SEQ ID NO: 121 - protein, 10 aa, Artificial Sequence
  CDR1
GYITSRYGMT

SEQ ID NO: 122 - protein, 9 aa, Artificial Sequence
  CDR1
GYRSSVCMG

SEQ ID NO: 123 - protein, 7 aa, Artificial Sequence
  CDR1
RSTYCMG

SEQ ID NO: 124 - protein, 7 aa, Artificial Sequence
  CDR1
RSTANMA

SEQ ID NO: 125 - protein, 10 aa, Artificial Sequence
  CDR1
GDTFHSKCMA

SEQ ID NO: 126 - protein, 8 aa, Artificial Sequence
  CDR3
FYNAYTQY

SEQ ID NO: 127 - protein, 21 aa, Artificial Sequence
  CDR3
ADRFTYPPGVICGGISENYRY

SEQ ID NO: 128 - protein, 17 aa, Artificial Sequence
  CDR3
ATYLVGSAPLSPSRFRY

SEQ ID NO: 129 - protein, 16 aa, Artificial Sequence
  CDR3
SRRTSWCSVSFNGYDD

SEQ ID NO: 130 - protein, 12 aa, Artificial Sequence
  CDR3
AGLGYWDCDYNY

SEQ ID NO: 131 - protein, 15 aa, Artificial Sequence
  CDR3
GTYCRMALFGACNDY

SEQ ID NO: 132 - protein, 14 aa, Artificial Sequence
  CDR3
GSCRGPGSIWPSDY

SEQ ID NO: 133 - protein, 19 aa, Artificial Sequence
  CDR3
AAEGLCGWGGWESAVRFGS

SEQ ID NO: 134 - protein, 8 aa, Artificial Sequence
  CDR3
FYNAYGQY

SEQ ID NO: 135 - protein, 12 aa, Artificial Sequence
  CDR3
AGLGYWACEYDY

SEQ ID NO: 136 - protein, 13 aa, Artificial Sequence
  CDR3
AGRSSCLEASISY

SEQ ID NO: 137 - protein, 20 aa, Artificial Sequence
  CDR3
ARGSPWWNSVVALESYAYSY

SEQ ID NO: 138 - protein, 12 aa, Artificial Sequence
  CDR3
AGLGYWACEYNY

SEQ ID NO: 139 - protein, 21 aa, Artificial Sequence
  CDR3
ADRFTYPYGVICGGISANYRY